(19) United States
(12) Patent Application Publication
(10) Pub. No.: US 2026/0250402 A1
(43) Pub. Date: Aug. 27, 2026

(54) IL-7Ra TARGETING ANTIBODIES AND USES THEREOF

(71) Applicant: c/o INMAGENE PTE. LTD.

(72) Inventors: Chongtian GUO, Shanghai (CN); Pengcheng FAN, Zhejiang (CN); Run LEI, Shanghai (CN); Xiaotong WU, Shanghai (CN); Steven XU, Zhejiang (CN)

(21) Appl. No.: 19/668,283

(22) Filed: May 5, 2026

Related U.S. Application Data
(63) Continuation of application No. PCT/CN2024/130002, filed on Nov. 5, 2024.

(30) Foreign Application Priority Data
Nov. 10, 2023 (WO) PCT/CN2023/131067

(51) Int. Cl. C07K 16/28

(52) U.S. Cl. CPC C07K 16/2866; C07K 2317/24

(57) ABSTRACT

Disclosed herein are anti-IL-7Ra antibodies and antigen-binding fragments, polynucleotides encoding the antibodies and antigen-binding fragments, and pharmaceutical compositions comprising the antibodies and antigen-binding fragments. Medical uses of the anti-IL-7Ra antibodies and antigen-binding fragments described herein are also disclosed.

### 1. CROSS-REFERENCE TO RELATED APPLICATION

This application claims the priority to PCT application No. PCT/CN2023/131067 filed on Nov. 10, 2023. The entire content of the aforementioned application is hereby incorporated by reference.

### 2. REFERENCE TO SEQUENCE LISTING SUBMITTED ELECTRONICALLY

This application incorporates by reference a Sequence Listing as an XML file entitled “[P24408196C].SEQ” created on Nov. 5, 2024 and having a size of 52,763 bytes.

### 3. FIELD

The present invention relates to molecular biology, cell biology, and immunology. Provided herein include anti-IL-7Ra antibodies and uses thereof in treating IL-7 and/or TSLP-associated immunological disorders.

### 4. BACKGROUND

Ulcerative colitis (UC) is a chronic and debilitating inflammatory bowel disease that affects millions of individuals worldwide. It is characterized by inflammation and ulcers in the lining of the colon and rectum, leading to a range of distressing symptoms, including abdominal pain, diarrhea, and rectal bleeding. While current treatments for UC have made significant strides in managing the disease and improving patients' quality of life, there remains a pressing need for more effective therapies.

Interleukin-7 receptor alpha (IL-7Ra) is a protein that plays a crucial role in regulating the immune system, which is involved in the overactive immune response responsible for the chronic inflammation and tissue damage in subjects with UC. Despite the therapeutic potential, IL-7Ra-targeting agents for treating UC are currently lacking. The compositions and methods provided herein address this need and provide related advantages.

### 5. SUMMARY

Provided herein are antibodies or antigen-binding fragments thereof that specifically bind human IL-7Ra, comprising: (1) as defined by Kabat, (a) a light chain variable region (VL) comprising VL CDR1, VL CDR2, VL CDR3 having the amino acid sequences of SEQ ID NOs: 8, 9, and 10, respectively; or a variant thereof having up to about 5 amino acid substitutions, additions, and/or deletions in the VL CDRs; and/or (b) a heavy chain variable region (VH) comprising VH CDR1, VH CDR2, VH CDR3 having the amino acid sequences of SEQ ID NOs: 12, 14 and 15, respectively; or a variant thereof having up to about 5 amino acid substitutions, additions, and/or deletions in the VH CDRs; or (2) as defined by Chothia, (a) a VL comprising VL CDR1, VL CDR2, VL CDR3 having the amino acid sequences of SEQ ID NOs: 8, 9, and 10, respectively; or a variant thereof having up to about 5 amino acid substitutions, additions, and/or deletions in the VL CDRs; and/or (b) a VH comprising VH CDR1, VH CDR2, VH CDR3 having the amino acid sequences of SEQ ID NOs: 11, 13, and 15, respectively; or a variant thereof having up to about 5 amino acid substitutions, additions, and/or deletions in the VH CDRs.

In some embodiments, the antibodies or antigen-binding fragments provided herein comprise VL CDR1, VL CDR2, VL CDR3, VH CDR1, VH CDR2 and VH CDR3 having the amino acid sequences of SEQ ID NOs: 8, 9, 10, 12, 14 and 15, respectively, as defined by Kabat.

In some embodiments, the antibodies or antigen-binding fragments provided herein comprise VL CDR1, VL CDR2, VL CDR3, VH CDR1, VH CDR2 and VH CDR3 having the amino acid sequences of SEQ ID NOs: 8, 9, 10, 11, 13, and 15, respectively, as defined by Chothia.

In some embodiments, provided herein are antibodies or antigen-binding fragments thereof that specifically bind human IL-7Ra, comprising: (a) a VL having at least 85%, at least 90%, at least 95%, at least 98%, or 100% sequence identity to the amino acid sequence of SEQ ID NO:18; and/or (b) a VH having at least 85%, at least 90%, at least 95%, at least 98%, or 100% sequence identity to the amino acid sequence of SEQ ID NO:19.

In some embodiments, the antibodies or antigen-binding fragments provided herein comprise a VL and a VH having the amino acid sequences of SEQ ID NOs: 18 and 19, respectively.

In some embodiments, provided herein are antibodies or antigen-binding fragments thereof that specifically bind human IL-7Ra, comprising (a) a VL comprising VL CDR1, VL CDR2, and VL CDR3 from a VL having the amino acid sequence of SEQ ID NO:18; and/or (b) a VH comprising VH CDR1, VH CDR2, and VH CDR3 from a VH having the amino acid sequence of SEQ ID NO: 19.

In some embodiments, the antibodies or antigen-binding fragments provided herein are chimeric antibodies or antigen-binding fragments, humanized antibodies or antigen-binding fragments, or human antibodies or antigen-binding fragments. In some embodiments, the antibodies or antigen-binding fragments provided herein are humanized antibodies or antigen-binding fragments.

In some embodiments, the antibodies or antigen-binding fragments provided herein comprise (a) a VL having at least 85%, at least 90%, at least 95%, at least 98%, or 100% sequence identity to an amino acid sequence selected from the group consisting of SEQ ID NOs: 20-23; and/or (b) a VH having at least 85%, at least 90%, at least 95%, at least 98%, or 100% sequence identity to an amino acid sequence selected from the group consisting of SEQ ID NOs: 26-30.

In some embodiments, the antibodies or antigen-binding fragments provided herein comprise a VL and a VH having the amino acid sequences of (1) SEQ ID NOs: 20 and 26, respectively; (2) SEQ ID NOs: 20 and 27, respectively; (3) SEQ ID NOs: 20 and 28, respectively; (4) SEQ ID NOs: 20 and 29, respectively; (5) SEQ ID NOs: 20 and 30, respectively; (6) SEQ ID NOs: 21 and 26, respectively; (7) SEQ ID NOs: 21 and 27, respectively; (8) SEQ ID NOs: 21 and 28, respectively; (9) SEQ ID NOs: 21 and 29, respectively; (10) SEQ ID NOs: 21 and 30, respectively; (11) SEQ ID NOs: 22 and 26, respectively; (12) SEQ ID NOs: 22 and 27, respectively; (13) SEQ ID NOs: 22 and 28, respectively; (14) SEQ ID NOs: 22 and 29, respectively; (15) SEQ ID NOs: 22 and 30, respectively; (16) SEQ ID NOs: 23 and 26, respectively; (17) SEQ ID NOs: 23 and 27, respectively; (18) SEQ ID NOs: 23 and 28, respectively; (19) SEQ ID NOs: 23 and 29, respectively; or (20) SEQ ID NOs: 23 and 30, respectively.

In some embodiments, the antibodies or antigen-binding fragments provided herein are selected from the group consisting of a Fab, a Fab′, a F(ab′)₂, a Fv, a scFv, a (scFv)₂, a single domain antibody (sdAb), and a heavy chain antibody (HCAb).

In some embodiments, the antibodies or antigen-binding fragments provided herein are IgG1 antibodies, IgG2 antibodies, IgG3 antibodies, or IgG4 antibodies.

In some embodiments, the antibodies provided herein comprise a light chain constant region (CL) having at least 85% sequence identity to kappa CL (CK; SEQ ID NO:33). In some embodiments, the antibodies provided herein comprise a light chain constant region (CL) having at least 85% sequence identity to lambda CL (CA; SEQ ID NO:34).

In some embodiments, the antibodies provided herein comprise a heavy chain constant region (CH) having at least 85% sequence identity to an amino acid sequence selected from the group consisting of SEQ ID NOs: 35-38.

In some embodiments, the antibodies provided herein are IgG4 antibodies.

In some embodiments, the heavy chain constant regions (CH) of the IgG4 antibodies provided herein comprise a wildtype IgG4 CH, or comprise at least one amino acid mutation. In some embodiments, the CH regions of the IgG4 antibodies provided herein have S228P substitution (SEQ ID NO:39).

In some embodiments, the Fc regions of the antibodies provided herein are afucosylated.

In some embodiments, provided herein are antibodies or antigen-binding fragments thereof that compete with the antibodies or antigen-binding fragments disclosed herein that specifically bind human IL-7Ra for binding to human IL-7Ra.

In some embodiments, the antibodies or antigen-binding fragments provided herein are bispecific antibodies or multispecific antibodies.

In some embodiments, the antibodies or antigen-binding fragments provided herein are monoclonal antibodies or antigen-binding fragments.

In some embodiments, the antibodies or antigen-binding fragments provided herein: (1) bind to human IL-7Ra with high affinity; (2) block IL-7 binding to IL-7Ra; (3) block IL-7-induced STAT5 phosphorylation; (4) block IL-7-induced T cell proliferation; (5) blocks TSLP induced TARC secretion; or (6) ameliorate colitis; or any combination of (1)-(6).

In some embodiments, provided herein are antibodies or antigen-binding fragments thereof that specifically bind human IL-7Ra, wherein the antibodies or antigen-binding fragments: (1) bind to human IL-7Ra with high affinity; (2) block IL-7 binding to IL-7Ra; (3) block IL-7-induced STAT5 phosphorylation; (4) block IL-7-induced T cell proliferation; (5) blocks TSLP induced TARC secretion; or (6) ameliorate colitis; or any combination of (1)-(6).

In some embodiments, provided herein are polynucleotides encoding a polypeptide of the antibody or antigen-binding fragment disclosed herein.

In some embodiments, provided herein are vectors comprising a polynucleotide disclosed herein.

In some embodiments, provided herein are host cells comprising a polynucleotide disclosed herein, or a vector disclosed herein.

In some embodiments, the host cells disclosed herein (1) overexpress N-acetylglucosaminyltransferase III (GnTIII), (2) lack α-1,6-fucosyltransferase (FUT8), or (3) have a low fucose content, or any combination of (1)-(3).

In some embodiments, provided herein are methods of making an antibody or antigen-binding fragment thereof that specifically binds human IL-7Ra, comprising culturing a cell disclosed herein under conditions that allow expression of the antibody or antigen-binding fragment. In some embodiments, the methods provided herein comprise isolating the antibody or antigen-binding fragment from the culture.

In some embodiments, provided herein are pharmaceutical compositions comprising a therapeutically effective amount of the antibody or antigen-binding fragment disclosed herein, and a pharmaceutically acceptable carrier.

In some embodiments, provided herein are methods of reducing IL-7 signaling and/or TSLP signaling in a subject in need thereof, comprising administering to the subject a therapeutically effective amount of the antibody or antigen-binding fragment disclosed herein.

In some embodiments, provided herein are methods of reducing autoimmunity or inflammation in a subject in need thereof, comprising administering to the subject an effective amount of the antibody or antigen-binding fragment disclosed herein.

In some embodiments, the subject has an autoimmune or inflammatory disease.

In some embodiments, provided herein are methods of treating an autoimmune or inflammatory disease associated with IL-7 and/or TSLP in a subject in need thereof, comprising administering to the subject a therapeutically effective amount of the antibody or antigen-binding fragment disclosed herein.

In some embodiments, the autoimmune or inflammatory disease is ulcerative colitis (UC).

In some embodiments, the methods provided herein further comprise administering an additional therapy to the subject.

In some embodiments, the subject is a human.

In some embodiments, provided herein are uses of the antibody or antigen-binding fragment disclosed herein in reducing IL-7 signaling and/or TSLP signaling.

In some embodiments, provided herein are uses of the antibody or antigen-binding fragment disclosed herein for the preparation of a medicament for reducing IL-7 signaling and/or TSLP signaling.

In some embodiments, provided herein are uses of the antibody or antigen-binding fragment disclosed herein in reducing autoimmunity or inflammation. In some embodiments, provided herein are uses of the antibody or antigen-binding fragment disclosed herein for the preparation of a medicament for reducing autoimmunity or inflammation.

In some embodiments, provided herein are uses of the antibody or antigen-binding fragment disclosed herein in treating an autoimmune or inflammatory disease associated with IL-7 and/or TSLP. In some embodiments, provided herein are uses of the antibody or antigen-binding fragment disclosed herein for the preparation of a medicament for treating an autoimmune or inflammatory disease associated with IL-7 and/or TSLP.

In some embodiments, the autoimmune or inflammatory disease is UC.

### 6. BRIEF DESCRIPTION OF DRAWINGS

FIG. 1 provides representative ELISA results showing the binding of the candidate chimeric anti-IL-7Ra antibodies (cmAb004, cmAb006, cmAb011 and cmAb018), reference antibodies (Tab1 and Tab2) and negative control antibodies (hIgG1 and hIgG4) to human (left) and cynomolgus (right) IL-7Ra protein.

FIGS. 2A-2B provide representative flow cytometry results showing the binding of the candidate chimeric anti-IL-7Ra antibodies (cmAb004, cmAb006, cmAb007, cmAb009, cmAb011 and cmAb018), reference antibodies (Tab1 and Tab2) and negative control antibodies (hIgG1 and hIgG4) to CHOK1-human IL-7Ra cells, CHOK1-cyno IL-7Ra cells and CHOK1-blank cells. Results for cmAb018 are provided in FIG. 2B, and results for the rest of the antibodies are provided in FIG. 2A.

FIG. 3 provides representative flow cytometry results showing the inhibitory activities of candidate chimeric anti-IL-7Ra antibodies (cmAb004, cmAb006, cmAb007, cmAb009, cmAb011 and cmAb018), reference antibodies (Tab1 and Tab2) and negative control antibodies (hIgG1 and hIgG4) on IL-7 binding to CHOK1-human IL-7Ra cells.

FIG. 4 provides representative flow cytometry results showing the inhibitory activities of candidate chimeric anti-IL-7Ra antibodies (cmAb004, cmAb006, cmAb007, cmAb009, cmAb011 and cmAb018), reference antibodies (Tab1 and Tab2) and negative control antibodies (hIgG1 and hIgG4) on IL-7-induced STAT5 phosphorylation in human PBMCs.

FIGS. 5A-5D provide representative flow cytometry results showing the inhibitory activities of candidate chimeric anti-IL-7Ra antibodies (cmAb004, cmAb006, cmAb007, cmAb009, cmAb011 and cmAb018), reference antibodies (Tab1 and Tab2) and negative control antibodies (hIgG1 and hIgG4) on IL-7-induced T cell proliferation in five donors' PBMCs. FIG. 5A depicts results from donor 1 cells; FIG. 5B depicts results from donor 2 cells; FIG. 5C depicts results from donor 3 cells; and FIG. 5D depicts results from donor 4 and donor 5 cells.

FIG. 6 provides representative flow cytometry results showing the binding of anti-IL-7Ra chimeric antibody (cmAb011), anti-IL-7Ra humanized antibodies (HuAb001-020), reference antibodies (Tab1 and Tab2) and negative control antibody (Isotype) to CHOK1-human IL-7Ra cells, CHOK1-cyno IL-7Ra cells and CHOK1-blank cells.

FIG. 7 provides representative flow cytometry results showing the inhibitory activities of anti-IL-7Ra chimeric antibody (cmAb011), selected anti-IL-7Ra humanized antibodies (HuAb001- 009, 011, 013, 015), reference antibodies (Tab1 and Tab2) and negative control antibody (Isotype) on IL-7 binding to CHOK1-human IL-7Ra cells.

FIG. 8 provides representative flow cytometry results showing the inhibitory activities of anti-IL-7Ra chimeric antibody (cmAb011), selected anti-IL-7Ra humanized antibodies (HuAb003-005, 008-009, 013), reference antibodies (Tab1 and Tab2) and negative control antibody (Isotype) on IL-7-induced STAT5 phosphorylation in human PBMCs.

FIGS. 9A-9B provide representative flow cytometry results showing the inhibitory activities of anti-IL-7Ra chimeric antibody (cmAb011) compared with selected anti-IL-7Ra humanized antibodies (HuAb003-005, 008-009, 013) on IL-7-induced T cell proliferation in PBMCs (FIG. 9A), and the inhibitory activities of anti-IL-7Ra humanized antibody HuAb on IL-7-induced T cell proliferation compared with reference antibodies (Tab1 and Tab2) and negative control antibodies (hIgG1 and hIgG4) in three donors' PBMCs (FIG. 9B).

FIG. 10 provides representative results showing body weight tracking of humanized M-NSG mice modeled with TNBS and hIL-7 to induce colitis, and protective effect of anti-IL-7Ra humanized antibody HuAb on body weight loss compared with reference antibody (Tab2) and negative control antibody (Isotype).

FIG. 11 provides representative ELISA results showing the inhibitory activities of anti-IL-7Ra humanized antibody HuAb on TSLP induced TARC secretion compared with reference antibodies (Tab1 and Tab2) and negative control antibodies (hIgG1 and hIgG4) in PBMCs.

### 7. DETAILED DESCRIPTION

The present disclosure provides novel antibodies, including antigen-binding fragments that specifically bind IL-7Ra (e.g., human IL-7Ra). Pharmaceutical compositions comprising a therapeutically effective amount of such antibodies or antigen-binding fragments are also disclosed herein. Also disclosed herein are uses of such pharmaceutical compositions for treating autoimmune or inflammatory diseases associated with IL-7 and/or TSLP.

IL-7Ra, also known as CD127, is a shared receptor by the IL-7 receptor (IL-7R) and the thymic stromal lymphopoietin (TSLP) receptor (TSLPR) for heterodimerization. The IL-7 receptor is a heterodimer comprising IL-7Ra and the common gamma chain (γc) of interleukin receptors. The TSLP receptor is a heterodimer of IL-7Ra and cytokine receptor-like factor 2 (CRLF2).

Human IL-7Ra exist in four isoforms (Swiss Prot P16871). The canonical isoform has a total of 459 amino acids (SEQ ID NO:1), including a 20 amino acid signal sequence, a 219 amino acid extracellular region, a 25 amino acid transmembrane region and a 195 amino acid intracellular region.

(SEQ ID NO: 1)
MTILGTTFGMVFSLLQVVSGESGYAQNGDLEDAELDDYSFSCYSQLEVN
GSQHSLTCAFEDPDVNITNLEFEICGALVEVKCLNFRKLQEIYFIETKK
FLLIGKSNICVKVGEKSLTCKKIDLTTIVKPEAPFDLSVVYREGANDFV
VTFNTSHLQKKYVKVLMHDVAYRQEKDENKWTHVNLSSTKLTLLQRKLQ
PAAMYEIKVRSIPDHYFKGFWSEWSPSYYFRTPEINNSSGEMDPILLTI
SILSFFSVALLVILACVLWKKRIKPIVWPSLPDHKKTLEHLCKKPRKNL
NVSFNPESFLDCQIHRVDDIQARDEVEGFLQDTFPQQLEESEKQRLGGD
VQSPNCPSEDVVITPESFGRDSSLTCLAGNVSACDAPILSSSRSLDCRE
SGKNGPHVYQDLLLSLGTTNSTLPPPFSLQSGILTLNPVAQGQPILTSL
GSNQEEAYVTMSSFYQNQ

A Cytokine Receptor Homology class I (CRH I) receptor, the extracellular domain of IL-7Ra consists of two fibronectin 3 domains, termed D1 and D2. The precise crystallographic structure of IL-7Ra is disclosed in the Research Collaboratory for Structural Bioinformatics Protein Data Bank (RCSB PDB) database (accession number 3UP1). D1 is involved in the binding with IL-7, while D2 is involved in the binding to the γc chain (and also with IL-7).

IL-7Ra is mainly expressed on T (mainly Naïve T, memory T) cells, NK cells, B cell progenitors, innate lymphoid cells (ILCs), and lymphatic endothelia cells. IL-7Ra is critical for development and maintenance of the entire lymphoid compartment. IL-7/IL-7Ra/γc is needed for T cell survival, TCR independent proliferation, memory T cell lasting. TSLP/TSLPR/IL-7Ra is responsible for barrier response. More information about human IL-7Ra can be found on public databases with the following IDs: HGNC: 6024; NCBI Entrez Gene: 3575; Ensembl: ENSG00000168685; OMIM®: 146661; UniProtKB/Swiss-Prot: P16871.

The sequence of cynomolgus IL-7Ra is provided below (UniProt No: Q38IC7):

(SEQ ID NO: 2)
MTILGTTFGMVFSLLQVVSGESGYAQNGDLEDAELDDYSFSCYSQLEVN
GSQHSLTCAFEDPDVNTTNLEFEICGALVEVKCLSFRKLQEIYFIETKK
FLLIGKSNICVKVGGKSLTCKKIDLTTIVKPEAPFDLSVIYREGANDFV
VTFNTSHLQKKYVKVLMHDVAYRQEKDENKWMHVNLSSTKLTLLQRNLQ
PEAMYEIKVRSIPDHYFKGFWSEWSPSYYFRTPEINNSPGEMDPILLTI
SLLSFFSVALLVILACVLWKKRIKPIVWPSLPDHKKTLEHLCKKPRKNL
NVSFNPESFLDCQIHRVDDIQARDEVEGFLQDTFPQQLEESKKQRLGGD
VQSPSCPSEDVVITPESFERDSSLRCLAGNVSACDAPILSSSRSLDCRE
SGKNGPHVYQDLLLSLGTTNSTLPPPFSLQSGILTLNPVAQGQPILTSL
GSNQEEAYVTMSSFYQNQ

IL-7 receptor signaling. Binding of IL-7 to IL-7 receptor triggers the activation of several signaling pathways, including the Janus kinases (JAK)-1 and -3, signal transducer and activator of transcription 5 (STAT5) and phosphatidylinositol 3-kinase (PI3K). The activation of the STAT5 pathway is required for the induction of the anti-apoptotic protein Bcl-2 and the prevention of the entry of the pro-apoptotic protein Bax in the mitochondrion and thus for survival of thymic developing T cell precursors. The activation of the PI3K pathway results in the phosphorylation and cytoplasmic retention of the pro-apoptotic protein Bad.

TSLP receptor signaling. TSLP is an epithelial cell cytokine that is active in lymphopoiesis and in particular is involved in regulation of development of cells of the immune system, impacting the maturation of the cells. Human TSLP also exerts polarization of dendritic cells, promotes T and B cell proliferation and differentiation, and suppresses the generation of Treg cells.

IL-7Ra is among the top listed risk genes of many autoimmune and inflammatory diseases. IL-7 and TSLP ligands are also both highly expressed in a number of autoimmune and inflammatory diseases. Indeed, IL-7 and TSLP signaling plays complementary role behind common pathophysiology of immunological diseases. IL-7 signaling decreases threshold for autoimmune TCR activation locally and shifts balance between pathologic T cells and Tregs. TSLP signaling initiates cascade response to environmental challenge locally and promotes Th2, Th17 inflammation, fibrosis and itch sensation. Both IL-7 and TSLP signaling are induced by and responsible for steroid resistance. The anti-IL-7Ra antibodies disclosed herein can inhibit both pathways and are therefore useful in treating autoimmune or inflammatory diseases associated with IL-7 and/or TSLP, such as UC.

Before the present disclosure is further described, it is to be understood that the disclosure is not limited to the particular embodiments set forth herein, and it is also to be understood that the terminology used herein is for the purpose of describing particular embodiments, and is not intended to be limiting.

### 7.1 Definitions

Unless otherwise defined herein, scientific and technical terms used in the present disclosures shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The term “a” or “an” entity refers to one or more of that entity; for example, “an antibody,” is understood to represent one or more antibodies.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “about” is used to indicate that a value includes the inherent variation of error for the device, the method being employed to determine the value, or the variation that exists among the study subjects. The term “about” encompasses the exact number recited. In some embodiments, “about” means within plus or minus 10% of a given value or range. In certain embodiments, “about” means that the variation is ±5%, ±4%, ±3%, ±2%, ±1%, ±0.5%, ±0.2%, or ±0.1% of the value to which “about” refers. In some embodiments, “about” means that the variation is ±1%, ±0.5%, ±0.2%, or ±0.1% of the value to which “about” refers.

The term “antibody,” and its grammatical equivalents as used herein refer to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, single-domain antibodies (sdAbs; e.g., camelid antibodies, alpaca antibodies), single-chain Fv (scFv) antibodies, heavy chain antibodies (HCAbs), light chain antibodies (LCAbs), multispecific antibodies, bispecific antibodies, monospecific antibodies, monovalent antibodies, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, camel antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgAQ1 and IgA2), based on the identity of their heavy-chain constant domains referred to as alpha, delta, epsilon, gamma, and mu, respectively. Unless expressly indicated otherwise, the term “antibody” as used herein include “antigen-binding fragment” of intact antibodies. The term “antigen-binding fragment” as used herein refers to a portion or fragment of an intact antibody that is the antigenic determining variable region of an intact antibody. Examples of antigen-binding fragments include, but are not limited to, Fab, Fab′, F(ab′) 2, Fv, linear antibodies, single chain antibody molecules (e.g., scFv), heavy chain antibodies (HCAbs), light chain antibodies (LCAbs), disulfide-linked scFv (dsscFv), diabodies, tribodies, tetrabodies, minibodies, dual variable domain antibodies (DVD), single variable domain antibodies (sdAbs; e.g., camelid antibodies, alpaca antibodies), and single variable domain of heavy chain antibodies (VHH), and bispecific or multispecific antibodies formed from antibody fragments. A “bispecific” antibody is an artificial hybrid antibody having two different antigen binding sites, which recognize and specifically bind two different targets. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulin. In some instances, the Fv framework region residues of a human immunoglobulin are replaced with the corresponding residues in an antibody from a non-human species. In some instances, residues of the CDRs are replaced by residues from the CDRs of a non-human species (e.g., mouse, rat, hamster, camel) that have the desired specificity, affinity, and/or binding capability. The humanized antibody can be further modified by the substitution of additional residues either in the Fv framework region and/or within the replaced non-human residues to refine and optimize antibody specificity, affinity, and/or binding capability. The term “human antibody” as used herein refers to an antibody produced by a human or an antibody having an amino acid sequence corresponding to an antibody produced by a human made using any of the techniques known in the art.

The term “heavy chain” when used in reference to an antibody refers to a polypeptide chain of about 50-70 kDa, wherein the amino-terminal portion includes a variable region of about 120 to 130 or more amino acids and a carboxy-terminal portion that includes a constant region. The constant region can be one of five distinct types, referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while μ and ε contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The approximate length of a light chain is 211 to 217 amino acids. There are two distinct types, referred to as kappa (κ) and lambda (λ) based on the amino acid sequence of the constant domains. Light chain amino acid sequences are well known in the art. A light chain can be a human light chain.

The term “variable domain” or “variable region” refers to a portion of the light or heavy chains of an antibody that is generally located at the amino-terminal of the light or heavy chains and has a length of about 120 to 130 amino acids in the heavy chain and about 100 to 110 amino acids in the light chain, and is used in the binding and specificity of each particular antibody for its particular antigen. The variable domain of the light chain is referred to as the “VL”; and the variable domain of the heavy chain is referred to as the “VH.” The variable domains differ extensively in sequence between different antibodies. The variability in sequence is concentrated in the CDRs while the less variable portions in the variable domain are referred to as framework regions (FR). The CDRs of the light and heavy chains are primarily responsible for the interaction of the antibody with antigen. The numbering of amino acid positions used herein is according to the EU Index, as in Kabat et al. (1991) Sequences of proteins of immunological interest. (U.S. Department of Health and Human Services, Washington, D.C.) 5^{th} ed. A variable region can be a human variable region.

A CDR refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences. CDR regions are well known to those skilled in the art and have been defined by a variety of methods/systems. These systems and/or definitions have been developed and refined over years and include Kabat, Chothia, IMGT, AbM, and Contact. For example, Kabat defines the regions of most hypervariability within the antibody variable (V) domains (Kabat et al., J. Biol. Chem. 252:6609-6616 (1977); Kabat, Adv. Prot. Chem. 32:1-75 (1978)). The Chothia definition is based on the location of the structural loop regions, which defines CDR region sequences as those residues that are not part of the conserved β-sheet framework, and thus are able to adapt different conformations (Chothia and Lesk, J. Mol. Biol. 196:901-917 (1987)). Both terminologies are well recognized in the art. Additionally, the IMGT system is based on sequence variability and location within the structure of the variable regions. The AbM definition is a compromise between Kabat and Chothia. The Contact definition is based on analyses of the available antibody crystal structures. Software programs (e.g., abYsis) are available and known to those of skill in the art for analysis of antibody sequence and determination of CDRs. The positions of CDRs within a canonical antibody variable domain have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

For example, CDRs defined according to either the Kabat (hypervariable) or Chothia (structural) designations, are set forth in the table below.

| | Kabat¹ | Chothia² | Loop Location |
|---|---|---|---|
| VHCDRl | 31-35 | 26-32 | linking B and C strands |
| VHCDR2 | 50-65 | 53-55 | linking C′ and C″ strands |
| VHCDR3 | 95-102 | 96-101 | linking F and G strands |
| VLCDRl | 24-34 | 26-32 | linking B and C strands |
| VLCDR2 | 50-56 | 50-52 | linking C′ and C″ strands |
| VLCDR3 | 89-97 | 91-96 | linking F and G strands |

¹Residue numbering follows the nomenclature of Kabat et al., supra
²Residue numbering follows the nomenclature of Chothia et al., supra

One or more CDRs also can be incorporated into a molecule either covalently or noncovalently to make it an immunoadhesin. An immunoadhesin can incorporate the CDR(s) as part of a larger polypeptide chain, can covalently link the CDR(s) to another polypeptide chain, or can incorporate the CDR(s) noncovalently. The CDRs permit the immunoadhesin to bind to a particular antigen of interest. The CDR regions can be analyzed by, for example, abysis website (http://abysis.org/).

The terms “epitope” and “antigenic determinant” as used interchangeably herein refer to the site on the surface of a target molecule to which an antibody or antigen-binding fragment binds, such as a localized region on the surface of an antigen. The target molecule can comprise, a protein, a peptide, a nucleic acid, a carbohydrate, or a lipid. An epitope having immunogenic activity is a portion of a target molecule that elicits an immune response in an animal. An epitope of a target molecule having antigenic activity is a portion of the target molecule to which an antibody binds, as determined by any method well known in the art, including, for example, by an immunoassay. Antigenic epitopes need not necessarily be immunogenic. Epitopes often consist of chemically active surface groupings of molecules such as amino acids or sugar side chains and have specific three dimensional structural characteristics as well as specific charge characteristics. The term, “epitope” includes linear epitopes and conformational epitopes. A region of a target molecule (e.g., a polypeptide) contributing to an epitope can be contiguous amino acids of the polypeptide or the epitope can come together from two or more non-contiguous regions of the target molecule. The epitope may or may not be a three-dimensional surface feature of the target molecule. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

The term “specifically binds,” as used herein, means that a polypeptide or molecule interacts more frequently, more rapidly, with greater duration, with greater affinity, or with some combination of the above to the epitope, protein, or target molecule than with alternative substances, including related and unrelated proteins. A binding moiety (e.g., antibody) that specifically binds a target molecule (e.g., antigen) can be identified, for example, by immunoassays, ELISAs, Bio-Layer Interferometry (“BLI”), SPR (e.g., Biacore), or other techniques known to those of skill in the art. Typically, a specific reaction will be at least twice background signal or noise and can be more than 10 times background. See, e.g., Paul, ed., 1989, Fundamental Immunology Second Edition, Raven Press, New York at pages 332-336 for a discussion regarding antibody specificity. A binding moiety that specifically binds a target molecule can bind the target molecule at a higher affinity than its affinity for a different molecule. In some embodiments, a binding moiety that specifically binds a target molecule can bind the target molecule with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different molecule. In some embodiments, a binding moiety that specifically binds a particular target molecule binds a different molecule at such a low affinity that binding cannot be detected using an assay described herein or otherwise known in the art. In some embodiments, “specifically binds” means, for instance, that a binding moiety binds a molecule target with a K_{D} of about 0.1 mM or less. In some embodiments, “specifically binds” means that a polypeptide or molecule binds a target with a K_{D} of about 10 μM or less or about 1 μM or less. In some embodiments, “specifically binds” means that a polypeptide or molecule binds a target with a K_{D} of about 0.1 μM or less, about 0.01 μM or less, or about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a binding moiety (e.g., antibody) that specifically binds a first target may or may not specifically bind a second target. As such, “specific binding” does not necessarily require (although it can include) exclusive binding, i.e., binding to a single target. Thus, a binding moiety (e.g., antibody) can, in some embodiments, specifically bind more than one target. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities.

The term “binding affinity” as used herein generally refers to the strength of the sum total of noncovalent interactions between a binding moiety (e.g., antibody) and a target molecule (e.g., antigen). The binding of a binding moiety and a target molecule is a reversible process, and the affinity of the binding is typically reported as an equilibrium dissociation constant (K_{D}). K_{D} is the ratio of a dissociation rate (k_{off} or k_{d}) to the association rate (kₒₙ or kₐ). The lower the K_{D} of a binding pair, the higher the affinity. A variety of methods of measuring binding affinity are known in the art, any of which can be used for purposes of the present disclosure. Specific illustrative embodiments include the following. In some embodiments, the “K_{D}” or “K_{D} value” can be measured by assays known in the art, for example by a binding assay. The K_{D} may be measured in a radiolabeled antigen binding assay (RIA) (Chen, et al., (1999) J. Mol. Biol. 293:865-881). The K_{D} or K_{D} value can also be measured by using biolayer interferometry (BLI) using, for example, the Gator system (Probe Life), or the Octet-96 system (Sartorius AG). The K_{D} or K_{D} value can also be measured by using surface plasmon resonance assays (SPR) by Biacore, using, for example, a BIAcore™-2000 or a BIAcore™-3000 BIAcore, Inc., Piscataway, NJ). The binding affinity can also be quantified with EC₅₀, which is the concentration of ligand at which half of the target is present in the bound state in a binding assay.

The terms “polypeptide,” “peptide,” “protein,” and their grammatical equivalents as used interchangeably herein refer to polymers of amino acids of any length, which can be linear or branched. It can include unnatural or modified amino acids or be interrupted by non-amino acids. A polypeptide, peptide, or protein can also be modified with, for example, disulfide bond formation, glycosylation, lipidation, acetylation, phosphorylation, or any other manipulation or modification.

The term “variant” as used herein in relation to a protein or a polypeptide with particular sequence features (the “reference protein” or “reference polypeptide”) refers to a different protein or polypeptide having one or more (such as, for example, about 1 to about 30, about 1 to about 25, about 1 to about 20, about 1 to about 15, about 1 to about 10, or about 1 to about 5) amino acid substitutions, deletions, and/or additions as compared to the reference protein or reference polypeptide. The changes to an amino acid sequence can be amino acid substitutions. The changes to an amino acid sequence can be conservative amino acid substitutions. The changes to an amino acid sequence can be amino acid deletions. A variant can be a fragment of the reference protein or polypeptide. A functional variant of a protein or polypeptide maintains the basic structural and functional properties of the reference protein or polypeptide.

The terms “polynucleotide,” “nucleic acid,” and their grammatical equivalents as used interchangeably herein refer to a polymer or oligomer of nucleotides of any length. The nucleotides can be deoxyribonucleotides, ribonucleotides, modified nucleotides or bases (such as methylated, hydroxymethylated, or glycosylated), non-natural nucleotides, non-nucleotide building blocks that exhibit similar structure and/or function as natural nucleotides (i.e., “nucleotide analogs”), and/or any substrate that can be incorporated into a polymer by DNA or RNA polymerase. The nucleic acids or polynucleotides can be heterogenous or homogenous in composition, can be isolated from naturally occurring sources, or can be artificially or synthetically produced. In addition, the nucleic acids may be DNA or RNA, or a mixture thereof, and can exist permanently or transitionally in single-stranded or double-stranded form, including homoduplex, heteroduplex, and hybrid states. Nucleic acid structures also include, for instance, a DNA/RNA helix, peptide nucleic acid (PNA), morpholino nucleic acid (see, e.g., Braasch and Corey, Biochemistry, 4(14): 4503-4510 (2002) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acid (see Wang, Am. Chem. Soc., 122:8595-8602 (2000)), and/or a ribozyme.

The terms “identical,” percent “identity,” and their grammatical equivalents as used herein in the context of two or more polynucleotides or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two polynucleotides or polypeptides provided herein are substantially identical, meaning they have at least 70%, at least 75%, at least 80%, at least 85%, at least 90%, and in some embodiments at least 95%, at least 96%, at least 97%, at least 98%, or at least 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-100 bases or more, and in some embodiments the sequences are substantially identical over the full length of the sequences being compared, such as a nucleotide sequence encoding a protein of interest.

The term “vector,” and its grammatical equivalents as used herein refer to a vehicle that is used to carry genetic material (e.g., a polynucleotide sequence), which can be introduced into a host cell, where it can be replicated and/or expressed. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more polynucleotides are to be co-expressed, both polynucleotides can be inserted, for example, into a single expression vector or in separate expression vectors. For single vector expression, the encoding polynucleotides can be operationally linked to one common expression control sequence or linked to different expression control sequences, such as one inducible promoter and one constitutive promoter. The introduction of polynucleotides into a host cell can be confirmed using methods well known in the art. It is understood by those skilled in the art that the polynucleotides are expressed in a sufficient amount to produce a desired product (e.g., an anti-IL-7Ra antibody or antigen-binding fragment as described herein), and it is further understood that expression levels can be optimized to obtain sufficient expression using methods well known in the art.

As used herein, the term “encode” and its grammatical equivalents refer to the inherent property of specific sequences of nucleotides in a polynucleotide or a nucleic acid, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein. Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

A polypeptide, peptide, protein, antibody, polynucleotide, vector, cell, or composition which is “isolated” is a polypeptide, peptide, protein, antibody, polynucleotide, vector, cell, or composition which is in a form not found in nature. Isolated polypeptides, peptides, proteins, antibodies, polynucleotides, vectors, cells, or compositions include those which have been purified to a degree that they are no longer in a form in which they are found in nature. In some embodiments, a polypeptide, peptide, protein, antibody, polynucleotide, vector, cell, or composition which is isolated is substantially pure.

The term “treat” and its grammatical equivalents as used herein in connection with a disease or a condition, or a subject having a disease or a condition refer to an action that suppresses, eliminates, reduces, and/or ameliorates a symptom, the severity of the symptom, and/or the frequency of the symptom associated with the disease or disorder being treated.

The term “administer” and its grammatical equivalents as used herein refer to the act of delivering, or causing to be delivered, a therapeutic or a pharmaceutical composition to the body of a subject by a method described herein or otherwise known in the art. The therapeutic can be a compound, a polypeptide, an antibody, a cell, or a population of cells. Administering a therapeutic or a pharmaceutical composition includes prescribing a therapeutic or a pharmaceutical composition to be delivered into the body of a subject. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

The terms “effective amount,” “therapeutically effective amount,” and their grammatical equivalents as used herein refer to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to the subject. The therapeutically effective amount can be ascertained by measuring relevant physiological effects. The exact amount required varies from subject to subject, depending on the age, weight, and general condition of the subject, the severity of the condition being treated, the judgment of the clinician, and the like. An appropriate “effective amount” in any individual case can be determined by one of ordinary skill in the art using routine experimentation.

The term “pharmaceutically acceptable carrier” or “pharmaceutically acceptable excipient” refers to a material that is suitable for drug administration to an individual along with an active agent without causing undesirable biological effects or interacting in a deleterious manner with any of the other components of the pharmaceutical composition.

The term “subject” as used herein refers to any animal (e.g., a mammal), including, but not limited to, humans, non-human primates, canines, felines, rodents, and the like, which is to be the recipient of a particular treatment. A subject can be a human. A subject can have a particular disease or condition.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Exemplary genes and polypeptides are described herein with reference to GenBank numbers, GI numbers and/or SEQ ID NOs. It is understood that one skilled in the art can readily identify homologous sequences by reference to sequence sources, including but not limited to GenBank (ncbi.nlm.nih.gov/genbank/) and EMBL (embl.org/).

### 7.2 Anti-IL-7Ra Antibodies and Antigen-Binding Fragments

Provided herein are antibodies or antigen-binding fragments thereof that specifically bind IL-7Ra (e.g., human IL-7Ra). In some embodiments, provided herein are anti-IL-7Ra antibodies. In some embodiments, the antibody is an IgA, IgD, IgE, IgG, or IgM antibody. In some embodiments, the antibody is an IgA antibody. In some embodiments, the antibody is an IgD antibody. In some embodiments, the antibody is an IgE antibody. In some embodiments, the antibody is an IgG antibody. In some embodiments, the antibody is an IgM antibody. In some embodiments, the antibodies provided herein can be an IgG1 antibody, an IgG2 antibody, an IgG3 antibody, or an IgG4 antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, provided herein are antigen-binding fragments of an anti-IL-7Ra antibody. In some embodiments, antigen-binding fragments provided herein can be a single domain antibody (sdAb), a heavy chain antibody (HCAb), a Fab, a Fab′, a F(ab′)₂, a Fv, a single-chain variable fragment (scFv), or a (scFv)₂. In some embodiments, the antigen-binding fragment of an anti-IL-7Ra antibody is a single domain antibody (sdAb). In some embodiments, the antigen-binding fragment of an anti-IL-7Ra antibody is a heavy chain antibody (HCAb). In some embodiments, the antigen-binding fragment of an anti-IL-7Ra antibody is a Fab. In some embodiments, the antigen-binding fragment of an anti-IL-7Ra antibody is a Fab′. In some embodiments, the antigen-binding fragment of an anti-IL-7Ra antibody is a F(ab′)₂. In some embodiments, the antigen-binding fragment of an anti-IL-7Ra antibody is a Fv. In some embodiments, the antigen-binding fragment of an anti-IL-7Ra antibody is a scFv. In some embodiments, the antigen-binding fragment of an anti-IL-7Ra antibody is a disulfide-linked scFv [(scFv)₂]. In some embodiments, the antigen-binding fragment of an anti-IL-7Ra antibody is a diabody (dAb).

In some embodiments, the anti-IL-7Ra antibodies or antigen-binding fragments provided herein comprise recombinant antibodies or antigen-binding fragments. In some embodiments, the anti-IL-7Ra antibodies or antigen-binding fragments provided herein comprise monoclonal antibodies or antigen-binding fragments. In some embodiments, the anti-IL-7Ra antibodies or antigen-binding fragments provided herein comprise polyclonal antibodies or antigen-binding fragments. In some embodiments, the anti-IL-7Ra antibodies or antigen-binding fragments provided herein comprise camelid (e.g., camels, dromedary and llamas) antibodies or antigen-binding fragments. In some embodiments, the anti-IL-7Ra antibodies or antigen-binding fragments provided herein comprise chimeric antibodies or antigen-binding fragments. In some embodiments, the anti-IL-7Ra antibodies or antigen-binding fragments provided herein comprise humanized antibodies or antigen-binding fragments. In some embodiments, the anti-IL-7Ra antibodies or antigen-binding fragments provided herein comprise human antibodies or antigen-binding fragments. In some embodiments, provided herein are anti-IL-7Ra human scFvs.

In some embodiments, the anti-IL-7Ra antibodies or antigen-binding fragments provided herein are isolated. In some embodiments, the anti-IL-7Ra antibodies or antigen-binding fragments provided herein are substantially pure.

In some embodiments, the anti-IL-7Ra antibody or antigen-binding fragment provided herein comprises a multispecific antibody or antigen-binding fragment. In some embodiments, the anti-IL-7Ra antibody or antigen-binding fragment provided herein comprises a bispecific antibody or antigen-binding fragment. In some embodiments, the bispecific antibody or antigen-binding fragment comprises an anti-IL-7Ra antibody or antigen-binding fragment provided herein. In some embodiments, the bispecific antibody or antigen-binding fragment comprises an anti-IL-7Ra scFv provided herein.

In some embodiments, the anti-IL-7Ra antibody or antigen-binding fragment provided herein comprises a monovalent antigen-binding site. In some embodiments, an anti-IL-7Ra antibody or antigen-binding fragment comprises a monospecific binding site. In some embodiments, an anti-IL-7Ra antibody or antigen-binding fragment comprises a bivalent binding site.

In some embodiments, an anti-IL-7Ra antibody or antigen-binding fragment is a monoclonal antibody or antigen-binding fragment. Monoclonal antibodies can be prepared by any method known to those of skill in the art.

In some embodiments, a monoclonal antibody is modified by using recombinant DNA technology to generate alternative antibodies. In some embodiments, the constant domains of the light chain and heavy chain of a mouse monoclonal antibody are replaced with the constant regions of a human antibody to generate a chimeric antibody. In some embodiments, the constant regions are truncated or removed to generate a desired antibody fragment of a monoclonal antibody. In some embodiments, site-directed or high-density mutagenesis of the variable region(s) is used to optimize specificity and/or affinity of a monoclonal antibody.

In some embodiments, provided herein is the anti-IL-7Ra antibody clone Ab011. The sequence features are described below. The specific CDR sequences defined herein are generally based on either Kabat or Chothia definition. However, it is understood that a general reference to a heavy chain CDR or CDRs and/or a light chain CDR or CDRs of a specific antibody encompass all CDR definitions as known to those of skill in the art. In some embodiments, provided herein are anti-IL-7Ra antibodies having the VL CDRs and/or VH CDRs of antibody clone Ab011 disclosed herein, wherein the CDRs are defined by Kabat, Chothia, IMGT, AbM, or Contact. In some embodiments, the CDRs are defined by Kabat (as exemplified in detail below). In some embodiments, the CDRs are defined by Chothia (as exemplified in detail below). In some embodiments, the CDRs are defined by IMGT. In some embodiments, the CDRs are defined by AbM. In some embodiments, the CDRs are defined by Contact.

In some embodiments, the anti-IL-7Ra antibody or antigen-binding fragment thereof provided herein is the antibody designated as cmAb011 (chimeric Ab011). In some embodiments, the anti-IL-7Ra antibody or antigen-binding fragment thereof provided herein has a VL from cmAb011 (SEQ ID NO:18). In some embodiments, the anti-IL-7Ra antibody or antigen-binding fragment thereof provided herein has a VH from cmAb011 (SEQ ID NO:19). The anti-IL-7Ra antibody or antigen-binding fragment thereof provided herein can have both the VL and the VH from cmAb011. In some embodiments, the anti-IL-7Ra antibody or antigen-binding fragment thereof provided herein has a VL that comprises VL CDRs 1, 2, and 3 from the VL from cmAb011 (SEQ ID NO:18). In some embodiments, the anti-IL-7Ra antibody or antigen-binding fragment thereof provided herein has a VH that comprises VH CDRs 1, 2, and 3 from the VH from cmAb011 (SEQ ID NO:19). The anti-IL-7Ra antibody or antigen-binding fragment thereof provided herein can have a VL comprising VL CDRs 1, 2, and 3 and a VH comprising VH CDRs 1, 2, and 3 from the VL and VH of cmAb011, respectively. The CDRs can be defined by any system known in the art. In some embodiments, the CDRs are defined by Kabat, Chothia, IMGT, AbM, or Contact. In some embodiments, the CDRs are defined by Kabat or Chothia as detailed herein. In some embodiments, the CDRs are defined by IMGT. In some embodiments, the CDRs are defined by AbM. In some embodiments, the CDRs are defined by Contact.

In some embodiments, the anti-IL-7Ra antibody or antigen-binding fragment thereof provided herein is a variant of cmAb011. The cmAb011 variant can have a VL that is a variant of the VL of cmAb011 having up to about 3, about 5, about 8, about 10, about 12, or about 15 amino acid substitutions, additions, and/or deletions in SEQ ID NO:18. The cmAb011 variant can have a VL that is a variant of the VL of cmAb011 having up to about 5 amino acid substitutions, additions, and/or deletions in SEQ ID NO:18. The cmAb011 variant can have a VH that is a variant of the VH of cmAb011 having up to about 3, about 5, about 8, about 10, about 12, or about 15 amino acid substitutions, additions, and/or deletions in SEQ ID NO:19. The cmAb011 variant can have a VH that is a variant of the VH of cmAb011 having up to about 5 amino acid substitutions, additions, and/or deletions in SEQ ID NO: 19. The amino acid substitutions, additions, and/or deletions can be in the VH CDRs or VL CDRs. In some embodiments, the amino acid substitutions, additions, and/or deletions are not in the CDRs. In some embodiments, the variant of cmAb011 has up to about 5 conservative amino acid substitutions. In some embodiments, the variant of cmAb011 has up to 3 conservative amino acid substitutions. In some embodiments, the anti-IL-7Ra antibody or antigen-binding fragment thereof provided herein is a humanized antibody or antigen-binding fragment derived from cmAb011. In some embodiments, the anti-IL-7Ra antibody or antigen-binding fragment thereof provided herein is a human antibody or antigen-binding fragment derived from cmAb011.

In some embodiments, anti-IL-7Ra antibodies or antigen-binding fragments provided herein comprise one, two, three, four, five, and/or six CDRs of any one of the antibodies described herein. In some embodiments, anti-IL-7Ra antibodies or antigen-binding fragments provided herein comprise a light chain variable region (VL) comprising one, two, and/or three, light chain CDRs (VL CDRs) from Table 1. In some embodiments, anti-IL-7Ra antibodies or antigen-binding fragments provided herein comprise a heavy chain variable region (VH) comprising one, two, and/or three heavy chain CDRs (VH CDRs) from Table 2. In some embodiments, anti-IL-7Ra antibodies or antigen-binding fragments provided herein comprise one, two, and/or three VL CDRs from Table 1 and one, two, and/or three VH CDRs from Table 2.

TABLE 1
Amino acid sequences of light chain variable region CDRs (VL CDRs) of Ab011

| Sequence | VL CDR1 | VL CDR2 | VL CDR3 |
|---|---|---|---|
| Kabat | RTSESVSIHLLHLMH | GASNLES | QQSIKDPYT |
| | (SEQ ID NO: 8) | (SEQ ID NO: 9) | (SEQ ID NO: 10) |
| Chothia | RTSESVSIHLLHLMH | GASNLES | QQSIKDPYT |
| | (SEQ ID NO: 8) | (SEQ ID NO: 9) | (SEQ ID NO: 10) |

TABLE 2
Amino acid sequences of heavy chain variable region CDRs (VH CDRs) of Ab011

| Sequence | VH CDR1 | VH CDR2 | VH CDR3 |
|---|---|---|---|
| Kabat | DYELH | GIDPKTGGTAYNQKFKG | SGGRWYFDV |
| | (SEQ ID NO: 12) | (SEQ ID NO: 14) | (SEQ ID NO: 15) |
| Chothia | GYRFTDY | DPKTGG | SGGRWYFDV |
| | (SEQ ID NO: 11) | (SEQ ID NO: 13) | (SEQ ID NO: 15) |

In some embodiments, provided herein are antibodies or antigen-binding fragments thereof that specifically bind IL-7Ra (e.g., human IL-7Ra), comprising a VL comprising (1) a VL CDR1 having the amino acid sequence of SEQ ID NO:8; (2) a VL CDR2 having the amino acid sequence of SEQ ID NO:9; and/or (3) a VL CDR3 having the amino acid sequence of SEQ ID NO:10; or a variant thereof having up to about 3, about 5, about 8, about 10, about 12, or about 15 amino acid substitutions, additions, and/or deletions in the VL CDRs; and/or a VH comprising (1) a VH CDR1 having an amino acid sequence of SEQ ID NO: 11 or 12; (2) a VH CDR2 having an amino acid sequence of SEQ ID NO:13 or 14; and/or (3) a VH CDR3 having an amino acid sequence of SEQ ID NO: 15; or a variant thereof having up to about 3, about 5, about 8, about 10, about 12, or about 15 amino acid substitutions, additions, and/or deletions in the VH CDRs.

In some embodiments, provided herein are antibodies or antigen-binding fragments thereof that specifically bind IL-7Ra, comprising a VL comprising (1) a VL CDR1 having the amino acid sequence of SEQ ID NO:8; (2) a VL CDR2 having the amino acid sequence of SEQ ID NO:9; or (3) a VL CDR3 having the amino acid sequence of SEQ ID NO:10; or a variant thereof having up to about 3, about 5, about 8, about 10, about 12, or about 15 amino acid substitutions, additions, and/or deletions in the VL CDR. In some embodiments, the variant has up to about 5 amino acid substitutions, additions, and/or deletions in the VL CDR. In some embodiments, provided herein are antibodies or antigen-binding fragments thereof that specifically bind IL-7Ra, comprising a VL comprising (1) a VL CDR1 having the amino acid sequence of SEQ ID NO:8; (2) a VL CDR2 having the amino acid sequence of SEQ ID NO:9; and (3) a VL CDR3 having the amino acid sequence of SEQ ID NO:10; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs.

In some embodiments, provided herein are antibodies or antigen-binding fragments thereof that specifically bind IL-7Ra having a VL, wherein the VL comprises VL CDR1, CDR2 and CDR3 having the amino acid sequences of SEQ ID NOs: 8, 9, and 10, respectively, as defined by Kabat; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs. In some embodiments, provided herein are antibodies or antigen-binding fragments thereof that specifically bind IL-7Ra having a VL, wherein the VL comprises VL CDR1, CDR2 and CDR3 having the amino acid sequences of SEQ ID NOs: 8, 9, and 10, respectively, as defined by Chothia; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs.

In some embodiments, provided herein are antibodies or antigen-binding fragments thereof that specifically bind IL-7Ra comprising a VH comprising (1) a VH CDR1 having an amino acid sequence of SEQ ID NO:11 or 12; (2) a VH CDR2 having an amino acid sequence of SEQ ID NO:13 or 14; or (3) a VH CDR3 having an amino acid sequence of SEQ ID NO:15; or a variant thereof having up to about 3, about 5, about 8, about 10, about 12, or about 15 amino acid substitutions, additions, and/or deletions in the VH CDR. In some embodiments, the variant has up to about 5 amino acid substitutions, additions, and/or deletions in the VH CDR. In some embodiments, provided herein are antibodies or antigen-binding fragments thereof that specifically bind IL-7Ra comprising a VH comprising (1) a VH CDR1 having an amino acid sequence of SEQ ID NO:11 or 12; (2) a VH CDR2 having an amino acid sequence of SEQ ID NO:13 or 14; and (3) a VH CDR3 having an amino acid sequence of SEQ ID NO: 15; or a variant thereof having up to about 3, about 5, about 8, about 10, about 12, or about 15 amino acid substitutions, additions, and/or deletions in the VH CDRs. In some embodiments, the variant has up to about 5 amino acid substitutions, additions, and/or deletions in the VH CDRs.

In some embodiments, provided herein are antibodies or antigen-binding fragments thereof that specifically bind IL-7Ra having a VH, wherein the VH comprises VH CDR1, CDR2 and CDR3 having the amino acid sequences of SEQ ID NOs: 12, 14 and 15, respectively, as defined by Kabat; or a variant thereof having up to about 3, about 5, about 8, about 10, about 12, or about 15 amino acid substitutions, additions, and/or deletions in the VH CDRs. In some embodiments, the variant has up to about 5 amino acid substitutions, additions, and/or deletions in the VH CDRs. In some embodiments, provided herein are antibodies or antigen-binding fragments thereof that specifically bind IL-7Ra having a VH, wherein the VH comprises VH CDR1, CDR2 and CDR3 having the amino acid sequences of SEQ ID NOs: 11, 13, and 15, respectively; as defined by Chothia; or a variant thereof having up to about 3, about 5, about 8, about 10, about 12, or about 15 amino acid substitutions, additions, and/or deletions in the VH CDRs. In some embodiments, the variant has up to about 5 amino acid substitutions, additions, and/or deletions in the VH CDRs.

In some embodiments, provided herein are antibodies or antigen-binding fragments thereof that specifically bind IL-7Ra, comprising, as defined by Kabat, (a) a VL comprising VL CDR1, VL CDR2, and VL CDR3 having the amino acid sequences of SEQ ID NOs: 8, 9, and 10, respectively; or a variant thereof having up to about 3, about 5, about 8, about 10, about 12, or about 15 amino acid substitutions, additions, and/or deletions in the VL CDRs; and/or (b) a VH comprising VH CDR1, VH CDR2, and VH CDR3 having the amino acid sequences of SEQ ID NOs: 12, 14 and 15, respectively; or a variant thereof having up to about 3, about 5, about 8, about 10, about 12, or about 15 amino acid substitutions, additions, and/or deletions in the VH CDRs.

In some embodiments, provided herein are antibodies or antigen-binding fragments thereof that specifically bind IL-7Ra comprising VL CDR1, VL CDR2, VL CDR3, VH CDR1, VH CDR2 and VH CDR3, having the amino acid sequences of SEQ ID NOs: 8, 9, 10, 12, 14 and 15, respectively, as defined by Kabat, or a variant thereof having up to about 3, about 5, about 8, about 10, about 12, or about 15 amino acid substitutions, additions, and/or deletions in the CDRs.

In some embodiments, provided herein are antibodies or antigen-binding fragments thereof that specifically bind IL-7Ra, comprising, as defined by Chothia (a) a VL comprising VL CDR1, VL CDR2, and VL CDR3 having the amino acid sequences of SEQ ID NOs: 8, 9, and 10, respectively; or a variant thereof having up to about 3, about 5, about 8, about 10, about 12, or about 15 amino acid substitutions, additions, and/or deletions in the VL CDRs; and/or (b) a VH comprising VH CDR1, VH CDR2, and VH CDR3 having the amino acid sequences of SEQ ID NOs: 11, 13, and 15, respectively; or a variant thereof having up to about 3, about 5, about 8, about 10, about 12, or about 15 amino acid substitutions, additions, and/or deletions in the VH CDRs.

In some embodiments, provided herein are antibodies or antigen-binding fragments thereof that specifically bind IL-7Ra comprising VL CDR1, VL CDR2, VL CDR3, VH CDR1, VH CDR2 and VH CDR3, having the amino acid sequences of SEQ ID NOs: 8, 9, 10, 11, 13, and 15, respectively, as defined by Chothia, or a variant thereof having up to about 3, about 5, about 8, about 10, about 12, or about 15 amino acid substitutions, additions, and/or deletions in the CDRs.

TABLE 3
Amino acid sequences of light chain variable region (VL) and heavy chain variable
region (VH) of chimeric antibody Ab011 (cmAb011) and the humanized VLs and VHs.

| Ab domain | Sequence |
|---|---|
| cmAb011 VL | DIVLTQSPASLAVSLGQRATISCRISESVSIHLLHLMHWYQQKPGQPPK |
| | LLIYGASNLESGVPARFSGSGSETDFTLNIHPVEEEDAATYFCQQSIKDP |
| | YTFGGGTKLEIK (SEQ ID NO: 18) |
| cmAb011 VH | QVQLQQSGAELMRPGASVTLSCKTSGYRFTDYELHWVKQTPVRGLE |
| | WIGGIDPKTGGTAYNQKFKGKAILTSDRSSSTTYMELRSLASEDSAVY |
| | YCTRSGGRWYFDVWGTGTTVTVSS (SEQ ID NO: 19) |
| humanized | DIVLTQSPASLAVSPGQRATITCRTSESVSIHLLHLMHWYQQKPGQPPK |
| VL1 | LLIYGASNLESGVPARFSGSGSGTDFTLTINPVEAEDTANYYCQQSIKD |
| | PYTFGGGTKVEIK (SEQ ID NO: 20) |
| humanized | DIVLTQSPASLAVSPGQRATITCRTSESVSIHLLHLMHWYQQKPGQPPK |
| VL2 | LLIYGASNLESGVPARFSGSGSETDFTLTINPVEAEDTANYYCQQSIKDP |
| | YTFGGGTKVEIK (SEQ ID NO: 21) |
| humanized | DIVMTQSPDSLAVSLGERATINCRTSESVSIHLLHLMHWYQQKPGQPP |
| VL3 | KLLIYGASNLESGVPDRFSGSGSGTDFTLTISSLQAEDVAVYYCQQSIK |
| | DPYTFGGGTKVEIK (SEQ ID NO: 22) |
| humanized | DIVLTQSPDSLAVSLGERATINCRTSESVSIHLLHLMHWYQQKPGQPPK |
| VL4 | LLIYGASNLESGVPDRFSGSGSETDFTLTISSLQAEDVAVYYCQQSIKDP |
| | YTFGGGTKVEIK (SEQ ID NO: 23) |
| humanized | QVQLVQSGAEVKKPGASVKVSCKASGYRFTDYELHWVRQAPGQGLE |
| VH1 | WMGGIDPKTGGTAYNQKFKGRVTMTRDTSTSTVYMELSSLRSEDTAV |
| | YYCARSGGRWYFDVWGQGTTVTVSS (SEQ ID NO: 26) |
| humanized | QVQLVQSGAEVKKPGASVKVSCKASGYRFTDYELHWVRQAPGQGLE |
| VH2 | WMGGIDPKTGGTAYNQKFKGRVTMTSDRSTSTVYMELSSLRSEDTAV |
| | YYCARSGGRWYFDVWGQGTTVTVSS (SEQ ID NO: 27) |
| humanized | QVQLVQSGAEVKKPGASVKVSCKASGYRFTDYELHWVKQAPGQGLE |
| VH3 | WIGGIDPKTGGTAYNQKFKGRVTMTSDRSTSTVYMELSSLRSEDTAVY |
| | YCARSGGRWYFDVWGQGTTVTVSS (SEQ ID NO: 28) |
| humanized | QVQLVQSGAEVKKPGASVKVSCKASGYRFTDYELHWVRQAPGQGLE |
| VH4 | WMGGIDPKTGGTAYNQKFKGKVTLTSDRSTSTVYMELSSLRSEDTAV |
| | YYCARSGGRWYFDVWGQGTTVTVSS (SEQ ID NO: 29) |
| humanized | QVQLVQSGAEVKKPGASVKVSCKASGYRFTDYELHWVKQAPGQGLE |
| VH5 | WIGGIDPKTGGTAYNQKFKGKVTLTSDRSTSTVYMELSSLRSEDTAVY |
| | YCARSGGRWYFDVWGQGTTVTVSS (SEQ ID NO: 30) |

TABLE 4
Humanized Antibodies (HuAb) with
specific humanized VLs and VHs.

| Humanized | humanized | humanized | humanized | humanized |
|---|---|---|---|---|
| antibody | VL1 | VL2 | VL3 | VL4 |
| humanized | HuAb001 | HuAb002 | HuAb003 | HuAb004 |
| VH1 | | | | |
| humanized | HuAb005 | HuAb006 | HuAb007 | HuAb008 |
| VH2 | | | | |
| humanized | HuAb009 | HuAb010 | HuAb011 | HuAb012 |
| VH3 | | | | |
| humanized | HuAb013 | HuAb014 | HuAb015 | HuAb016 |
| VH4 | | | | |
| humanized | HuAb017 | HuAb018 | HuAb019 | HuAb020 |
| VH5 | | | | |

In some embodiments, provided herein are antibodies or antigen-binding fragments thereof that specifically bind IL-7Ra comprising a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:18. In some embodiments, provided herein are antibodies or antigen-binding fragments thereof that specifically bind IL-7Ra comprising a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 19.

In some embodiments, provided herein are antibodies or antigen-binding fragments thereof that specifically bind IL-7Ra comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 18; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:19. In some embodiments, provided herein are antibodies or antigen-binding fragments thereof that specifically bind IL-7Ra comprising a VL and a VH, wherein the VL and VH have the amino acid sequences of SEQ ID NOs: 18 and 19, respectively.

In some embodiments, provided herein are antibodies or antigen-binding fragments thereof that specifically bind IL-7Ra comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:18. In some embodiments, the anti-IL-7Ra antibody or antigen-binding fragment thereof has a VL having at least 85% sequence identity to SEQ ID NO:18. In some embodiments, the anti-IL-7Ra antibody or antigen-binding fragment thereof has a VL having at least 90% sequence identity to SEQ ID NO:18. In some embodiments, the anti-IL-7Ra antibody or antigen-binding fragment thereof has a VL having at least 95% sequence identity to SEQ ID NO:18. In some embodiments, the anti-IL-7Ra antibody or antigen-binding fragment thereof has a VL having at least 98% sequence identity to SEQ ID NO:18. In some embodiments, provided herein are antibodies or antigen-binding fragments thereof that specifically bind IL-7Ra comprising a VL having the amino acid sequence of SEQ ID NO:18.

In some embodiments, provided herein are antibodies or antigen-binding fragments thereof that specifically bind IL-7Ra comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:19. In some embodiments, the anti-IL-7Ra antibody or antigen-binding fragment thereof has a VH having at least 85% sequence identity to SEQ ID NO:19. In some embodiments, the anti-IL-7Ra antibody or antigen-binding fragment thereof has a VH having at least 90% sequence identity to SEQ ID NO:19. In some embodiments, the anti-IL-7Ra antibody or antigen-binding fragment thereof has a VH having at least 95% sequence identity to SEQ ID NO:19. In some embodiments, the anti-IL-7Ra antibody or antigen-binding fragment thereof has a VH having at least 98% sequence identity to SEQ ID NO:19. In some embodiments, provided herein are antibodies or antigen-binding fragments thereof that specifically bind IL-7Ra comprising a VH having the amino acid sequence of SEQ ID NO:19.

In some embodiments, provided herein are humanized antibodies of cmAb011 (i.e., humanized Ab011, hu-cmAb011, hu-Ab011, or HuAb). In some embodiments, the humanized anti-IL-7Ra antibody or antigen-binding fragment thereof provided herein comprises a VL having an amino acid sequence selected from the group consisting of SEQ ID NOs: 20-23. In some embodiments, the humanized anti-IL-7Ra antibody or antigen-binding fragment thereof provided herein comprises a VH having an amino acid sequence selected from the group consisting of SEQ ID NOs: 26-30. In some embodiments, the humanized anti-IL-7Ra antibody or antigen-binding fragment thereof provided herein comprises a VL having an amino acid sequence selected from the group consisting of SEQ ID NOs: 20-23 and a VH having an amino acid sequence selected from the group consisting of SEQ ID NOs: 26-30. In some embodiments, the anti-IL-7Ra antibody or antigen-binding fragment thereof provided herein is a variant of a humanized Ab011 provided herein. The variant can have a VL that is a variant of the VL of a humanized Ab011 having up to about 3, about 5, about 8, about 10, about 12, or about 15 amino acid substitutions, additions, and/or deletions in an amino acid sequence selected from the group consisting of SEQ ID NOs: 20-23. The variant can have a VL that is a variant of the VL of a humanized Ab011 having up to about 5 amino acid substitutions, additions, and/or deletions in an amino acid sequence selected from the group consisting of SEQ ID NOs: 20-23. The variant can have a VH that is a variant of the VH of a humanized Ab011 having up to about 3, about 5, about 8, about 10, about 12, or about 15 amino acid substitutions, additions, and/or deletions in an amino acid sequence selected from the group consisting of SEQ ID NOs: 26-30. The variant can have a VH that is a variant of the VH of a humanized Ab011 having up to about 5 amino acid substitutions, additions, and/or deletions in an amino acid sequence selected from the group consisting of SEQ ID NOs: 26-30. In some embodiments, the variant of a humanized Ab011 has up to about 5 conservative amino acid substitutions.

In some embodiments, provided herein are humanized antibodies or antigen-binding fragments thereof that specifically bind IL-7Ra comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 20-23; and/or (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 26-30.

In some embodiments, provided herein are antibodies or antigen-binding fragments thereof that specifically bind IL-7Ra comprising a VL and a VH, wherein the VL and VH can have the amino acid sequences of SEQ ID NOs: 20 and 26, respectively. In some embodiments, the VL and VH can have the amino acid sequences of SEQ ID NOs: 20 and 27, respectively. In some embodiments, the VL and VH can have the amino acid sequences of SEQ ID NOs: 20 and 28, respectively. In some embodiments, the VL and VH can have the amino acid sequences of SEQ ID NOs: 20 and 29, respectively. In some embodiments, the VL and VH can have the amino acid sequences of SEQ ID NOs: 20 and 30, respectively. In some embodiments, the VL and VH can have the amino acid sequences of SEQ ID NOs: 21 and 26, respectively. In some embodiments, the VL and VH can have the amino acid sequences of SEQ ID NOs: 21 and 27, respectively. In some embodiments, the VL and VH can have the amino acid sequences of SEQ ID NOs: 21 and 28, respectively. In some embodiments, the VL and VH can have the amino acid sequences of SEQ ID NOs: 21 and 29, respectively. In some embodiments, the VL and VH can have the amino acid sequences of SEQ ID NOs: 21 and 30, respectively. In some embodiments, the VL and VH can have the amino acid sequences of SEQ ID NOs: 22 and 26, respectively. In some embodiments, the VL and VH can have the amino acid sequences of SEQ ID NOs: 22 and 27, respectively. In some embodiments, the VL and VH can have the amino acid sequences of SEQ ID NOs: 22 and 28, respectively. In some embodiments, the VL and VH can have the amino acid sequences of SEQ ID NOs: 22 and 29, respectively. In some embodiments, the VL and VH can have the amino acid sequences of SEQ ID NOs: 22 and 30, respectively. In some embodiments, the VL and VH can have the amino acid sequences of SEQ ID NOs: 23 and 26, respectively. In some embodiments, the VL and VH can have the amino acid sequences of SEQ ID NOs: 23 and 27, respectively. In some embodiments, the VL and VH can have the amino acid sequences of SEQ ID NOs: 23 and 28, respectively. In some embodiments, the VL and VH can have the amino acid sequences of SEQ ID NOs: 23 and 29, respectively. In some embodiments, the VL and VH can have the amino acid sequences of SEQ ID NOs: 23 and 30, respectively.

In some embodiments, provided herein are humanized antibodies or antigen-binding fragments thereof that specifically bind IL-7Ra comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:20. The humanized anti-IL-7Ra antibody or antigen-binding fragment thereof can have a VL having at least 85% sequence identity to SEQ ID NO:20. The humanized anti-IL-7Ra antibody or antigen-binding fragment thereof can have a VL having at least 90% sequence identity to SEQ ID NO:20. The humanized anti-IL-7Ra antibody or antigen-binding fragment thereof can have a VL having at least 95% sequence identity to SEQ ID NO:20. The humanized anti-IL-7Ra antibody or antigen-binding fragment thereof can have a VL having at least 98% sequence identity to SEQ ID NO:20. In some embodiments, provided herein are humanized antibodies or antigen-binding fragments thereof that specifically bind IL-7Ra comprising a VL having the amino acid sequence of SEQ ID NO:20.

In some embodiments, provided herein are humanized antibodies or antigen-binding fragments thereof that specifically bind IL-7Ra comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:21. The humanized anti-IL-7Ra antibody or antigen-binding fragment thereof can have a VL having at least 85% sequence identity to SEQ ID NO:21. The humanized anti-IL-7Ra antibody or antigen-binding fragment thereof can have a VL having at least 90% sequence identity to SEQ ID NO:21. The humanized anti-IL-7Ra antibody or antigen-binding fragment thereof can have a VL having at least 95% sequence identity to SEQ ID NO:21. The humanized anti-IL-7Ra antibody or antigen-binding fragment thereof can have a VL having at least 98% sequence identity to SEQ ID NO:21. In some embodiments, provided herein are humanized antibodies or antigen-binding fragments thereof that specifically bind IL-7Ra comprising a VL having the amino acid sequence of SEQ ID NO:21.

In some embodiments, provided herein are humanized antibodies or antigen-binding fragments thereof that specifically bind IL-7Ra comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:22. The humanized anti-IL-7Ra antibody or antigen-binding fragment thereof can have a VL having at least 85% sequence identity to SEQ ID NO:22. The humanized anti-IL-7Ra antibody or antigen-binding fragment thereof can have a VL having at least 90% sequence identity to SEQ ID NO:22. The humanized anti-IL-7Ra antibody or antigen-binding fragment thereof can have a VL having at least 95% sequence identity to SEQ ID NO:22. The humanized anti-IL-7Ra antibody or antigen-binding fragment thereof can have a VL having at least 98% sequence identity to SEQ ID NO:22. In some embodiments, provided herein are humanized antibodies or antigen-binding fragments thereof that specifically bind IL-7Ra comprising a VL having the amino acid sequence of SEQ ID NO:22.

In some embodiments, provided herein are humanized antibodies or antigen-binding fragments thereof that specifically bind IL-7Ra comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:23. The humanized anti-IL-7Ra antibody or antigen-binding fragment thereof can have a VL having at least 85% sequence identity to SEQ ID NO:23. The humanized anti-IL-7Ra antibody or antigen-binding fragment thereof can have a VL having at least 90% sequence identity to SEQ ID NO:23. The humanized anti-IL-7Ra antibody or antigen-binding fragment thereof can have a VL having at least 95% sequence identity to SEQ ID NO:23. The humanized anti-IL-7Ra antibody or antigen-binding fragment thereof can have a VL having at least 98% sequence identity to SEQ ID NO:23. In some embodiments, provided herein are humanized antibodies or antigen-binding fragments thereof that specifically bind IL-7Ra comprising a VL having the amino acid sequence of SEQ ID NO:23.

In some embodiments, provided herein are humanized antibodies or antigen-binding fragments thereof that specifically bind IL-7Ra comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:26. The humanized anti-IL-7Ra antibody or antigen-binding fragment thereof can have a VH having at least 85% sequence identity to SEQ ID NO:26. The humanized anti-IL-7Ra antibody or antigen-binding fragment thereof can have a VH having at least 90% sequence identity to SEQ ID NO:26. The humanized anti-IL-7Ra antibody or antigen-binding fragment thereof can have a VH having at least 95% sequence identity to SEQ ID NO:26. The humanized anti-IL-7Ra antibody or antigen-binding fragment thereof can have a VH having at least 98% sequence identity to SEQ ID NO:26. In some embodiments, provided herein are humanized antibodies or antigen-binding fragments thereof that specifically bind IL-7Ra comprising a VH having the amino acid sequence of SEQ ID NO:26.

In some embodiments, provided herein are humanized antibodies or antigen-binding fragments thereof that specifically bind IL-7Ra comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:27. The humanized anti-IL-7Ra antibody or antigen-binding fragment thereof can have a VH having at least 85% sequence identity to SEQ ID NO:27. The humanized anti-IL-7Ra antibody or antigen-binding fragment thereof can have a VH having at least 90% sequence identity to SEQ ID NO:27. The humanized anti-IL-7Ra antibody or antigen-binding fragment thereof can have a VH having at least 95% sequence identity to SEQ ID NO:27. The humanized anti-IL-7Ra antibody or antigen-binding fragment thereof can have a VH having at least 98% sequence identity to SEQ ID NO:27. In some embodiments, provided herein are humanized antibodies or antigen-binding fragments thereof that specifically bind IL-7Ra comprising a VH having the amino acid sequence of SEQ ID NO:27.

In some embodiments, provided herein are humanized antibodies or antigen-binding fragments thereof that specifically bind IL-7Ra comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:28. The humanized anti-IL-7Ra antibody or antigen-binding fragment thereof can have a VH having at least 85% sequence identity to SEQ ID NO:28. The humanized anti-IL-7Ra antibody or antigen-binding fragment thereof can have a VH having at least 90% sequence identity to SEQ ID NO:28. The humanized anti-IL-7Ra antibody or antigen-binding fragment thereof can have a VH having at least 95% sequence identity to SEQ ID NO:28. The humanized anti-IL-7Ra antibody or antigen-binding fragment thereof can have a VH having at least 98% sequence identity to SEQ ID NO:28. In some embodiments, provided herein are humanized antibodies or antigen-binding fragments thereof that specifically bind IL-7Ra comprising a VH having the amino acid sequence of SEQ ID NO:28.

In some embodiments, provided herein are humanized antibodies or antigen-binding fragments thereof that specifically bind IL-7Ra comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:29. The humanized anti-IL-7Ra antibody or antigen-binding fragment thereof can have a VH having at least 85% sequence identity to SEQ ID NO:29. The humanized anti-IL-7Ra antibody or antigen-binding fragment thereof can have a VH having at least 90% sequence identity to SEQ ID NO:29. The humanized anti-IL-7Ra antibody or antigen-binding fragment thereof can have a VH having at least 95% sequence identity to SEQ ID NO:29. The humanized anti-IL-7Ra antibody or antigen-binding fragment thereof can have a VH having at least 98% sequence identity to SEQ ID NO:29. In some embodiments, provided herein are humanized antibodies or antigen-binding fragments thereof that specifically bind IL-7Ra comprising a VH having the amino acid sequence of SEQ ID NO:29.

In some embodiments, provided herein are humanized antibodies or antigen-binding fragments thereof that specifically bind IL-7Ra comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:30. The humanized anti-IL-7Ra antibody or antigen-binding fragment thereof can have a VH having at least 85% sequence identity to SEQ ID NO:30. The humanized anti-IL-7Ra antibody or antigen-binding fragment thereof can have a VH having at least 90% sequence identity to SEQ ID NO:30. The humanized anti-IL-7Ra antibody or antigen-binding fragment thereof can have a VH having at least 95% sequence identity to SEQ ID NO:30. The humanized anti-IL-7Ra antibody or antigen-binding fragment thereof can have a VH having at least 98% sequence identity to SEQ ID NO:30. In some embodiments, provided herein are humanized antibodies or antigen-binding fragments thereof that specifically bind IL-7Ra comprising a VH having the amino acid sequence of SEQ ID NO:30.

In some embodiments, provided herein are anti-IL-7Ra antibodies or antigen-binding fragments thereof that comprise VL CDRs from a VL described herein (SEQ ID NO:18, 20, 21, 22, or 23), and/or VH CDRs from a VH described herein (SEQ ID NO: 19, 26, 27, 28, 29, or 30). Methods to identify CDRs are well known in the art. For example, software programs (e.g., abYsis) on publicly available websites are known to those of skill in the art for analysis of antibody sequence and determination of CDRs.

In some embodiments, provided herein are antibodies or antigen-binding fragments thereof that specifically bind IL-7Ra comprising (a) a VL comprising VL CDRs 1, 2, and 3 from a VL having an amino acid sequence of SEQ ID NO: 18; and/or (b) a VH comprising VH CDRs 1, 2, and 3 from a VH having an amino acid sequence of SEQ ID NO:19.

In some embodiments, provided herein are antibodies or antigen-binding fragments thereof that specifically bind IL-7Ra comprising (a) a VL comprising VL CDRs 1, 2, and 3 from a VL having an amino acid sequence selected from the group consisting of SEQ ID NOs: 20-23; and/or (b) a VH comprising VH CDRs 1, 2, and 3 from a VH having an amino acid sequence selected from the group consisting of SEQ ID NOs: 26-30.

In some embodiments, provided herein are antibodies or antigen-binding fragments thereof that specifically bind IL-7Ra comprising a VL, wherein the VL comprises VL CDRs 1, 2, and 3 from a VL having the amino acid sequence of SEQ ID NO: 18. In some embodiments, provided herein are antibodies or antigen-binding fragments thereof that specifically bind IL-7Ra comprising a VL, wherein the VL comprises VL CDRs 1, 2, and 3 from a VL having the amino acid sequence of SEQ ID NO: 20. In some embodiments, provided herein are antibodies or antigen-binding fragments thereof that specifically bind IL-7Ra comprising a VL, wherein the VL comprises VL CDRs 1, 2, and 3 from a VL having the amino acid sequence of SEQ ID NO:21. In some embodiments, provided herein are antibodies or antigen-binding fragments thereof that specifically bind IL-7Ra comprising a VL, wherein the VL comprises VL CDRs 1, 2, and 3 from a VL having the amino acid sequence of SEQ ID NO:22. In some embodiments, provided herein are antibodies or antigen-binding fragments thereof that specifically bind IL-7Ra comprising a VL, wherein the VL comprises VL CDRs 1, 2, and 3 from a VL having the amino acid sequence of SEQ ID NO:23.

In some embodiments, provided herein are humanized antibodies or antigen-binding fragments thereof that specifically bind IL-7Ra comprising a VH, wherein the VH comprises VH CDRs 1, 2, and 3 from a VH having the amino acid sequence of SEQ ID NO: 19. In some embodiments, provided herein are antibodies or antigen-binding fragments thereof that specifically bind IL-7Ra comprising a VH, wherein the VH comprises VH CDRs 1, 2, and 3 from a VH having the amino acid sequence of SEQ ID NO:26. In some embodiments, provided herein are antibodies or antigen-binding fragments thereof that specifically bind IL-7Ra comprising a VH, wherein the VH comprises VH CDRs 1, 2, and 3 from a VH having the amino acid sequence of SEQ ID NO:27. In some embodiments, provided herein are antibodies or antigen-binding fragments thereof that specifically bind IL-7Ra comprising a VH, wherein the VH comprises VH CDRs 1, 2, and 3 from a VH having the amino acid sequence of SEQ ID NO:28. In some embodiments, provided herein are antibodies or antigen-binding fragments thereof that specifically bind IL-7Ra comprising a VH, wherein the VH comprises VH CDRs 1, 2, and 3 from a VH having the amino acid sequence of SEQ ID NO: 29. In some embodiments, provided herein are antibodies or antigen-binding fragments thereof that specifically bind IL-7Ra comprising a VH, wherein the VH comprises VH CDRs 1, 2, and 3 from a VH having the amino acid sequence of SEQ ID NO:30.

The anti-IL-7Ra antibodies or antigen-binding fragments thereof can comprise a combination of any VL disclosed herein and any VH disclosed herein. In some embodiments, the VL and VH are connected by a linker. The linker can be a flexible linker or a rigid linker. In some embodiments, the linker has the amino acid sequence of (GGGGS)n, n=1, 2, 3, 4, or 5 (SEQ ID NO: 40). In some embodiments, the linker has the amino acid sequence of (EAAAK)n, n=1, 2, 3, 4, or 5 (SEQ ID NO:41). In some embodiments, the linker has the amino acid sequence of (PA)nPAP, n=0, 1, 2, 3, or 4 (SEQ ID NO:42).

In some embodiments, anti-IL-7Ra antibodies provided herein are IgA, IgD, IgE, IgG, or IgM antibodies. In some embodiments, the antibody is an IgA antibody. In some embodiments, the antibody is an IgD antibody. In some embodiments, the antibody is an IgE antibody. In some embodiments, the antibody is an IgG antibody. In some embodiments, the antibody is an IgM antibody. In some embodiments, the antibodies provided herein can be an IgG1 antibody, an IgG2 antibody, an IgG3 antibody, or an IgG4 antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, anti-IL-7Ra antibodies provided herein comprise a light chain and a heavy chain. The light chain can comprise a light chain constant domain (CL) and a light chain variable domain (VL). The heavy chain can comprise a heavy chain variable domain (VH) and a heavy chain constant domain (CH). The VL/VH can be any VL/VH disclosed herein. In some embodiments, the light chain constant region (CL) is kappa CL (Cκ; SEQ ID NO:33). In some embodiments, the light chain constant region (CL) is lambda CL (Cλ; SEQ ID NO:34). In some embodiments, the heavy chain can comprise a heavy chain constant domain (CH) from human IgA. In some embodiments, the heavy chain can comprise a heavy chain constant domain (CH) from human IgD. In some embodiments, the heavy chain can comprise a heavy chain constant domain (CH) from human IgE. In some embodiments, the heavy chain can comprise a heavy chain constant domain (CH) from human IgG. In some embodiments, the heavy chain can comprise a heavy chain constant domain (CH) from human IgM. In some embodiments, the heavy chain can comprise a heavy chain constant domain (CH) from human IgG1 (e.g., SEQ ID NO:35). In some embodiments, the heavy chain can comprise a heavy chain constant domain (CH) from human IgG2 (e.g., SEQ ID NO: 36). In some embodiments, the heavy chain can comprise a heavy chain constant domain (CH) from human IgG3 (e.g., SEQ ID NO:37). In some embodiments, the heavy chain can comprise a heavy chain constant domain (CH) from human IgG4 (e.g., SEQ ID NO:38). The CH can further include a C-terminal lysine (K). Expressly contemplated here are any and all combinations of the VL/VH pairs disclosed herein that specifically bind IL-7Ra (e.g., human IL-7Ra) and the CL/CH disclosed herein or otherwise known in the art.

| Light Chain | |
|---|---|
| Constant Region | Sequences |
| kappa CL (Cκ) | RTVAAPSVFIFPPSDEQLKSGTASVVCLLNNFY |
| | PREAKVQWKVDNALQSGNSQESVTEQDSKDSTY |
| | SLSSTLTLSKADYEKHKVYACEVTHQGLSSPVT |
| | KSFNRGEC |
| | (SEQ ID NO: 33) |
| lambda CL (Cλ) | GQPKAAPSVTLFPPSSEELQANKATLVCLISDF |
| | YPGAVTVAWKADSSPVKAGVETTTPSKQSNNKY |
| | AASSYLSLTPEQWKSHRSYSCQVTHEGSTVEKT |
| | VAPTECS |
| | (SEQ ID NO: 34) |

| Heavy Chain | |
|---|---|
| Constant Region | Sequences |
| IgG1 CH | ASTKGPSVFPLAPSSKSTSGGTAALGCLVKDYF |
| | PEPVTVSWNSGALTSGVHTFPAVLQSSGLYSLS |
| | SVVTVPSSSLGTQTYICNVNHKPSNTKVDKKVE |
| | PKSCDKTHTCPPCPAPELLGGPSVFLFPPKPKD |
| | TLMISRTPEVTCVVVDVSHEDPEVKFNWYVDGV |
| | EVHNAKTKPREEQYNSTYRVVSVLTVLHQDWLN |
| | GKEYKCKVSNKALPAPIEKTISKAKGQPREPQV |
| | YTLPPSRDELTKNQVSLTCLVKGFYPSDIAVEW |
| | ESNGQPENNYKTTPPVLDSDGSFFLYSKLTVDK |
| | SRWQQGNVFSCSVMHEALHNHYTQKSLSLSPG |
| | (SEQ ID NO: 35) |
| IgG2 CH | ASTKGPSVFPLAPCSRSTSESTAALGCLVKDYF |
| | PEPVTVSWNSGALTSGVHTFPAVLQSSGLYSLS |
| | SVVTVPSSNFGTQTYTCNVDHKPSNTKVDKTVE |
| | RKCCVECPPCPAPPVAGPSVFLFPPKPKDTLMI |
| | SRTPEVTCVVVDVSHEDPEVQFNWYVDGVEVHN |
| | AKTKPREEQFNSTFRVVSVLTVVHQDWLNGKEY |
| | KCKVSNKGLPAPIEKTISKTKGQPREPQVYTLP |
| | PSREEMTKNQVSLTCLVKGFYPSDISVEWESNG |
| | QPENNYKTTPPMLDSDGSFFLYSKLTVDKSRWQ |
| | QGNVFSCSVMHEALHNHYTQKSLSLSPG |
| | (SEQ ID NO: 36) |
| IgG3 CH | ASTKGPSVFPLAPCSRSTSGGTAALGCLVKDYF |
| | PEPVTVSWNSGALTSGVHTFPAVLQSSGLYSLS |
| | SVVTVPSSSLGTQTYTCNVNHKPSNTKVDKRVE |
| | LKTPLGDTTHTCPRCPEPKSCDTPPPCPRCPEP |
| | KSCDTPPPCPRCPEPKSCDTPPPCPRCPAPELL |
| | GGPSVFLFPPKPKDTLMISRTPEVTCVVVDVSH |
| | EDPEVQFKWYVDGVEVHNAKTKPREEQYNSTFR |
| | VVSVLTVLHQDWLNGKEYKCKVSNKALPAPIEK |
| | TISKTKGQPREPQVYTLPPSREEMTKNQVSLTC |
| | LVKGFYPSDIAVEWESSGQPENNYNTTPPMLDS |
| | DGSFFLYSKLTVDKSRWQQGNIFSCSVMHEALH |
| | NRFTQKSLSLSPG |
| | (SEQ ID NO: 37) |
| IgG4 CH | ASTKGPSVFPLAPCSRSTSESTAALGCLVKDYF |
| | PEPVTVSWNSGALTSGVHTFPAVLQSSGLYSLS |
| | SVVTVPSSSLGTKTYTCNVDHKPSNTKVDKRVE |
| | SKYGPPCPSCPAPEFLGGPSVFLFPPKPKDTLM |
| | ISRTPEVTCVVVDVSQEDPEVQFNWYVDGVEVH |
| | NAKTKPREEQFNSTYRVVSVLTVLHQDWLNGKE |
| | YKCKVSNKGLPSSIEKTISKAKGQPREPQVYTL |
| | PPSQEEMTKNQVSLTCLVKGFYPSDIAVEWESN |
| | GQPENNYKTTPPVLDSDGSFFLYSRLTVDKSRW |
| | QEGNVFSCSVMHEALHNHYTQKSLSLSLG |
| | (SEQ ID NO: 38) |

In some embodiments, the antibodies provided herein have a light chain constant region (CL) having at least 85%, at least 90%, at least 95%, at least 98%, or at least 99% sequence identity to SEQ ID NO:33. In some embodiments, the antibodies provided herein have a CL having the amino acid sequence of SEQ ID NO:33. In some embodiments, the antibodies provided herein have a light chain constant region (CL) having at least 85%, at least 90%, at least 95%, at least 98%, or at least 99% sequence identity to SEQ ID NO:34. In some embodiments, the antibodies provided herein have a CL having the amino acid sequence of SEQ ID NO:34. In some embodiments, the antibodies provided herein have a heavy chain constant region (CH) having at least 85%, at least 90%, at least 95%, at least 98%, or at least 99% sequence identity to SEQ ID NO:35. In some embodiments, the antibodies provided herein have a CH having the amino acid sequence of SEQ ID NO:35. In some embodiments, the antibodies provided herein have a CH having at least 85%, at least 90%, at least 95%, at least 98%, or at least 99% sequence identity to SEQ ID NO:36. In some embodiments, the antibodies provided herein have a CH having the amino acid sequence of SEQ ID NO:36. In some embodiments, the antibodies provided herein have a CH having at least 85%, at least 90%, at least 95%, at least 98%, or at least 99% sequence identity to SEQ ID NO:37. In some embodiments, the antibodies provided herein have a CH having the amino acid sequence of SEQ ID NO:37. In some embodiments, the antibodies provided herein have a CH having at least 85%, at least 90%, at least 95%, at least 98%, or at least 99% sequence identity to SEQ ID NO:38. In some embodiments, the antibodies provided herein have a CH having the amino acid sequence of SEQ ID NO:38.

In some embodiments, provided herein are also antibodies or antigen-binding fragments that compete with the antibody or antigen-binding fragment provided above (e.g., cmAb011) for binding to IL-7Ra (e.g., human IL-7Ra). Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments, e.g., BIACORE® surface plasmon resonance (SPR) analysis or flow cytometry. In some embodiments, an anti-IL-7Ra antibody or antigen-binding fragment competes with, and inhibits binding of another antibody or antigen-binding fragment to IL-7Ra by at least 50%, 60%, 70%, 80%, 90% or 100%. Competition assays can be conducted as described, for example, in Ed Harlow and David Lane, Cold Spring Harb Protoc; 2006; doi: 10.H01/pdb.prot4277 or in Chapter 11 of “Using Antibodies” by Ed Harlow and David Lane, Cold Spring Harbor Laboratory Press, Cold Spring Harbor, NY, USA 1999.

In some embodiments, provided herein are antibodies or antigen-binding fragments that compete with an anti-IL-7Ra antibody or antigen-binding fragment disclosed herein for binding to IL-7Ra (e.g., human IL-7Ra). In some embodiments, provided herein are antibodies or antigen-binding fragments that compete with chimeric Ab011 for binding to IL-7Ra (e.g., human IL-7Ra). In some embodiments, provided herein are antibodies or antigen-binding fragments that compete with a humanized Ab011 disclosed herein for binding to IL-7Ra (e.g., human IL-7Ra).

Epitope mapping is a method of identifying the binding site, region, or epitope on a target protein where an antibody binds. A variety of methods are known in the art for mapping epitopes on target proteins. These methods include mutagenesis, including but not limited to, shotgun mutagenesis, site-directed mutagenesis, and alanine scanning; domain or fragment scanning; peptide scanning (e.g., Pepscan technology); display methods (e.g., phage display, microbial display, and ribosome/mRNA display); methods involving proteolysis and mass spectroscopy; and structural determination (e.g., X-ray crystallography and NMR). In some embodiments, anti-IL-7Ra antibodies or antigen-binding fragments described herein are characterized by assays including, but not limited to, N-terminal sequencing, amino acid analysis, HPLC, mass spectrometry, ion exchange chromatography, and papain digestion.

The anti-IL-7Ra antibodies or antigen-binding fragments of the present disclosure can be analyzed for their physical, chemical and/or biological properties by various methods known in the art. In some embodiments, an anti-IL-7Ra antibody is tested for its ability to bind IL-7Ra (e.g., human IL-7Ra). Binding assays include, but are not limited to, BLI, SPR (e.g., Biacore), ELISA, and FACS. In addition, antibodies can be evaluated for solubility, stability, thermostability, viscosity, expression levels, expression quality, and/or purification efficiency.

In some embodiments, anti-IL-7Ra antibodies or antigen-binding fragments described herein bind to human IL-7Ra with high affinity, for example, with a K_{D} of 10⁻⁷ M or less, 5×10⁻⁸ M or less, 10⁻⁸ M or less, 5×10⁻⁹ M or less, 10⁻⁹ M or less, 5×10⁻¹⁰ M or less, or 10⁻¹⁰ M or less. In some embodiments, anti-IL-7Ra antibodies or antigen-binding fragments described herein bind to human IL-7Ra with a K_{D} of 10⁻⁹ M or less. In some embodiments, anti-IL-7Ra antibodies or antigen-binding fragments described herein bind to human IL-7Ra with high affinity, for example, with a K_{D} of about 10⁻⁷ M, about 5×10⁻⁸ M, about 10⁻⁸ M, about 5×10⁻⁹ M, about 10⁻⁹ M, about 5×10⁻¹⁰ M, or about 10⁻¹⁰ M. In some embodiments, anti-IL-7Ra antibodies or antigen-binding fragments described herein bind to human IL-7Ra with a K_{D} of about 10⁻⁹ M. In some embodiments, anti-IL-7Ra antibodies or antigen-binding fragments described herein bind to human IL-7Ra with a K_{D} ranging from 10⁻¹⁰ M to 10⁻⁷ M, from 10⁻⁹ M to 10⁻⁷ M, from 10⁻⁸ M to 10⁻⁷ M, from 10⁻¹⁰ M to 5×10⁻⁸ M, from 10⁻⁹ M to 5×10⁻⁸ M, from 10⁻⁸ M to 5×10⁻⁸ M, from 10⁻¹⁰ M to 10⁻⁸ M, from 10⁻⁹ M to 10⁻⁸ M, from 10⁻¹⁰ M to 5×10⁻⁹ M, from 10⁻⁹ M to 5×10⁻⁹ M, or from 10⁻¹⁰ M to 10⁻⁹ M. In some embodiments, anti-IL-7Ra antibodies or antigen-binding fragments described herein bind to human IL-7Ra with high affinity, for example, with a K_{D} from 10⁻¹⁰ M to 10⁻⁹ M. In some embodiments, the K_{D} is determined by BLI. In some embodiments, the K_{D} is determined by SPR. In some embodiments, anti-IL-7Ra antibodies or antigen-binding fragments described herein bind to human IL-7Ra with high affinity, for example, with a K_{D} of 10⁻⁷ M or less, 5×10⁻⁸ M or less, 10⁻⁸M or less, 5×10⁻⁹ M or less, 10⁻⁹ M or less, 5×10⁻¹⁰ M or less, or 10⁻¹⁰ M or less; or about 10⁻⁷ M, about 5×10⁻⁸ M, about 10⁻⁸M, about 5×10⁻⁹ M, about 10⁻⁹ M, about 5×10⁻¹⁰ M, or about 10⁻¹⁰ M; or ranging from 10⁻¹⁰ M to 10⁻⁷ M, from 10⁻⁹ M to 10⁻⁷ M, from 10⁻⁸ M to 10⁻⁷ M, from 10⁻¹⁰ M to 5×10⁻⁸ M, from 10⁻⁹ M to 5×10⁻⁸ M, from 10⁻⁸ M to 5×10⁻⁸ M, from 10⁻¹⁰ M to 10⁻⁸ M, from 10⁻⁹ M to 10⁻⁸ M, from 10⁻¹⁰ M to 5×10⁻⁹ M, from 10⁻⁹ M to 5×10⁻⁹ M, or from 10⁻¹⁰ M to 10⁻⁹ M, as measured by SPR.

In some embodiments, the anti-IL-7Ra antibodies or antigen-binding fragments described herein specifically bind to human IL-7Ra. In some embodiments, the anti-IL-7Ra antibodies or antigen-binding fragments described herein bind to both human IL-7Ra and cynomolgus IL-7Ra.

In some embodiments, the anti-IL-7Ra antibodies or antigen-binding fragments provided herein can block both IL-7 signaling and the TSLP signaling. The terms block, inhibit, and antagonize are used herein synonymously. No term is intended to suggest the requirement of total blocking; partial inhibition—corresponding to a reduction but not complete abolition of the biological effect—is also contemplated.

In some embodiments, the anti-IL-7Ra antibodies or antigen-binding fragments provided herein can block IL-7 signaling. In some embodiments, anti-IL-7Ra antibodies or antigen-binding fragments described herein block IL-7 binding to IL-7R. In some embodiments, anti-IL-7Ra antibodies or antigen-binding fragments described herein block IL-7 binding to IL-7Ra. As known in the art, STAT5 is a key downstream component of the IL-7 signaling pathway, and its phosphorylation (pSTAT5) is a reliable indicator of pathway activation. In some embodiments, anti-IL-7Ra antibodies or antigen-binding fragments described herein block IL-7-induced STAT5 phosphorylation.

In an exemplary pSTAT-5 assay, PBMCs can be stimulated with IL-7 in the presence and absence of a test agent. Cells can be subsequently assessed quantitatively for the level of pSTAT-5, e.g., by staining for pSTAT-5 (e.g., with a labelled anti-pSTAT-5 antibody) followed by fluorescence activated cell sorting. The levels of phosphorylated STAT-5 could also be determined by ELISA. In some embodiments, an anti-IL-7Ra antibody or antigen-binding fragment disclosed herein blocks or inhibits IL-7 induced phosphorylation of STAT5 directly downstream of IL-7R. In some embodiments, the anti-IL-7Ra antibody or antigen-binding fragment can reduce levels of phosphorylated STAT-5 by at least 20%, at least 50%, at least 75%, at least 80%, at least 85%, at least 90%, at least 95% or 100% when compared to phosphorylated STAT-5 levels in its absence, or when compared to a negative control.

In some embodiments, the anti-IL-7Ra antibodies or antigen-binding fragments disclosed herein can antagonize the biological effect of IL-7, for example, IL-7-mediated T cell proliferation. In some embodiments, an anti-IL-7Ra antibody or antigen-binding fragment disclosed herein blocks or inhibits IL-7 induced T cell proliferation. In some embodiments, the anti-IL-7Ra antibody or antigen-binding fragment can reduce IL-7 induced proliferating T cells by at least 20%, at least 50%, at least 75%, at least 80%, at least 85%, at least 90%, at least 95% or 100% when compared to IL-7 induced proliferating T cells in its absence, or when compared to a negative control. In some embodiments, the anti-IL-7Ra antibodies or antigen-binding fragments described herein block IL-7-induced T cell proliferation. In some embodiments, the anti-IL-7Ra antibodies or antigen-binding fragments described herein reduce or prevent IL-7 induced autoimmunity or inflammation.

In some embodiments, the anti-IL-7Ra antibodies or antigen-binding fragments provided herein can block TSLP signaling. In some embodiments, the anti-IL-7Ra antibodies or antigen-binding fragments described herein block TSLP binding to TSLPR. In some embodiments, anti-IL-7Ra antibodies or antigen-binding fragments described herein block TSLP binding to IL-7Ra. As known in the art, Thymus and Activation-Regulated Chemokine (TARC) is a chemokine that is often produced in response to TSLP activation and commonly used as a marker to measure TSLP pathway activity. In some embodiments, an anti-IL-7Ra antibody or antigen-binding fragment disclosed herein blocks or inhibits TSLP-induced TARC secretion directly downstream of TSLPR. In an exemplary TARC secretion assay, PBMCs can be stimulated with TSLP in the presence and absence of a test agent. Cells can be subsequently assessed quantitatively for the level of TARC, e.g., by ELISA. In some embodiments, the anti-IL-7Ra antibody or antigen-binding fragment can reduce levels of TSLP-induced TARC secretion by at least 20%, at least 50%, at least 75%, at least 80%, at least 85%, at least 90%, at least 95% or 100% when compared to TARC secretion levels in its absence, or when compared to a negative control. In some embodiments, the anti-IL-7Ra antibodies or antigen-binding fragments disclosed herein can antagonize the biological effect of TSLP. In some embodiments, the anti-IL-7Ra antibodies or antigen-binding fragments described herein reduce or prevent TSLP induced autoimmunity or inflammation.

In some embodiments, the anti-IL-7Ra antibodies or antigen-binding fragments described herein ameliorate an IL-7Ra related autoimmune or inflammatory disease, such as UC.

The anti-IL-7Ra antibodies or antigen-binding fragments provided herein can have one or more of the functional properties described above. In some embodiments, the anti-IL-7Ra antibodies or antigen-binding fragments provided herein (1) bind to human IL-7Ra with high affinity; (2) block IL-7 binding to IL-7Ra; (3) block IL-7-induced STAT5 phosphorylation; (4) block IL-7-induced T cell proliferation; (5) block TSLP induced TARC secretion; or (6) ameliorate colitis; or any combination of (1)-(6).

### 7.3 Variants and Conjugates

The present disclosure further contemplates additional variants and equivalents that are substantially homologous to the recombinant, monoclonal, chimeric, humanized, and human antibodies, or antibody fragments thereof, described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, and/or solubility. Those skilled in the art will appreciate that amino acid changes may alter post-translational processes of an antibody, such as changing the number or position of glycosylation sites or altering membrane anchoring characteristics.

Variations can be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine, e.g., conservative amino acid replacements. Insertions or deletions can be in the range of about 1 to 5 amino acids. In some embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. In some embodiments, variations in the amino acid sequence that are biologically useful and/or relevant can be determined by systematically making insertions, deletions, or substitutions in the sequence and testing the resulting variant proteins for activity as compared to the parent protein.

In some embodiments, provided herein are variants of anti-IL-7Ra antibodies or antigen-binding fragments described herein. In some embodiments, provided herein are variants of anti-IL-7Ra antibody clone Ab011 (cmAb011 or HuAb). In some embodiments, a variant comprises one to 30 amino acid substitutions, additions, and/or deletions in the parent antibody or antigen-binding fragment. In some embodiments, a variant comprises one to 25 amino acid substitutions, additions, and/or deletions in the parent antibody or antigen-binding fragment. In some embodiments, a variant comprises one to 20 substitutions, additions, and/or deletions in the parent antibody or antigen-binding fragment. In some embodiments, a variant comprises one to 15 substitutions, additions, and/or deletions in the parent antibody or antigen-binding fragment. In some embodiments, a variant comprises one to 10 substitutions, additions, and/or deletions in the parent antibody or antigen-binding fragment. In some embodiments, a variant comprises one to five amino acid substitutions, additions, and/or deletions in the parent antibody or antigen-binding fragment. In some embodiments, a variant comprises one to three amino acid substitutions, additions, and/or deletions in the parent antibody or antigen-binding fragment. In some embodiments, the amino acid substitution(s) is in a CDR of the antibody or antigen-binding fragment. In some embodiments, the amino acid substitution(s) is not in a CDR of the antibody or antigen-binding fragment. In some embodiments, the amino acid substitution(s) is in a framework region of the antibody or antigen-binding fragment. In some embodiments, the amino acid substitutions, additions, and/or deletions are conservative amino acid substitutions.

It is known in the art that the constant region(s) of an antibody mediates several effector functions and these effector functions can vary depending on the isotype of the antibody. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In some embodiments, the anti-IL-7Ra antibodies or antigen-binding fragments described herein comprise a constant region of a human IgA antibody. In some embodiments, the anti-IL-7Ra antibodies or antigen-binding fragments described herein comprise a constant region of a human IgD antibody. In some embodiments, the anti-IL-7Ra antibodies or antigen-binding fragments described herein comprise a constant region of a human IgE antibody. In some embodiments, the anti-IL-7Ra antibodies or antigen-binding fragments described herein comprise a constant region of a human IgG antibody. In some embodiments, anti the anti-IL-7Ra antibodies or antigen-binding fragments described herein comprise a constant region of a human IgM antibody. In some embodiments, the anti-IL-7Ra antibodies or antigen-binding fragments described herein comprise a constant region of a human IgG1 antibody. In some embodiments, the anti-IL-7Ra antibodies or antigen-binding fragments described herein comprise a constant region of a human IgG2 antibody. In some embodiments, the anti-IL-7Ra antibodies or antigen-binding fragments described herein comprise a constant region of a human IgG3 antibody. In some embodiments, the anti-IL-7Ra antibodies or antigen-binding fragments described herein comprise a constant region of a human IgG4 antibody.

In some embodiments, at least one or more of the constant regions has been modified or deleted in the anti-IL-7Ra antibody or antigen-binding fragment described herein. In some embodiments, the antibodies comprise modifications to one or more of the three heavy chain constant regions (CH1, CH2 or CH3) and/or to the light chain constant region (CL).

In some embodiments, the heavy chain constant region of the modified antibodies comprises at least one human constant region. In some embodiments, the heavy chain constant region of the modified antibodies comprises more than one human constant region. In some embodiments, modifications to the constant region comprise additions, deletions, or substitutions of one or more amino acids in one or more regions. In some embodiments, one or more regions are partially or entirely deleted from the constant regions of the modified antibodies. In some embodiments, the entire CH2 domain has been removed from an antibody (ACH2 constructs). In some embodiments, a deleted constant region is replaced by a short amino acid spacer that provides some of the molecular flexibility typically imparted by the absent constant region. In some embodiments, a modified antibody comprises a CH3 domain directly fused to the hinge region of the antibody. In some embodiments, a modified antibody comprises a peptide spacer inserted between the hinge region and modified CH2 and/or CH3 domains.

In some embodiments, an anti-IL-7Ra antibody or antigen-binding fragment comprises a Fc region. In some embodiments, the Fc region is fused via a hinge. The hinge can be an IgG1 hinge, an IgG2 hinge, or an IgG3 hinge. The amino acid sequences of the Fc region of human IgG1, IgG2, IgG3, and IgG4 are known to those of ordinary skill in the art. In some cases, Fc regions with amino acid variations have been identified in native antibodies. In some embodiments, the modified antibodies (e.g., modified Fc region) provide for altered effector functions that, in turn, affect the biological profile of the antibody. For example, in some embodiments, the deletion or inactivation (through point mutations or other means) of a constant region reduces Fc receptor binding of the modified antibody as it circulates. In some embodiments, the constant region modifications reduce the immunogenicity of the antibody. In some embodiments, the constant region modifications increase the serum half-life of the antibody. In some embodiments, the constant region modifications reduce the serum half-life of the antibody. In some embodiments, the constant region modifications decrease or remove ADCC and/or CDC of the antibody. In some embodiments, specific amino acid substitutions in a human IgG1 Fc region with corresponding IgG2 or IgG4 residues reduce effector functions (e.g., ADCC and CDC) in the modified antibody. In some embodiments, an antibody does not have one or more effector functions (e.g., “effectorless” antibodies). In some embodiments, the antibody does not bind an Fc receptor and/or complement factors. In some embodiments, the antibody has no effector function(s). In some embodiments, the constant region is modified to eliminate disulfide linkages or oligosaccharide moieties. In some embodiments, the constant region is modified to add/substitute one or more amino acids to provide one or more oligosaccharide, or carbohydrate attachment sites. In some embodiments, an anti-IL-7Ra antibody or antigen-binding fragment comprises a variant Fc region that is engineered with substitutions at specific amino acid positions as compared to a native Fc region.

In some embodiments of the antibodies provided herein, the Fc domain comprises one or more amino acid substitution that reduces binding to an Fc receptor. The Fc receptor can be a human Fc receptor. The Fc receptor can be an Fcγ receptor. The Fc receptor can be an activating Fc receptor. The Fc receptor can be an activating human Fcγ receptor, such as a human Fcγ RIIIa, Fcγ RI or Fcγ RIIa. In some embodiments of the antibodies provided herein, the Fc domain comprises one or more amino acid substitution that reduces the effector function. In some embodiments of the antibodies provided herein, the same one or more amino acid substitution is present in each of the two subunits of the Fc region. In one aspect, the one or more amino acid substitution reduces the binding affinity of the Fc region to an Fc receptor. In one aspect, the one or more amino acid substitution reduces the binding affinity of the Fc region to an Fc receptor by at least 2-fold, at least 5-fold, or at least 10-fold.

Variants with reduced effector functions are known in the art and can be incorporated in the antibodies disclosed herein. For example, amino acid substitutions are known to reduce effector function. hIgG1 L235A/G237A/E318A antibody is unable to bind to human cell lines expressing FcγRs, resulting in reduced ADCC. hIgG1 and hIgG4 antibodies with L234A/L235A Fc domains have no detectable binding to the low affinity FcγRs and C1q and significantly reduced ADCC and CDC. Mutations at specific residues in hIgG1 known to interact with both FcγRs and C1q, such as amino acid substitutions L234F/L235E/P331S, can reduce binding to the low affinity FcγRs and result in no detectable binding to FcγRI. The G236R/L328R mutation pair reduces or completely abrogates binding to the FcγRs. S267E substitution also reduces binding for all low affinity hFcγRs. S267K substitution combined with a series of mutations in the lower hinge of hIgG2 (E233P/L234V/L235A mutations and a deletion of residue G236) and incorporated into a hIgG1 background result in a lack of binding to all hFcγR. P329G disrupts the interaction between hIgG and hFcγR. The triple mutant L234A/L235A/P329G has no detectable binding to C1q or FcγRs, resulting in abrogated ADCC when introduced into a hIgG1. Combined point mutations of N297Q, L234F, L235E, D265A, P331S ablate Fc function. The combination of L234F/L235E/D265A potently silences the Fc region, resulting in no detectable binding to FcγRI, reduced binding to the low affinity FcγRs and reduced binding to C1q. From the site saturation mutagenesis libraries centered about the Fc C′/E loop, the S298G/T299A mutations are found to abolish or significantly reduce binding to C1q and most FcγRs except for FcγRIIA-R131 and FcγRIIB.

Additionally, glycoengineering techniques can be used to generate antibodies with reduced effector functions. The N297 glycan is central to the binding between hIgG1 and FcγRs and C1q. As such, amino acid mutations at this site which remove this glycan, including N297A, N297Q and N297G, can reduce binding to all FcγRs and C1q, resulting in reduction of ADCC and CDC.

For hIgG4, which has low affinity for all FcγR, the serine at position 228 plays a pivotal role in F(ab) arm exchange. The S228P substitution can provide homogeneous hIgG4, and is commonly introduced in therapeutic hIgG4 antibodies. Based upon its inherent lack of effector function, the human γ4 constant region can be used in Fc-silencing approaches. For example, exchanging the human γ1 region with that of human γ4 can reduce effector functionality. Murine IgG2b isotype, which also has low FcγR binding activity, differs from hIgG4 at position 235. Incorporating the mouse IgG2b residue (glutamic acid) into the hIgG4 antibody at this position can further minimize Fc effector function, resulting in an antibody (with the S228P/L235E mutations) with substantially reduced, if any, binding to all FcγRs and C1q, and no measurable ADCC. Additionally, rather than replacing the whole constant region of hIgG1 with hIgG4, specific amino acids from human γ4 can be introduced into antibodies of other IgG isotypes. For example, a combination of amino acid mutations-H268Q/V309L/A330S/P331S (IgG2m4), when introduced into a hIgG2 backbone, can lead to no detectable binding to hFcγRI, hFcγRIIIA or C1q, reduced binding to hFcγRIIB and no change in binding to FcγRIIA-H131 when compared to the WT hIgG2 antibody. For another example, the V234A/G237A/P238S/H268A/V309L/A330S/P331S (IgG2c4d) mutations, where multiple residues within the hIgG2 constant region are replaced with IgG4 residues, can result in no detectable binding to any FcγRs or C1q and no measurable ADCC, ADCP or CDC when compared to the WT hIgG2 counterpart.

Accordingly, for illustrative purposes, such variants include: aglycosylation (N297A/Q/G; or “NA”), L235A/G237A/E318A (“AAA”), L234A/L235A (“LALA”), S228P/L235E (“IgG4 PE”), G236R/L328R (“RR”), S298G/T299A (“GA”), L234F/L235E/P331S (“FES”), H268Q/V309L/A330S/P331S (“IgG2m4”), E233P/L234V/L235A/deletion of G236/S267K, L234A/L235A/P329G (“LALAPG”), V234A/G237A/P238S/H268A/V309L/A330S/P331S (“IgG2c4d”), and L234F/L235E/D265A (“FEA”). (See Liu et al., Antibodies 9.4 (2020): 64; Delidakis et al., Annual review of biomedical engineering 24 (2022): 249-274, both incorporated herein by reference in their entireties). In some embodiments, the anti-IL-7Ra antibodies or antigen-binding fragments disclosed herein comprise the NA mutation. In some embodiments, the anti-IL-7Ra antibodies or antigen-binding fragments disclosed herein comprise the AAA mutations. In some embodiments, the anti-IL-7Ra antibodies or antigen-binding fragments disclosed herein comprise the LALA mutations. In some embodiments, the anti-IL-7Ra antibodies or antigen-binding fragments disclosed herein comprise the RR mutations. In some embodiments, the anti-IL-7Ra antibodies or antigen-binding fragments disclosed herein comprise the GA mutations. In some embodiments, the anti-IL-7Ra antibodies or antigen-binding fragments disclosed herein comprise the FES mutations. In some embodiments, the anti-IL-7Ra antibodies or antigen-binding fragments disclosed herein comprise the LALAPG mutations. In some embodiments, the anti-IL-7Ra antibodies or antigen-binding fragments disclosed herein comprise the FEA mutations. In some embodiments, the anti-IL-7Ra antibodies or antigen-binding fragments disclosed herein comprise the IgG2m4 mutations. In some embodiments, the anti-IL-7Ra antibodies or antigen-binding fragments disclosed herein comprise the IgG2-PE mutations. In some embodiments, the anti-IL-7Ra antibodies or antigen-binding fragments disclosed herein comprise the IgG2c4d mutations. As a person of ordinary skill in the art would understand, the anti-IL-7Ra antibodies or antigen-binding fragments disclosed herein are not limited by specific Fc modifications, and any combination and permutations of the Fc modifications disclosed herein or otherwise known in the art that reduce the effector function or binding affinity to FcγR can be adopted in anti-IL-7Ra antibodies or antigen-binding fragments.

In some embodiments, an anti-IL-7Ra antibody or antigen-binding fragment described herein comprises an IgG4 heavy chain constant region that comprises an amino acid substitution of S228, numbered according to the EU Index. In some embodiments, an anti-IL-7Ra antibody or antigen-binding fragment described herein comprises an IgG4 heavy chain constant region that comprises at least one amino acid substitution. The IgG4 heavy chain constant region can comprise an S228 substitution. The S228 substitution can be, e.g., S228P (SEQ ID NO:39). In some embodiments, provided herein are IgG4 antibodies having a heavy chain constant region (CH) having at least 85% sequence identity to an amino acid sequence of SEQ ID NO:39. In some embodiments, the IgG4 antibodies provided herein have a CH having at least 85%, at least 90%, at least 95%, at least 98%, or at least 99% sequence identity to SEQ ID NO:39. The CH can further include a C-terminal lysine (K).

In some embodiments, variants can include addition of amino acid residues at the amino- and/or carboxyl-terminal end of the antibody or polypeptide. The length of additional amino acids residues can range from one residue to a hundred or more residues. In some embodiments, a variant comprises an N-terminal methionyl residue. In some embodiments, the variant comprises an additional polypeptide/protein (e.g., Fc region) to create a fusion protein. In some embodiments, a variant is engineered to be detectable and can comprise a detectable label and/or protein (e.g., a fluorescent tag or an enzyme).

The variant antibodies or antigen-binding fragments described herein can be generated using methods known in the art, including but not limited to, site-directed mutagenesis, alanine scanning mutagenesis, and PCR mutagenesis. Methods for mutagenesis and nucleotide sequence alterations are well known in the art. See, for example, Walker and Gaastra, eds. (1983) TECHNIQUES IN MOLECULAR BIOLOGY (MacMillan Publishing Company, New York); Kunkel, Proc. Natl. Acad. Sci. USA 82:488-492 (1985); Kunkel et al., Methods Enzymol. 54:367-382 (1987); Sambrook et al. (1989) MOLECULAR CLONING: A LABORATORY MANUAL (Cold Spring Harbor, N.Y.); U.S. Pat. No. 4,873,192; and the references cited therein; herein incorporated by reference. Guidance as to appropriate amino acid substitutions that do not affect biological activity of the polypeptide of interest can be found in the model of Dayhoff et al. (1978) in Atlas of Protein Sequence and Structure (Natl. Biomed. Res. Found., Washington, D.C.), pp. 345-352, herein incorporated by reference in its entirety. The model of Dayhoff et al. uses the Point Accepted Mutation (PAM) amino acid similarity matrix (PAM 250 matrix) to determine suitable conservative amino acid substitutions. Conservative substitutions, such as exchanging one amino acid with another having similar properties, can be beneficial. Examples of conservative amino acid substitutions as taught by the PAM 250 matrix of the Dayhoff et al. model include, but are not limited to, Gly→Ala, Val→Ile→Leu, Asp→Glu, Lys→Arg, Asn→Gln, and Phe→Trp→Tyr.

In constructing variants of an anti-IL-7Ra binding molecule, e.g., an antibody or antigen-binding fragment, variant, or derivative thereof, modifications are made such that variants continue to possess the desired properties, e.g., being capable of specifically binding to an IL-7Ra, and in certain embodiments being able to block IL-7 signaling, and/or to block TSLP signaling. Obviously, any mutations made in the DNA encoding the variant polypeptide must not place the sequence out of reading frame. In some embodiments, mutations made in the DNA do not create complementary regions that could produce secondary mRNA structure.

In some embodiments, a variant of an anti-IL-7Ra antibody or antigen-binding fragment disclosed herein can retain the ability to bind IL-7Ra to a similar extent, the same extent, or to a higher extent, as the parent antibody or antigen-binding fragment. In some embodiments, the variant can be at least about 80%, about 85%, about 90%, about 91%, about 92%, about 93%, about 94%, about 95%, about 96%, about 97%, about 98%, about 99% or more identical in amino acid sequence to the parent antibody or antigen-binding fragment. In certain embodiments, a variant of an anti-IL-7Ra antibody or antigen-binding fragment comprises the amino acid sequence of the parent anti-IL-7Ra antibody or antigen-binding fragment with one or more conservative amino acid substitution. Conservative amino acid substitutions are known in the art and include amino acid substitutions in which one amino acid having certain physical and/or chemical properties is exchanged for another amino acid that has the same or similar chemical or physical properties.

In some embodiments, a variant of an anti-IL-7Ra antibody or antigen-binding fragment comprises the amino acid sequence of the parent antibody or antigen-binding fragment with one or more non-conservative amino acid substitutions. In some embodiments, a variant of an anti-IL-7Ra antibody or antigen-binding fragment comprises the amino acid sequence of the parent binding antibody or antigen-binding fragment with one or more non-conservative amino acid substitution, wherein the one or more non-conservative amino acid substitutions do not interfere with or inhibit one or more biological activities of the variant (e.g., IL-7Ra binding). In certain embodiments, the one or more conservative amino acid substitutions and/or the one or more non-conservative amino acid substitutions can enhance a biological activity of the variant, such that the biological activity of the functional variant is increased as compared to the parent antibody or antigen-binding fragment.

In some embodiments, the variant can have 1, 2, 3, 4, or 5 amino acid substitutions in the CDRs (e.g., VH CDR1, VH CDR2, VH CDR3, VL CDR1, VL CDR2 and VL CDR3) of the binding moiety.

In some embodiments, anti-IL-7Ra antibodies or antigen-binding fragments described herein are chemically modified naturally or by intervention. In some embodiments, the anti-IL-7Ra antibodies or antigen-binding fragments have been chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications can be carried out by known techniques. The anti-IL-7Ra antibodies or antigen-binding fragments can comprise one or more analogs of an amino acid (including, for example, unnatural amino acids), as well as other modifications known in the art.

In some embodiments, anti-IL-7Ra antibodies or antigen-binding fragments disclosed herein can be linked to at least one agent to form an antibody conjugate. The conjugate can be, for example, an antibody conjugated to another protein, carbohydrate, lipid, steroids, immunosuppressors, or mixed moiety molecule(s). Such antibody conjugates include, but are not limited to, modifications that include linking the antibody to one or more polymers. For example, an antibody or antigen-binding fragment can be linked to one or more water-soluble polymers. Linkage to a water-soluble polymer reduces the likelihood that the antibody or antigen-binding fragment precipitate in an aqueous environment, such as a physiological environment. One skilled in the art can select a suitable water-soluble polymer based on considerations including, but not limited to, whether the polymer/antibody conjugate will be used in the treatment of a patient and, if so, the pharmacological profile of the antibody (e.g., half-life, dosage, activity, antigenicity, and/or other factors).

In order to increase the efficacy of antibody molecules as diagnostic or therapeutic agents, it is conventional to link or covalently bind or complex at least one desired molecule or moiety. Such a molecule or moiety can be, but is not limited to, at least one effector or reporter molecule. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, an enzyme (e.g., that catalyzes a colorimetric or fluorometric or bioluminescent reaction), a substrate, a solid matrix, such as biotin. An antibody can comprise one, two, or more of any of these labels.

Antibody conjugates are also used as diagnostic agents. In some embodiments, an anti-IL-7Ra antibody or antigen-binding fragment described herein is conjugated to a detectable substance or molecule that allows the agent to be used for diagnosis and/or detection. A detectable substance can include, but is not limited to, enzymes; prosthetic groups (e.g., biotin and flavine(s)); fluorescent materials; bioluminescent materials, such as luciferase; radioactive materials; positron emitting metals; and magnetic metal ions positron emitting metals; and magnetic metal ions.

Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, e.g., U.S. Pat. Nos. 5,021,236, 4,938,948, and 4,472,509). The imaging moieties used can be paramagnetic ions, radioactive isotopes, fluorochromes, NMR-detectable substances, MR hyperpolarized molecules, targeted ultrasound bubbles, and X-ray imaging agents.

An anti-IL-7Ra antibody or antigen-binding fragment described herein can be attached to a solid support. Such solid supports include, but are not limited to, glass, cellulose, polyacrylamide, nylon, polystyrene, polyvinyl chloride, or polypropylene. In some embodiments, an immobilized anti-IL-7Ra antibody or antigen-binding fragment is used in an immunoassay. In some embodiments, an immobilized anti-IL-7Ra antibody or antigen-binding fragment is used in purification of the target antigen (e.g., human IL-7Ra).

### 7.4 Polynucleotides and Vectors

Also provided herein are polynucleotides that encode a polypeptide (e.g., an anti-IL-7Ra antibody or antigen-binding fragment) described herein. The term “polynucleotide that encodes a polypeptide” encompasses a polynucleotide which includes only coding sequences for the polypeptide as well as a polynucleotide which includes additional coding and/or non-coding sequences. The polynucleotides of the disclosure can be in the form of RNA or in the form of DNA. DNA can be cDNA, genomic DNA, or synthetic DNA, and can be double-stranded or single-stranded. Single stranded DNA can be the coding strand or non-coding (anti-sense) strand. The polynucleotides of the disclosure can be mRNA.

Expressly contemplated herein are polynucleotides encode any anti-IL-7Ra antibody or antigen-binding fragment disclosed herein. For illustrative purposes, in some embodiments, the polynucleotides provided herein encode an anti-IL-7Ra antibody or antigen-binding fragment comprising (1) as defined by Kabat, (a) a light chain variable region (VL) comprising VL CDR1, VL CDR2, VL CDR3 having the amino acid sequences of SEQ ID NOs: 8, 9, and 10, respectively; or a variant thereof having up to about 5 amino acid substitutions, additions, and/or deletions in the VL CDRs; and/or (b) a heavy chain variable region (VH) comprising VH CDR1, VH CDR2, VH CDR3 having the amino acid sequences of SEQ ID NOs: 12, 14 and 15, respectively; or a variant thereof having up to about 5 amino acid substitutions, additions, and/or deletions in the VH CDRs; or (2) as defined by Chothia, (a) a VL comprising VL CDR1, VL CDR2, VL CDR3 having the amino acid sequences of SEQ ID NOs: 8, 9, and 10, respectively; or a variant thereof having up to about 5 amino acid substitutions, additions, and/or deletions in the VL CDRs; and/or (b) a VH comprising VH CDR1, VH CDR2, VH CDR3 having the amino acid sequences of SEQ ID NOs: 11, 13, and 15, respectively; or a variant thereof having up to about 5 amino acid substitutions, additions, and/or deletions in the VH CDRs.

In some embodiments, the polynucleotides provided herein encode an anti-IL-7Ra antibody or antigen-binding fragment comprising (a) a VL having at least 85%, at least 90%, at least 95%, at least 98%, or 100% sequence identity to the amino acid sequence of SEQ ID NO:18; and/or (b) a VH having at least 85%, at least 90%, at least 95%, at least 98%, or 100% sequence identity to the amino acid sequence of SEQ ID NO: 19. The polynucleotides provided herein can be in the form of DNA. The polynucleotides can be in the form of mRNA.

In some embodiments, the polynucleotides provided herein encode an anti-IL-7Ra antibody or antigen-binding fragment disclosed herein comprising a VL and a VH, wherein the VL comprises VL CDR1, CDR2 and CDR3 and the VH comprises VH CDR1, CDR2 and CDR3, and wherein the VL CDR1, VL CDR2, VL CDR3, VH CDR1, VH CDR2 and VH CDR3 have the amino acid sequences of (1) SEQ ID NOs: 8, 9, 10, 12, 14 and 15, respectively; or (2) SEQ ID NOs: 8, 9, 10, 11, 13, and 15, respectively; or a variant thereof having up to about 5 amino acid substitutions, additions, and/or deletions in the CDRs. The polynucleotides can be in the form of DNA. The polynucleotides can be in the form of mRNA.

In some embodiments, the polynucleotides provided herein encode an anti-IL-7Ra antibody or antigen-binding fragment disclosed herein comprising a VL and a VH, wherein the VL and VH have the amino acid sequences of (1) SEQ ID NOs: 20 and 26, respectively; (2) SEQ ID NOs: 20 and 27, respectively; (3) SEQ ID NOs: 20 and 28, respectively; (4) SEQ ID NOs: 20 and 29, respectively; (5) SEQ ID NOs: 20 and 30, respectively; (6) SEQ ID NOs: 21 and 26, respectively; (7) SEQ ID NOs: 21 and 27, respectively; (8) SEQ ID NOs: 21 and 28, respectively; (9) SEQ ID NOs: 21 and 29, respectively; (10) SEQ ID NOs: 21 and 30, respectively; (11) SEQ ID NOs: 22 and 26, respectively; (12) SEQ ID NOs: 22 and 27, respectively; (13) SEQ ID NOs: 22 and 28, respectively; (14) SEQ ID NOs: 22 and 29, respectively; (15) SEQ ID NOs: 22 and 30, respectively; (16) SEQ ID NOs: 23 and 26, respectively; (17) SEQ ID NOs: 23 and 27, respectively; (18) SEQ ID NOs: 23 and 28, respectively; (19) SEQ ID NOs: 23 and 29, respectively; or (20) SEQ ID NOs: 23 and 30, respectively. The polynucleotides can be in the form of DNA. The polynucleotides can be in the form of mRNA.

In some embodiments, the VL and VH are connected by a linker. The linker can be a flexible linker or a rigid linker. In some embodiments, the linker has the amino acid sequence of (GGGGS)n, n=1, 2, 3, 4, or 5 (SEQ ID NO:40). In some embodiments, the linker has the amino acid sequence of (EAAAK)n, n=1, 2, 3, 4, or 5 (SEQ ID NO:41). In some embodiments, the linker has the amino acid sequence of (PA)nPAP, n=0, 1, 2, 3, or 4 (SEQ ID NO:42).

The present disclosure also provides variants of the polynucleotides described herein, wherein the variants encode, for example, fragments, analogs, and/or derivatives of an anti-IL-7Ra antibody or antigen-binding fragment disclosed herein. In some embodiments, the present disclosure provides a polynucleotide having a nucleotide sequence at least about 80% identical, at least about 85% identical, at least about 90% identical, at least about 95% identical, at least about 96% identical, at least about 97% identical, at least about 98% identical, or at least about 99% identical to a polynucleotide sequence encoding an anti-IL-7Ra antibody or antigen-binding fragment described herein.

As used herein, the phrase “a polynucleotide having a nucleotide sequence at least about 95% identical to a polynucleotide sequence” means that the nucleotide sequence of the polynucleotide is identical to a reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (e.g., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, a polynucleotide comprises the coding sequence for a polypeptide (e.g., an antibody) fused in the same reading frame to a polynucleotide which aids in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In some embodiments, a polynucleotide comprises the coding sequence for a polypeptide (e.g., an antibody) fused in the same reading frame to a marker or tag sequence. For example, in some embodiments, a marker sequence is a hexa-histidine tag (HIS-tag) that allows for efficient purification of the polypeptide fused to the marker. In some embodiments, a marker sequence is a hemagglutinin (HA) tag derived from the influenza hemagglutinin protein when a mammalian host (e.g., COS-7 cells) is used. In some embodiments, the marker sequence is a FLAG™ tag. In some embodiments, a marker can be used in conjunction with other markers or tags.

In some embodiments, a polynucleotide is isolated. In some embodiments, a polynucleotide is substantially pure.

Vectors and cells comprising the polynucleotides described herein are also provided. In some embodiments, provided herein are vectors comprising a polynucleotide provided herein. The vectors can be expression vectors. In some embodiments, vectors provided herein comprise a polynucleotide encoding an anti-IL-7Ra antibody or antigen-binding fragment described herein. In some embodiments, vectors provided herein comprise a polynucleotide encoding a polypeptide that is part of an anti-IL-7Ra antibody or antigen-binding fragment described herein.

In some embodiments, provided herein are recombinant expression vectors, which can be used to amplify and express a polynucleotide encoding an anti-IL-7Ra antibody or antigen-binding fragment described herein. For example, a recombinant expression vector can be a replicable DNA construct that includes synthetic or cDNA-derived DNA fragments encoding a polypeptide chain of an anti-IL-7Ra antibody, operatively linked to suitable transcriptional and/or translational regulatory elements derived from mammalian, microbial, viral or insect genes. In some embodiments, a viral vector is used. DNA regions are “operatively linked” when they are functionally related to each other. For example, a promoter is operatively linked to a coding sequence if it controls the transcription of the sequence; or a ribosome binding site is operatively linked to a coding sequence if it is positioned so as to permit translation. In some embodiments, structural elements intended for use in certain expression systems include a leader sequence enabling extracellular secretion of translated protein by a host cell. In some embodiments, in situations where recombinant protein is expressed without a leader or transport sequence, a polypeptide can include an N-terminal methionine residue.

A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages. In some embodiments, an anti-IL-7Ra antibody or antigen-binding fragment described herein is expressed from one or more vectors.

Provided herein are suitable host cells comprising vectors described herein. In some embodiments, the host cells can be used for recombination expression of the anti-IL-7Ra antibodies described herein. The host cells can include prokaryotes, yeast cells, insect cells, or higher eukaryotic cells under the control of appropriate promoters. Appropriate cloning and expression vectors for use with bacterial, fungal, yeast, and mammalian cellular hosts, as well as methods of protein production, including antibody production are well-known in the art.

Examples of suitable mammalian host cells include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

The present disclosure also provides host cells comprising the polypeptides described herein, polynucleotides encoding polypeptides described herein, or vectors comprising such polynucleotides. In some embodiments, provided herein are host cells comprising a vector comprising a polynucleotide disclosed herein. In some embodiments, host cells provided herein comprise a vector comprising a polynucleotide encoding an anti-IL-7Ra antibody or antigen-binding fragment described herein. In some embodiments, host cells provided herein comprise a vector comprising a polynucleotide encoding a polypeptide that is part of an anti-IL-7Ra antibody or antigen-binding fragment described herein. In some embodiments, host cells provided herein comprise a polynucleotide encoding an anti-IL-7Ra antibody or antigen-binding fragment described herein. In some embodiments, the cells produce the anti-IL-7Ra antibodies or antigen-binding fragments described herein.

### 7.5 Methods of Manufacture

Provided herein are anti-IL-7Ra antibodies and antigen-binding fragments thereof that include but are not limited to monoclonal antibodies, polyclonal antibodies, synthetic antibodies, human antibodies, humanized antibodies, and antigen-binding fragments thereof. The anti-IL-7Ra antibodies or antigen-binding fragments described herein can be produced by any method known in the art, including chemical synthesis and recombinant expression techniques. The practice of the invention employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art.

In some embodiments, monoclonal antibodies are made using recombinant DNA techniques as known to one skilled in the art. Polynucleotides of the antibodies or antigen-binding fragments provided herein can be prepared, manipulated, and/or expressed using any of the well-established techniques known and available in the art. In some embodiments, polynucleotides of the antibodies or antigen-binding fragments provided herein can be prepared recombinantly. Many vectors can be used. Exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells.

In some embodiments, a recombinant expression vector is used to express a polynucleotide encoding a polypeptide described herein. For example, a recombinant expression vector can be a replicable DNA construct that includes synthetic or cDNA-derived DNA fragments encoding a polypeptide operatively linked to suitable transcriptional and/or translational regulatory elements derived from mammalian, microbial, viral or insect genes. In some embodiments, coding sequences of polypeptides disclosed herein can be ligated into such expression vectors for their expression in mammalian cells. In some embodiments, a viral vector is used. DNA regions are “operatively linked” when they are functionally related to each other. For example, a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In some embodiments, in situations where recombinant protein is expressed without a leader or transport sequence, a polypeptide can include an N-terminal methionine residue.

A wide variety of expression host/vector combinations can be employed. Suitable host cells for expression include prokaryotes, yeast cells, insect cells, or higher eukaryotic cells under the control of appropriate promoters. Appropriate cloning and expression vectors for use with bacterial, fungal, yeast, and mammalian cellular hosts, as well as methods of protein production, including antibody production are well-known in the art. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Useful expression vectors for eukaryotic hosts include, for example, vectors comprising expression control sequences from SV40, bovine papilloma virus, adenovirus, and cytomegalovirus. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

To make anti-IL-7Ra antibodies and antigen-binding fragments that are afucosylated, host cells that (1) overexpress N-acetylglucosaminyltransferase III (GnTIII), (2) lack α-1,6-fucosyltransferase (FUT8), or (3) have a low fucose content, or any combination of (1)-(3) can be used. In some embodiments, used herein are host cells that overexpresses N-acetylglucosaminyltransferase III (GnTIII). In some embodiments, used herein are host cells that lack α-1,6-fucosyltransferase (FUT8). In some embodiments, used herein are host cells having a low fucose content. In some embodiments, CHO host cells are used.

Peptides can also be synthesized, in whole or in part, using chemical methods (see, e.g., Caruthers (1980). Nucleic Acids Res. Symp. Ser. 215; Horn (1980); and Banga, A. K., THERAPEUTIC PEPTIDES AND PROTEINS, FORMULATION, PROCESSING AND DELIVERY SYSTEMS (1995) Technomic Publishing Co., Lancaster, PA). Peptide synthesis can be performed using various solid phase techniques (see, e.g., Roberge, Science 269:202 (1995); Merrifield, Methods. Enzymol. 289:3 (1997)) and automated synthesis may be achieved, e.g., using the ABI 431A Peptide Synthesizer (Perkin Elmer) in accordance with the manufacturer's instructions. Peptides can also be synthesized using combinatorial methodologies. Synthetic residues and polypeptides can be synthesized using a variety of procedures and methodologies known in the art (see, e.g., ORGANIC SYNTHESES COLLECTIVE VOLUMES, Gilman, et al. (Eds) John Wiley & Sons, Inc., NY). Modified peptides can be produced by chemical modification methods (see, for example, Belousov, Nucleic Acids Res. 25:3440 (1997); Frenkel, Free Radic. Biol. Med. 19:373 (1995); and Blommers, Biochemistry 33:7886 (1994)). Peptide sequence variations, derivatives, substitutions and modifications can also be made using methods such as oligonucleotide-mediated (site-directed) mutagenesis, alanine scanning, and PCR based mutagenesis. Site-directed mutagenesis (Carter et al., Nucl. Acids Res., 13:4331 (1986); Zoller et al., Nucl. Acids Res. 10:6487 (1987)), cassette mutagenesis (Wells et al., Gene 34:315 (1985)), restriction selection mutagenesis (Wells et al., Philos. Trans. R. Soc. London SerA 317:415 (1986)) and other techniques can be performed on cloned DNA to produce invention peptide sequences, variants, fusions and chimeras, and variations, derivatives, substitutions and modifications thereof.

For in vivo use of antibodies in humans, it may be preferable to use human or humanized antibodies. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. A human antibody can also be an antibody wherein the heavy and light chains are encoded by a nucleotide sequence derived from one or more sources of human DNA. In some embodiments, an anti-IL-7Ra antibody or antigen-binding fragment is a human antibody or antigen-binding fragment. Human antibodies can be prepared using various techniques known in the art.

Alternatively, in some embodiments, a non-human antibody is humanized, where specific sequences or regions of the antibody are modified to increase similarity to an antibody naturally produced in a human. In some embodiments, the antigen binding domain portion is humanized. Various methods for generating humanized antibodies are known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28 (4/5): 489-498; Studnicka et al., 1994, Protein Engineering, 7 (6): 805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 93/17105, Tan et al., J. Immunol., 169:1119-25 (2002), Caldas et al., Protein Eng., 13 (5): 353-60 (2000), Morea et al., Methods, 20 (3): 267-79 (2000), Baca et al., J. Biol. Chem., 272 (16): 10678-84 (1997), Roguska et al., Protein Eng., 9 (10): 895-904 (1996), Couto et al., Cancer Res., 55 (23 Supp): 5973s-5977s (1995), Couto et al., Cancer Res., 55 (8): 1717-22 (1995), Sandhu J S, Gene, 150 (2): 409-10 (1994), and Pedersen et al., J. Mol. Biol., 235 (3): 959-73 (1994), each of which is incorporated herein in its entirety by reference. Often, framework residues in the framework regions can be substituted with the corresponding residue from the CDR donor antibody to alter, preferably improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized chimeric antibodies, substantially less than an intact human variable domain has been substituted by the corresponding sequence from a nonhuman species. In practice, humanized antibodies are typically human antibodies in which some CDR residues and possibly some FR residues are substituted by residues from analogous sites in rodent antibodies. Humanization of antibodies can also be achieved by veneering or resurfacing (EP 592,106; EP 519,596; Padlan, 1991, Molecular Immunology, 28 (4/5): 489-498; Studnicka et al., Protein Engineering, 7 (6): 805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993), the contents of which are incorporated herein by reference herein in their entirety).

Antibodies can be humanized with retention of high affinity for the target antigen and other favorable biological properties. For example, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

A humanized antibody retains a similar antigenic specificity as the original antibody, for example, the ability to bind human IL-7Ra antigen. However, using certain methods of humanization, the affinity and/or specificity of binding of the antibody for a particular antigen can be increased using methods of “directed evolution,” as described by Wu et al., J. Mol. Biol., 294:151 (1999), the contents of which are incorporated herein by reference herein in their entirety.

A variety of methods are known in the art to purify anti-IL-7Ra antibodies, such as affinity chromatography. Eluted IgG can be checked by gel electrophoresis and high-performance liquid chromatography to ensure purity. The buffer solution can be exchanged, and the concentration can be determined. The monoclonal antibodies can be aliquoted and stored.

Methods for analyzing binding affinity, cross-reactivity, and binding kinetics of various anti-IL-7Ra antibodies include standard assays known in the art, for example, Western Blot, ELISA, and flow cytometry. Further methods available in the art include biolayer interferometry (BLI) using, for example, Gator system (Probe Life) or the Octet-96 system (Sartorius AG), or BIACORE™ surface plasmon resonance (SPR) analysis using a BIACORE™ 2000 SPR instrument (Biacore AB, Uppsala, Sweden).

### 7.6 Pharmaceutical Compositions

Provided herein are also pharmaceutical compositions comprising the anti-IL-7Ra antibodies or antigen-binding fragments disclosed herein. In some embodiments, the pharmaceutical composition comprises a therapeutically effective amount of the anti-IL-7Ra antibodies or antigen-binding fragments disclosed herein and a pharmaceutically acceptable carrier. In some embodiments, the pharmaceutical compositions are useful in suppressing autoimmunity or inflammation related to IL-7 and/or TSLP. In some embodiments, the pharmaceutical compositions are useful in treating a disease or disorder associated with IL-7 and/or TSLP.

In some embodiments, the pharmaceutical compositions provided herein comprise anti-IL-7Ra antibodies or antigen-binding fragments provided herein. The anti-IL-7Ra antibodies or antigen-binding fragments can be present at various concentrations. In some embodiments, the pharmaceutical compositions provided herein comprise soluble anti-IL-7Ra antibodies or antigen-binding fragments provided herein at 1-1000 mg/mL. Dosages can be readily adjusted by those skilled in the art; for example, a decrease in purity may require an increase in dosage.

Provided herein are also kits for preparation of pharmaceutical compositions having the anti-IL-7Ra antibodies or antigen-binding fragments disclosed herein. In some embodiments, the kit comprises the anti-IL-7Ra antibodies or antigen-binding fragments disclosed herein and a pharmaceutically acceptable carrier in one or more containers. In another embodiment, the kits can comprise anti-IL-7Ra antibodies or antigen-binding fragments disclosed herein for administration to a subject. In specific embodiments, the kits comprise instructions regarding the preparation and/or administration of the anti-IL-7Ra antibodies or antigen-binding fragments.

In some embodiments, provided herein is a pharmaceutical composition comprising anti-IL-7Ra antibodies or antigen-binding fragments or cells provided herein wherein the composition is suitable for local administration. In some embodiments, provided herein is a pharmaceutical composition comprising anti-IL-7Ra antibodies or antigen-binding fragments or cells provided herein wherein the composition is suitable for systemic administration.

Pharmaceutically acceptable carriers that can be used in compositions provided herein include any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active ingredient (i.e., anti-IL-7Ra antibodies or antigen-binding fragments) can be coated in a material to protect the active ingredient from the action of acids and other natural conditions that can inactivate the active ingredient.

Provided herein are also pharmaceutical compositions or formulations that improve the stability of the anti-IL-7Ra antibodies or antigen-binding fragments to allow for their long-term storage. In some embodiments, the pharmaceutical composition or formulation disclosed herein comprises: (a) anti-IL-7Ra antibodies or antigen-binding fragments disclosed herein; (b) a buffering agent; (c) a stabilizing agent; (d) a salt; (e) a bulking agent; and/or (f) a surfactant. In some embodiments, the pharmaceutical composition or formulation is stable for at least 1 month, at least 2 months, at least 3 months, at least 6 months, at least 1 year, at least 2 years, at least 3 years, at least 5 years or more. In some embodiments, the pharmaceutical composition or formulation is stable when stored at 4° C., 25° C., or 40° C.

Buffering agents useful in the pharmaceutical compositions or formulations disclosed herein can be a weak acid or base used to maintain the acidity (pH) of a solution near a chosen value after the addition of another acid or base. Suitable buffering agents can maximize the stability of the pharmaceutical formulations by maintaining pH control of the formulation. Suitable buffering agents can also ensure physiological compatibility or optimize solubility. Rheology, viscosity and other properties can also depend on the pH of the formulation. Common buffering agents include, but are not limited to, histidine, citrate, succinate, acetate and phosphate. In some embodiments, a buffering agent comprises histidine (e.g., L-histidine) with isotonicity agents and potentially pH adjustment with an acid or a base known in the art. In certain embodiments, the buffering agent is L-histidine. In certain embodiments, the pH of the formulation is maintained between about 2 and about 10, or between about 4 and about 8.

Stabilizing agents are added to a pharmaceutical product to stabilize that product. Such agents can stabilize proteins in different ways. Common stabilizing agents include, but are not limited to, amino acids such as glycine, alanine, lysine, arginine, or threonine, carbohydrates such as glucose, sucrose, trehalose, rafftnose, or maltose, polyols such as glycerol, mannitol, sorbitol, cyclodextrins or dextran of any kind and molecular weight, or PEG. In some embodiments, the stabilizing agent is chosen to maximize the stability of FIX polypeptide in lyophilized preparations. In certain embodiments, the stabilizing agent is sucrose and/or arginine.

Bulking agents can be added to a pharmaceutical composition or formulation to add volume and mass to the product, thereby facilitating precise metering and handling thereof. Common bulking agents include, but are not limited to, lactose, sucrose, glucose, mannitol, sorbitol, calcium carbonate, or magnesium stearate.

Surfactants are amphipathic substances with lyophilic and lyophobic groups. A surfactant can be anionic, cationic, zwitterionic, or nonionic. Examples of nonionic surfactants include, but are not limited to, alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, or dodecyl dimethylamine oxide. In some embodiments, the surfactant is polysorbate 20 or polysorbate 80.

The pharmaceutical compositions disclosed herein can further comprise one or more of a buffer system, a preservative, a tonicity agent, a chelating agent, a stabilizer and/or a surfactant, as well as various combinations thereof. The use of preservatives, isotonic agents, chelating agents, stabilizers and surfactants in pharmaceutical compositions is well-known to the skilled person. Reference may be made to Remington: The Science and Practice of Pharmacy, 19^{th} edition, 1995.

In some embodiments, the pharmaceutical composition is an aqueous formulation. Such a formulation is typically a solution or a suspension, but can also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In some embodiments, the pharmaceutical compositions disclosed herein are freeze-dried, to which the physician or the patient adds solvents and/or diluents prior to use.

Pharmaceutical compositions disclosed herein can also include a pharmaceutically acceptable antioxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that can be employed in the pharmaceutical compositions or formulations described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions described herein is contemplated. A pharmaceutical composition or formulation can comprise a preservative or can be devoid of a preservative. Supplementary active compounds can be incorporated into the compositions.

Pharmaceutical compositions or formulations typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, the compositions can include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

The amount of active ingredient which can be combined with a carrier material in the pharmaceutical compositions or formulations disclosed herein can vary. In some embodiments, the amount of active ingredient which can be combined with a carrier material is the amount that produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

The pharmaceutical compositions disclosed herein can be prepared with carriers that protect the active ingredient against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and poly lactic acid. Many methods for the preparation of such formulations are patented or generally known to those skilled in the art. See. e.g., Sustained and Controlled Release Drug Delivery Systems, J. R. Robinson, ed., Marcel Dekker, Inc., New York, 1978.

### 7.7 Methods and Uses

The anti-IL-7Ra antibodies or antigen-binding fragments, compositions and methods described herein have numerous in vitro and in vivo utilities. The present disclosure also provides methods of uses of the anti-IL-7Ra antibodies or antigen-binding fragments, polynucleotides encoding such anti-IL-7Ra antibodies or antigen-binding fragments, vectors comprising such polynucleotides, or pharmaceutical compositions having such antibodies or antigen-binding fragments disclosed herein in reducing IL-7 signaling and/or TSLP signaling, or in treating a disease or disorder associated with IL-7 signaling and/or TSLP signaling, such as autoimmune and inflammatory disease.

In some embodiments, provided herein are methods of reducing IL-7 signaling in a subject in need thereof comprising administering to the subject a therapeutically effective amount of the anti-IL-7Ra antibodies or antigen-binding fragments disclosed herein. In some embodiments, provided herein are uses of the anti-IL-7Ra antibodies or antigen-binding fragments disclosed herein for reducing IL-7 signaling. In some embodiments, provided herein are uses of the anti-IL-7Ra antibodies or antigen-binding fragments provided herein for the preparation of a medicament for reducing IL-7 signaling. In some embodiments, provided herein are methods of reducing IL-7 signaling in a subject in need thereof comprising administering to the subject a therapeutically effective amount of the pharmaceutical compositions disclosed herein. In some embodiments, provided herein are uses of the pharmaceutical compositions disclosed herein for reducing IL-7 signaling. In some embodiments, provided herein are uses of the pharmaceutical compositions provided herein for the preparation of a medicament for reducing IL-7 signaling. In some embodiments, the methods provided herein block IL-7 binding to IL-7Ra. In some embodiments, the methods provided herein block IL-7-induced STAT-5 phosphorylation. In some embodiments, the methods provided herein block IL-7-induced T cell proliferation.

In some embodiments, provided herein are methods of reducing TSLP signaling in a subject in need thereof comprising administering to the subject a therapeutically effective amount of the anti-IL-7Ra antibodies or antigen-binding fragments disclosed herein. In some embodiments, provided herein are uses of the anti-IL-7Ra antibodies or antigen-binding fragments disclosed herein for reducing TSLP signaling. In some embodiments, provided herein are uses of the anti-IL-7Ra antibodies or antigen-binding fragments provided herein for the preparation of a medicament for reducing TSLP signaling. In some embodiments, provided herein are methods of reducing TSLP signaling in a subject in need thereof comprising administering to the subject a therapeutically effective amount of the pharmaceutical compositions disclosed herein. In some embodiments, provided herein are uses of the pharmaceutical compositions disclosed herein for reducing TSLP signaling. In some embodiments, provided herein are uses of the pharmaceutical compositions provided herein for the preparation of a medicament for reducing TSLP signaling. In some embodiments, the anti-IL-7Ra antibodies or antigen-binding fragments disclosed herein block TSLP signaling in a subject in need thereof. In some embodiments, the methods provided herein block TSLP-induced TARC secretion.

In some embodiments, provided herein are methods of reducing autoimmunity or inflammation in a subject in need thereof comprising administering to the subject a therapeutically effective amount of the anti-IL-7Ra antibodies or antigen-binding fragments disclosed herein. In some embodiments, provided herein are uses of the anti-IL-7Ra antibodies or antigen-binding fragments disclosed herein for reducing autoimmunity or inflammation. In some embodiments, provided herein are uses of the anti-IL-7Ra antibodies or antigen-binding fragments provided herein for the preparation of a medicament for reducing autoimmunity or inflammation. In some embodiments, provided herein are methods of reducing autoimmunity or inflammation in a subject in need thereof comprising administering to the subject a therapeutically effective amount of the pharmaceutical compositions disclosed herein. In some embodiments, provided herein are uses of the pharmaceutical compositions disclosed herein for reducing autoimmunity or inflammation. In some embodiments, provided herein are uses of the pharmaceutical compositions provided herein for the preparation of a medicament for reducing autoimmunity or inflammation.

Dysregulated or excessive activation of IL-7 and/or TSLP signaling pathway leads to unwanted activation of the immune system, which has been implicated in the pathogenesis of a wide variety of diseases, such as autoimmune and inflammatory diseases. In some embodiments, provided herein are methods of treating an autoimmune or inflammatory disease in a subject in need thereof comprising administering to the subject a therapeutically effective amount of the anti-IL-7Ra antibodies or antigen-binding fragments disclosed herein. In some embodiments, provided herein are uses of the anti-IL-7Ra antibodies or antigen-binding fragments disclosed herein for treating an autoimmune or inflammatory disease. In some embodiments, provided herein are uses of the anti-IL-7Ra antibodies or antigen-binding fragments provided herein for the preparation of a medicament for treating an autoimmune or inflammatory disease. In some embodiments, provided herein are methods of treating an autoimmune or inflammatory disease in a subject in need thereof comprising administering to the subject a therapeutically effective amount of the pharmaceutical compositions disclosed herein. In some embodiments, provided herein are uses of the pharmaceutical compositions disclosed herein for treating an autoimmune or inflammatory disease. In some embodiments, provided herein are uses of the pharmaceutical compositions provided herein for the preparation of a medicament for treating an autoimmune or inflammatory disease. The disease to be treated with methods disclosed herein can be an autoimmune or inflammatory disease associated with IL-7, TSLP, and/or IL-7Ra.

In some embodiments, the disease to be treated with methods disclosed herein is an IL-7-associated autoimmune or inflammatory disease. In some embodiments, the disease to be treated with methods disclosed herein is a TSLP-associated autoimmune or inflammatory disease. In some embodiments, the disease to be treated with methods disclosed herein is an IL-7Ra-associated autoimmune or inflammatory disease. In some embodiments, the disease associated with IL-7 and/or TSLP is ulcerative colitis (UC). In some embodiments, the disease or disorder that can be treated with the anti-IL-7Ra antibodies or antigen-binding fragments, or pharmaceutical compositions provided herein is UC.

The subject to be treated by methods disclosed herein is typically a human. The subject can also be a mammal, such as a mouse, rat or primate (e.g., a marmoset or monkey). The subject can be a non-human animal. The IL-7 binding proteins may also have veterinary use. The subject to be treated can be a farm animal for example, a cow or bull, sheep, pig, ox, goat or horse or may be a domestic animal such as a dog or cat. The animal can be any age, or a mature adult animal. In some embodiments, treatment can be therapeutic, prophylactic or preventative. The subject can be one who is in need thereof. Those in need of treatment may include individuals already suffering from a medical disease in addition to those who may develop the disease in the future. In some embodiments, the subject to be treated with methods disclosed herein has excess IL-7 signaling and/or TSLP signaling. In some embodiments, the subject to be treated with methods disclosed herein has excess autoimmunity or inflammation. In some embodiments, the subject has been diagnosed with an autoimmune or inflammatory disease associated with IL-7 signaling and/or TSLP-signaling. In some embodiments, the subject is at risk of developing an autoimmune or inflammatory disease associated with IL-7 signaling and/or TSLP-signaling. In some embodiments, the subject has been diagnosed with UC. In some embodiments, the subject is at risk of developing UC.

In some embodiments, methods provided herein can promote a beneficial therapeutic response with respect to an autoimmune or inflammatory response. In some embodiments, methods provided herein result in an improvement of symptoms associated with the disease.

Actual dosage levels of the active ingredients (i.e., the anti-IL-7Ra antibodies or antigen-binding fragments) in the pharmaceutical compositions described herein can be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions described herein, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

The anti-IL-7Ra antibodies or antigen-binding fragments can be administered as a sustained release formulation, in which case less frequent administration is required. Dosage and frequency vary depending on the half-life of the anti-IL-7Ra antibodies or antigen-binding fragments in the patient. In therapeutic applications, a relatively high dosage at relatively short intervals is sometimes required until progression of the disease is reduced or terminated, and until the patient shows partial or complete amelioration of symptoms of disease.

The anti-IL-7Ra antibodies or antigen-binding fragments or pharmaceutical compositions provided herein can be administered to a subject by any methods known in the art, including, but not limited to, pleural administration, intravenous administration, subcutaneous administration, intranodal administration, intramuscular administration, intradermal administration, intrathecal administration, intrapleural administration, intraperitoneal administration, intracranial administration, spinal or other parenteral routes of administration, for example by injection or infusion, or direct administration to the thymus. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. In some embodiments, subcutaneous administration is adopted. In some embodiments, intravenous administration is adopted. In some embodiments, oral administration is adopted. In one embodiment, the antibodies or antigen-binding fragments provided herein can be delivered locally. In another embodiment, the antibodies or antigen-binding fragments provided herein can be administered systemically.

In the methods disclosed herein, a therapeutically effective amount of the anti-IL-7Ra antibodies or antigen-binding fragments or pharmaceutical compositions disclosed herein is administered to a subject that can benefit from blockade of IL-7 signaling or TSLP signaling The subject can have unwanted, unregulated, or excessive activation of IL-7 and/or TSLP signaling pathway. The subject can be a mammal. In some embodiments, the subject is a human.

Anti-IL-7Ra antibodies or antigen-binding fragments or pharmaceutical compositions provided herein can be administered with medical devices known in the art. For example, in some embodiments, a needleless hypodermic injection device can be used, such as the devices disclosed in U.S. Pat. Nos. 5,399,163; 5,383,851; 5,312,335; 5,064,413; 4,941,880; 4,790,824; or 4,596,556. Examples of well-known implants and modules for use described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In some embodiments, the anti-IL-7Ra antibodies or antigen-binding fragments or pharmaceutical compositions provided herein can be administered with an additional therapy. The additional therapy can be administered prior to, concurrently with, or subsequent to administration of the anti-IL-7Ra antibodies or antigen-binding fragments, cells, or pharmaceutical compositions described herein. Additional treatments for UC include, for example, anti-inflammatory medication, immunosuppressant, corticosteroid, etc. Combined administration can include co-administration, either in a single pharmaceutical formulation or using separate formulations, or consecutive administration in either order but generally within a time period such that all active agents can exert their biological activities simultaneously. A person skilled in the art can readily determine appropriate regimens for administering a pharmaceutical composition described herein and an additional therapy in combination, including the timing and dosing of an additional agent to be used in a combination therapy, based on the needs of the subject being treated.

The antibodies or antigen-binding fragments provided herein can also be used in detection of IL-7Ra. Also encompassed are methods for detecting the presence of human IL-7Ra antigen in a sample, or measuring the amount of human IL-7Ra antigen, comprising contacting the sample, and a control sample, with a monoclonal antibody, e.g., a humanized monoclonal antibody, or an antigen-binding fragment thereof, which specifically binds to human IL-7Ra, under conditions that allow for formation of a complex between the antibody or antigen-binding fragment and human IL-7Ra. The formation of a complex is then detected, wherein a difference complex formation between the sample compared to the control sample is indicative the presence of human IL-7Ra antigen in the sample. Moreover, the anti-IL-7Ra antibodies or antigen-binding fragments described herein can be used to purify human IL-7Ra via immunoaffinity purification. In some embodiments, the anti-IL-7Ra antibodies or antigen-binding fragments described herein are used for detecting IL-7Ra-expressing cells. In some embodiments, the anti-IL-7Ra antibodies or antigen-binding fragments described herein are used for quantifying IL-7Ra antigen, or IL-7Ra-expressing cells.

In some embodiments, an IL-7 binding protein disclosed herein is used in a method of diagnosis or prognosis. In some embodiments, diagnosis includes determining whether a subject has a disease or condition and/or determining the severity of the disease or condition. In some embodiments, prognosis includes predicting whether or not a subject will develop a disease or condition, whether or not they will need treatment, the type of treatment the individual will need, whether or not they will respond to a treatment, whether or not and/or when they will suffer a disease episode, recurrence or relapse, and the severity or duration of a symptom or a disease episode, recurrence or relapse. In some embodiments, a method of diagnosis or prognosis may include selecting or recommending a suitable treatment for the individual, for example, based on the diagnosis or prognosis. In some embodiments, a selected or recommended treatment or combination of treatments may then be administered to the subject.

In some embodiments, the anti-IL-7Ra antibodies or antigen-binding fragments disclosed herein can be used in a method of diagnosis or prognosis. In some embodiments, diagnosis includes determining whether a subject has a disease or condition and/or determining the severity of the disease or condition. In some embodiments, prognosis includes predicting whether or not a subject will develop a disease or condition, whether or not they will need treatment, the type of treatment the individual will need, whether or not they will respond to a treatment, whether or not and/or when they will suffer a disease episode, recurrence or relapse, and the severity or duration of a symptom or a disease episode, recurrence or relapse. In some embodiments, a method of diagnosis or prognosis may include selecting or recommending a suitable treatment for the individual, for example, based on the diagnosis or prognosis. In some embodiments, a selected or recommended treatment or combination of treatments may then be administered to the subject.

In some embodiments, a method for predicting the responsiveness of a subject to a treatment can be carried out before administration a therapy. The prediction can then be taken into account when selecting or recommending a suitable treatment for the individual. Alternatively, a method of predicting responsiveness to a treatment can be carried out after treatment with a therapy and used to monitor and predict the subject's response to treatment.

### 7.8 Exemplified Embodiments

Embodiment 1: An antibody or antigen-binding fragment thereof that specifically binds human IL-7Ra, comprising: (1) as defined by Kabat, (a) a light chain variable region (VL) comprising VL CDR1, VL CDR2, VL CDR3 having the amino acid sequences of SEQ ID NOs: 8, 9, and 10, respectively; or a variant thereof having up to about 5 amino acid substitutions, additions, and/or deletions in the VL CDRs; and/or (b) a heavy chain variable region (VH) comprising VH CDR1, VH CDR2, VH CDR3 having the amino acid sequences of SEQ ID NOs: 12, 14 and 15, respectively; or a variant thereof having up to about 5 amino acid substitutions, additions, and/or deletions in the VH CDRs; or (2) as defined by Chothia, (a) a VL comprising VL CDR1, VL CDR2, VL CDR3 having the amino acid sequences of SEQ ID NOs: 8, 9, and 10, respectively; or a variant thereof having up to about 5 amino acid substitutions, additions, and/or deletions in the VL CDRs; and/or (b) a VH comprising VH CDR1, VH CDR2, VH CDR3 having the amino acid sequences of SEQ ID NOs: 11, 13, and 15, respectively; or a variant thereof having up to about 5 amino acid substitutions, additions, and/or deletions in the VH CDRs.

Embodiment 2: The antibody or antigen-binding fragment of claim 1, comprising VL CDR1, VL CDR2, VL CDR3, VH CDR1, VH CDR2 and VH CDR3 having the amino acid sequences of SEQ ID NOs: 8, 9, 10, 12, 14 and 15, respectively, as defined by Kabat.

Embodiment 3: The antibody or antigen-binding fragment of claim 1, comprising VL CDR1, VL CDR2, VL CDR3, VH CDR1, VH CDR2 and VH CDR3 having the amino acid sequences of SEQ ID NOs: 8, 9, 10, 11, 13, and 15, respectively, as defined by Chothia.

Embodiment 4: An antibody or antigen-binding fragment thereof that specifically binds human IL-7Ra, comprising: (a) a VL having at least 85%, at least 90%, at least 95%, at least 98%, or 100% sequence identity to the amino acid sequence of SEQ ID NO: 18; and/or (b) a VH having at least 85%, at least 90%, at least 95%, at least 98%, or 100% sequence identity to the amino acid sequence of SEQ ID NO:19.

Embodiment 5: The antibody or antigen-binding fragment of Embodiment 4 comprising a VL and a VH having the amino acid sequences of SEQ ID NOs: 18 and 19, respectively.

Embodiment 6: An antibody or antigen-binding fragment thereof that specifically binds human IL-7Ra, comprising (a) a VL comprising VL CDR1, VL CDR2, and VL CDR3 from a VL having the amino acid sequence of SEQ ID NO:18; and/or (b) a VH comprising VH CDR1, VH CDR2, and VH CDR3 from a VH having the amino acid sequence of SEQ ID NO: 19.

Embodiment 7: The antibody or antigen-binding fragment of any one of Embodiments 1 to 6 that is a chimeric antibody or antigen-binding fragment, a humanized antibody or antigen-binding fragment, or a human antibody or antigen-binding fragment.

Embodiment 8: The antibody or antigen-binding fragment of Embodiment 7 that is a humanized antibody or antigen-binding fragment.

Embodiment 9: The antibody or antigen-binding fragment of Embodiment 8, comprising:
(a) a VL having at least 85%, at least 90%, at least 95%, at least 98%, or 100% sequence identity to an amino acid sequence selected from the group consisting of SEQ ID NOs: 20-23; and/or
(b) a VH having at least 85%, at least 90%, at least 95%, at least 98%, or 100% sequence identity to an amino acid sequence selected from the group consisting of SEQ ID NOs: 26-30.

Embodiment 10: The antibody or antigen-binding fragment of Embodiment 9 comprising a VL and a VH having the amino acid sequences of (1) SEQ ID NOs: 20 and 26, respectively; (2) SEQ ID NOs: 20 and 27, respectively; (3) SEQ ID NOs: 20 and 28, respectively; (4) SEQ ID NOs: 20 and 29, respectively; (5) SEQ ID NOs: 20 and 30, respectively; (6) SEQ ID NOs: 21 and 26, respectively; (7) SEQ ID NOs: 21 and 27, respectively; (8) SEQ ID NOs: 21 and 28, respectively; (9) SEQ ID NOs: 21 and 29, respectively; (10) SEQ ID NOs: 21 and 30, respectively; (11) SEQ ID NOs: 22 and 26, respectively; (12) SEQ ID NOs: 22 and 27, respectively; (13) SEQ ID NOs: 22 and 28, respectively; (14) SEQ ID NOs: 22 and 29, respectively; (15) SEQ ID NOs: 22 and 30, respectively; (16) SEQ ID NOs: 23 and 26, respectively; (17) SEQ ID NOs: 23 and 27, respectively; (18) SEQ ID NOs: 23 and 28, respectively; (19) SEQ ID NOs: 23 and 29, respectively; or (20) SEQ ID NOs: 23 and 30, respectively.

Embodiment 11: The antibody or antigen-binding fragment of any one of Embodiments 1 to 10 that is selected from the group consisting of a Fab, a Fab′, a F(ab′) 2, a Fv, a scFv, a (scFv) 2, a single domain antibody (sdAb), and a heavy chain antibody (HCAb).

Embodiment 12: The antibody or antigen-binding fragment of any one of Embodiments 1 to 10 that is an IgG1 antibody, an IgG2 antibody, an IgG3 antibody, or an IgG4 antibody.

Embodiment 13: The antibody of Embodiment 12 comprising a light chain constant region (CL) having at least 85% sequence identity to kappa CL (CK; SEQ ID NO:33).

Embodiment 14: The antibody of Embodiment 12 comprising a light chain constant region (CL) having at least 85% sequence identity to lambda CL (CA; SEQ ID NO:34).

Embodiment 15: The antibody of Embodiment 12 comprising a heavy chain constant region (CH) having at least 85% sequence identity to an amino acid sequence selected from the group consisting of SEQ ID NOs: 35-38.

Embodiment 16: The antibody of Embodiment 12 that is an IgG4 antibody.

Embodiment 17: The antibody of Embodiment 16, wherein the heavy chain constant region (CH) of the IgG4 antibody comprises a wildtype IgG4 CH, or comprises at least one amino acid mutation.

Embodiment 18: The antibody of Embodiment 17, wherein the CH region of the IgG4 antibody has S228P substitution (SEQ ID NO:39).

Embodiment 19: The antibody of any one of Embodiments 16 to 18, wherein the Fc region of the antibody is afucosylated.

Embodiment 20: An antibody or antigen-binding fragment thereof that competes with the antibody or antigen-binding fragment of any one of Embodiments 1 to 19 for binding to human IL-7Ra.

Embodiment 21: The antibody or antigen-binding fragment of any one of Embodiments 1 to 20 that is a bispecific antibody or a multispecific antibody.

Embodiment 22: The antibody or antigen-binding fragment of any one of Embodiments 1 to 21 that is a monoclonal antibody or antigen-binding fragment.

Embodiment 23: The antibody or antigen-binding fragment of any one of Embodiments 1 to 22, wherein the antibody or antigen-binding fragment: (1) binds to human IL-7Ra with high affinity; (2) blocks IL-7 binding to IL-7Ra; (3) blocks IL-7-induced STAT5 phosphorylation; (4) blocks IL-7-induced T cell proliferation; (5) blocks TSLP induced TARC secretion; or (6) ameliorates colitis; or any combination of (1)-(6).

Embodiment 24: An antibody or antigen-binding fragment thereof that specifically binds human IL-7Ra, wherein the antibody or antigen-binding fragment: (1) binds to human IL-7Ra with high affinity; (2) blocks IL-7 binding to IL-7Ra; (3) blocks IL-7-induced STAT5 phosphorylation; (4) blocks IL-7-induced T cell proliferation; (5) blocks TSLP induced TARC secretion; or (6) ameliorates colitis; or any combination of (1)-(6).

Embodiment 25: A polynucleotide encoding a polypeptide of the antibody or antigen-binding fragment of any one of Embodiments 1 to 24.

Embodiment 26: A vector comprising the polynucleotide of Embodiment 25.

Embodiment 27: A host cell comprising the polynucleotide of Embodiment 25, or the vector of Embodiment 26.

Embodiment 28: The host cell of Embodiment 27, that (1) overexpresses N-acetylglucosaminyltransferase III (GnTIII), (2) lacks α-1,6-fucosyltransferase (FUT8), or (3) has a low fucose content, or any combination of (1)-(3).

Embodiment 29: A method of making an antibody or antigen-binding fragment thereof that specifically binds human IL-7Ra, comprising culturing the cell of Embodiment 27 or 28 under conditions that allow expression of the antibody or antigen-binding fragment.

Embodiment 30: The method of Embodiment 29 that comprises isolating the antibody or antigen-binding fragment from the culture.

Embodiment 31: A pharmaceutical composition comprising a therapeutically effective amount of the antibody or antigen-binding fragment of any one of Embodiments 1 to 24, and a pharmaceutically acceptable carrier.

Embodiment 32: A method of reducing IL-7 signaling and/or TSLP signaling in a subject in need thereof, comprising administering to the subject a therapeutically effective amount of the antibody or antigen-binding fragment of any one of Embodiments 1 to 24.

Embodiment 33: A method of reducing autoimmunity or inflammation in a subject in need thereof, comprising administering to the subject an effective amount of the antibody or antigen-binding fragment of any one of Embodiments 1 to 24.

Embodiment 34: The method of any one of Embodiments 32 to 33, wherein the subject has an autoimmune or inflammatory disease.

Embodiment 35: A method of treating an autoimmune or inflammatory disease associated with IL-7 and/or TSLP in a subject in need thereof, comprising administering to the subject a therapeutically effective amount of the antibody or antigen-binding fragment of any one of Embodiments 1 to 24.

Embodiment 36: The method of Embodiment 34 or 35, wherein the autoimmune or inflammatory disease is ulcerative colitis (UC).

Embodiment 37: The method of any one of Embodiments 32 to 36, further comprising administering an additional therapy to the subject.

Embodiment 38: The method of any one of Embodiments 32 to 37, wherein the subject is a human.

Embodiment 39: Use of the antibody or antigen-binding fragment of any one of Embodiments 1 to 24 in reducing IL-7 signaling and/or TSLP signaling.

Embodiment 40: Use of the antibody or antigen-binding fragment of any one of Embodiments 1 to 24 for the preparation of a medicament for reducing IL-7 signaling and/or TSLP signaling.

Embodiment 41: Use of the antibody or antigen-binding fragment of any one of Embodiments 1 to 24 in reducing autoimmunity or inflammation.

Embodiment 42: Use of the antibody or antigen-binding fragment of any one of Embodiments 1 to 24 for the preparation of a medicament for reducing autoimmunity or inflammation.

Embodiment 43: Use of the antibody or antigen-binding fragment of any one of Embodiments 1 to 24 in treating an autoimmune or inflammatory disease associated with IL-7 and/or TSLP.

Embodiment 44: Use of the antibody or antigen-binding fragment of any one of Embodiments 1 to 24 for the preparation of a medicament for treating an autoimmune or inflammatory disease associated with IL-7 and/or TSLP.

Embodiment 45: The use of Embodiment 43 or 44, wherein the autoimmune or inflammatory disease is UC.

The practice of the invention employs, unless otherwise indicated, conventional techniques in molecular biology, cell biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) MOLECULAR CLONING: A LABORATORY MANUAL, Cold Spring Harbor Laboratory Press; Sambrook et al. (1989), MOLECULAR CLONING: A LABORATORY MANUAL, Second Edition, Cold Spring Harbor Laboratory Press; Sambrook et al. (2001) MOLECULAR CLONING: A LABORATORY MANUAL, Cold Spring Harbor Laboratory Press, Cold Spring Harbor, NY; Ausubel et al., CURRENT PROTOCOLS IN MOLECULAR BIOLOGY, John Wiley & Sons (1987 and annual updates); CURRENT PROTOCOLS IN IMMUNOLOGY, John Wiley & Sons (1987 and annual updates); Gait (ed.) (1984) OLIGONUCLEOTIDE SYNTHESIS: A PRACTICAL APPROACH, IRL Press; Eckstein (ed.) (1991) OLIGONUCLEOTIDES AND ANALOGUES: A PRACTICAL APPROACH, IRL Press; Birren et al. (eds.) (1999) GENOME ANALYSIS: A LABORATORY MANUAL, Cold Spring Harbor Laboratory Press; Borrebaeck (ed.) (1995) ANTIBODY ENGINEERING, Second Edition, Oxford University Press; Lo (ed.) (2006) ANTIBODY ENGINEERING: METHODS AND PROTOCOLS (METHODS IN MOLECULAR BIOLOGY); Vol. 248, Humana Press, Inc; Harlow et al., Antibodies: A Laboratory Manual, (Cold Spring Harbor Laboratory Press, 2nd ed. 1988); Hammerling et al., in: MONOCLONAL ANTIBODIES AND T-CELL HYBRIDOMAS 563 681 (Elsevier, N.Y., 1981); each of which is incorporated herein by reference in its entirety.

### 7.9 Examples

The examples provided below are for purposes of illustration only, which are not intended to be limiting unless otherwise specified. Thus, the invention should in no way be construed as being limited to the following examples, but rather, should be construed to encompass any and all variations which become evident as a result of the teachings provided herein.

Briefly, studies described below included the generation and characterization of candidate anti-IL-7Ra antibodies. In particular, candidate antibody cmAb011 and its humanized versions were found to bind to human IL-7Ra with high affinity and to inhibit IL-7 signaling with strong potency. Indeed, cmAb011 and its humanized versions blocked IL-7-induced T cell proliferation to a greater extent than the benchmark antibodies ADX-914 (“Tab1”; heavy chain: SEQ ID NO:46; and light chain: SEQ ID NO:45) and OSE127 (“Tab2”; heavy chain: SEQ ID NO:44; and light chain: SEQ ID NO: 43) did, and provided a better in vivo protection in the IL-7 driven mouse colitis model.

### Example 1: Immunization, Hybridoma Generation and Screening

Methods: For production of anti-IL-7Ra antibodies, Balb/c and SJL mice were immunized by several strategies based on different combinations of immunogens. All the materials mentioned below were used as immunogens. First, human Fc-tag IL-7Ra protein was generated by fusing the Fc tag to the N-terminus of human IL-7Ra (SEQ ID NO:1); second, a vector containing the sequence encoding human IL-7Ra (SEQ ID NO:1) was generated, then used for gene immunization and lentivirus packaging; and third, HEK-293F cells were infected with lentivirus to generate 293F-human IL-7Ra cells with stable expression of human IL-7Ra. In particular, cynomolgus his-tag IL-7Ra protein was generated by fusing a 6×his tag to the N-terminus of cynomolgus IL-7Ra (SEQ ID NO: 2), which was then administered as boost. Serum titers were examined for the first round of screening, and antibodies from mice with a strong immune response against IL-7Ra ECD (aa 21-239 of SEQ ID NO:1) were selected for hybridoma generation. The selected monoclonal antibodies were then re-screened for their binding affinities to IL-7Ra using ELISA, Acumen or FACS and for their activities in blocking IL-7 binding to cell membrane IL-7Ra using receptor binding assay.

### Example 2: Generation of Chimeric Antibodies

Methods: From the hybridoma screening, several chimeric antibodies were expressed and purified. The vectors expressing the heavy chains and light chains of the chimeric antibodies were constructed. The heavy chain expression vectors contained the coding sequences for the heavy chain variable domains of the chimeric antibodies linked to the coding sequence for the heavy chain constant region of human IgG4 S228P. Likewise, the light chain expression vectors contained the coding sequences for the light chain variable domains of the chimeric antibodies and the coding sequence for the constant region of k light chain.

### Example 3: Binding of Candidate Chimeric Antibodies to IL-7Ra as Measured by ELISA

Methods: The binding of the candidate chimeric anti-IL-7Ra antibodies to human and cynomolgus Fc-tag IL-7Ra protein was measured by ELISA, along with hIgG1 and hIgG4 as negative controls and reference anti-IL-7Ra antibodies Tab1 and Tab2. 96-well ELISA plates were coated with human Fc-tag IL-7Ra protein in PBS overnight at 4° C. Candidate chimeric anti-IL-7Ra antibodies were added to the wells and co-incubated with the coating protein at 37° C. for 1 hour, followed by the addition of secondary HRP labelled antibody. After incubation at 37° C. for 1 more hour, TMB substrate was added to wells and incubated at RT for 15 min. 1 N HCl was added to terminate the reactions. The absorbance at 450 nm was measured with a microplate reader.

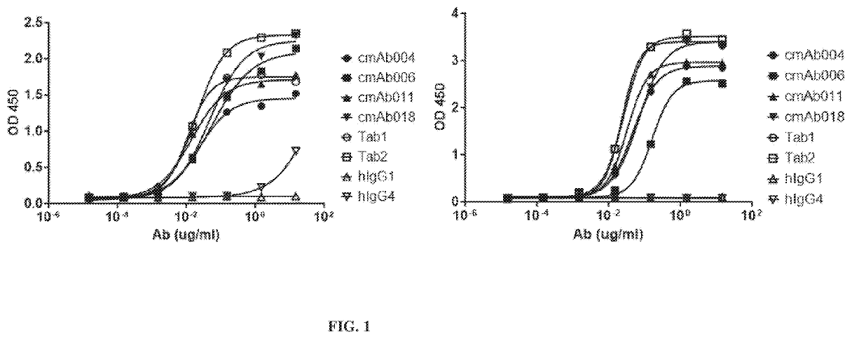

Results and conclusions: As shown in FIG. 1, candidate chimeric anti-IL-7Ra antibodies, including cmAb011, bound to human and cynomolgus IL-7Ra protein with high affinity, comparable to Tab1 and Tab2.

### Example 4: Binding of Candidate Chimeric Antibodies to IL-7Ra-Expressing Cells as Measured by Flow Cytometry

Methods: The binding affinities of candidate chimeric anti-IL-7Ra antibodies to human and cynomolgus IL-7Ra expressed on cell membrane were measured by flow cytometry, along with hIgG1 and/or hIgG4 as negative controls and reference anti-IL-7Ra antibodies Tab1 and/or Tab2. CHOK1-human IL-7Ra and CHOK1-cyno IL-7Ra cells were generated by infection with lentivirus mentioned above. IL-7Ra-expressing cells were seeded, resuspended, and incubated with the candidate chimeric antibodies at 4° C. for 1 hour. Cells were then washed with cold FACS buffer (PBS+2% FBS), resuspended, and incubated with secondary antibody at 4° C. for 1 hour.

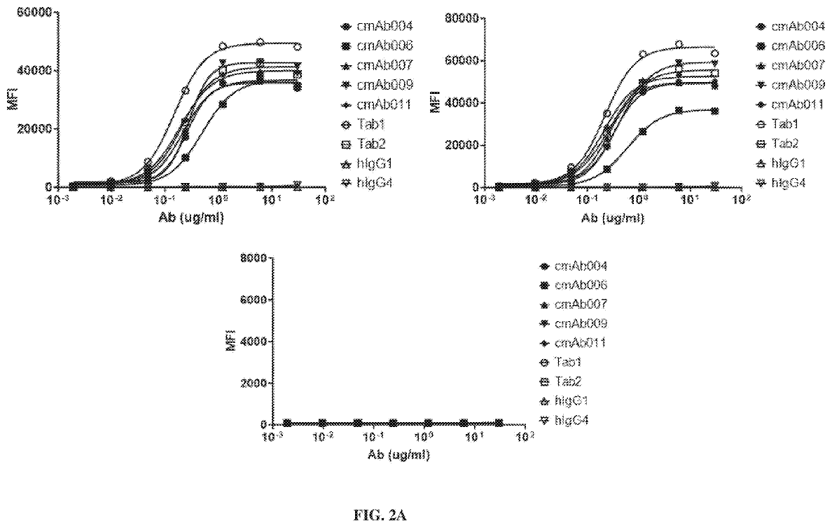

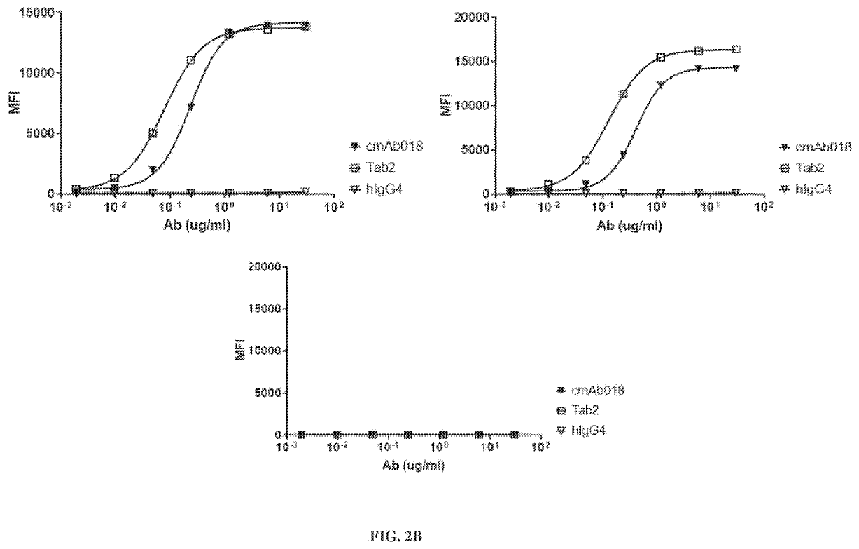

Results and conclusions: As shown by the concentration-MFI (“Median Fluorescence Intensity”) curves in FIGS. 2A-2B, candidate chimeric anti-IL-7Ra antibodies, including cmAb011, bound to CHOK1-human IL-7Ra (upper left) and CHOK1-cyno IL-7Ra (upper right) in a dose-dependent manner, but not to CHOK1-blank cells (lower), which was comparable to Tab1 and Tab2.

### Example 5: Blocking Activities of Candidate Chimeric Antibodies on IL-7 Binding to Cell Membrane

Methods: The blocking activities of candidate chimeric antibodies on IL-7 binding to human IL-7Ra expressed on cell membrane were measured by flow cytometry, along with hIgG1 and/or hIgG4 as negative controls and reference anti-IL-7Ra antibodies Tab1 and/or Tab2. CHOK1-human IL-7Ra cells were suspended in ice cold FACS buffer (1×PBS with 2% FBS) and seeded into the 96-well round-bottom plates at a density of 5×10⁴ cells/well. The plates were centrifuged at 300×g for 5 minutes, and the supernatant was discarded. Cells were resuspended with 50 μl of diluted antibodies and incubated at 4° C. for 15 minutes. 50 μL of hIL-7-his (final conc.: 0.1 μg/ml, Gene script) was added into each well and mixed. The plates were incubated for 1 hour at 4° C. After incubation, cells were washed 3 times with 200 μL/well of ice cold FACS buffer. 100 μL of diluted secondary antibody (1:1000 dilutions, Alexa Fluor® 488 anti-His Tag, BioLegend) was added to each well and incubated at 4° C. for 1 hour. After washing of cells for 3 times with 200 μL/well of ice cold FACS buffer, cells were resuspended in 100 μL/well of ice cold FACS buffer and analyzed by BD FACSCanto II.

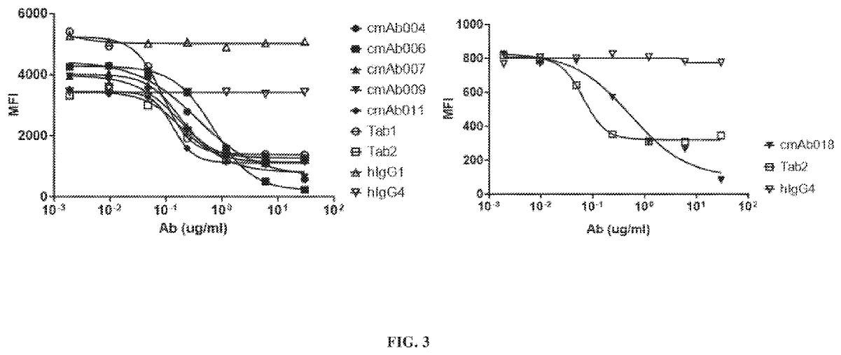

Results and conclusions: As shown by the concentration-MFI curves in FIG. 3, candidate chimeric anti-IL-7Ra antibodies, including cmAb011, potently blocked IL-7 from binding to the surface of CHOK1-human IL-7Ra cells in a dose-dependent manner, which was comparable to Tab 1 and Tab2.

### Example 6: Blocking Activities of Candidate Chimeric Antibodies on IL-7-Induced STAT5 Activation

Methods: The function of the candidate chimeric antibodies in blocking IL-7-induced STAT5 activation (phosphorylation) was measured in PBMC by flow cytometry, along with hIgG1 and/or hIgG4 as negative controls and reference anti-IL-7Ra antibodies Tab1 and/or Tab2. Frozen human PBMCs (Allcells) were suspended at a density of 2×10⁶ cells/mL in serum-free RPMI 1640 medium (Gibco) and 100 μL/well of cell suspension was seeded into 96-well plates. Diluted antibodies were added to cells in 50 L/well and incubated at 37° C. for 15 minutes. 50 L/well of hIL-7-his (final conc.: 5 ng/mL) was added to the cells, mixed, and incubated at 37° C. for 15 minutes. After incubation, cells were washed once with ice cold FACS buffer to stop the reaction. The plates were centrifuged at 500×g for 5 minutes at 4° C. and cells were treated for 15 minutes with 100 μL/well of cold cytofix/cytoperm solution. The plates were washed once with Perm/Wash buffer (BD Perm/Wash™) and centrifuged at 500×g for 5 minutes at 4° C. Cells were then stained with Alexa Fluor® 488 Mouse Anti-Human CD3 antibody (BD Bioscience) at 4° C. for 30 minutes. Cells were washed once with Perm/Wash buffer and centrifuged at 500×g for 5 minutes at 4° C. Cells were then permeabilized with BD Perm Buffer III (BD Phosflow™) for 30 minutes at 4° C. After permeabilization, the plates were washed once with FACS buffer and centrifuged at 500×g for 5 minutes at 4° C. Cells were stained with Alexa Fluor® 647 Mouse Anti-Stat5 (pY694) antibody (BD Phosflow™), and incubated at 37° C. for 30 minutes. PBMCs were washed 3 times with 200 μL/well of ice cold FACS buffer and then resuspended in 100 μL/well of ice cold FACS buffer. PBMCs were analyzed by BD FACSCanto II. Untreated PBMCs were assessed as the background signal.

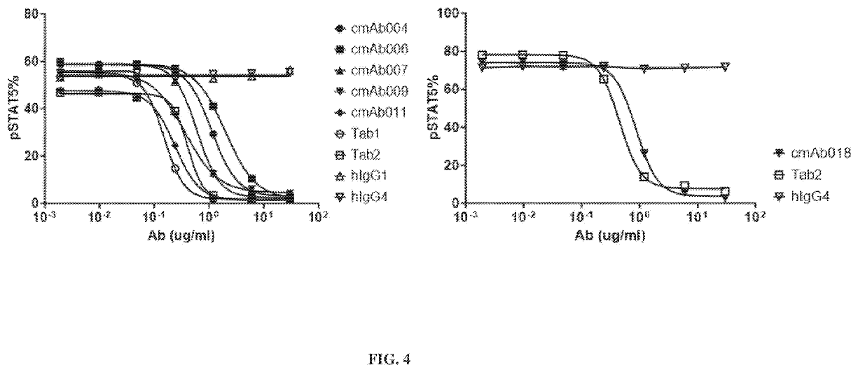

Results and conclusions: As shown by the concentration-pSTAT5% curves in FIG. 4, candidate chimeric anti-IL-7Ra antibodies, including cmAb011, potently blocked IL-7-induced STAT5 phosphorylation in a dose-dependent manner, which was comparable to Tab1 and/or Tab2.

### Example 7: Blocking Activities of Candidate Chimeric Antibodies on IL-7-Induced T Cell Proliferation

Methods: The function of candidate chimeric anti-IL-7Ra antibodies in blocking IL-7-induced T cell proliferation was measured using various donors' PBMCs by flow cytometry, along with hIgG1 and/or hIgG4 as negative controls and reference anti-IL-7Ra antibodies Tab1 and Tab2. 96-well plates (Corning) were coated with 0.01 μg/mL anti-CD3 antibody (OKT3) at 4° C. overnight. PBMCs were placed in a 37° C. water bath for rapid thawing and resuspended with RPMI 1640 medium (Gibco) supplemented with 10% FBS (BIOSUN). PBMCs were seeded in a 25 cm culture dish (Corning) and incubated overnight at 37° C., 5% CO₂. PBMCs were stained with 2 μM of CFSE (Invitrogen). After staining, PBMCs were washed 3 times with PBS (Corning Cellgro). PBMCs were resuspended with medium and seeded into the plates at a density of 2×10⁵ cells/well. 50 μL/well of diluted antibodies (final conc.: 15 μg/mL) was added to the cells, and the plates were incubated at 37° C. for 1 hour. After incubation, 50 μL/well of hIL-7-his (final conc.: 12.5 ng/mL) was added into the plates, mixed, and incubated at 37° C. for 3 days. After incubation of 3 days, PBMCs were then resuspended with 100 μL/well of antibodies mixture (anti-CD3 or anti-CD4/CD8 antibodies: 5 μL/test) and incubated at 4° C. for 1 hour in the dark. The PBMCs were washed with ice cold FACS buffer and centrifuged at 500×g for 5 minutes. All stained samples were resuspended with 200 μL/well of FACS buffer and analyzed by BD FACSCanto II.

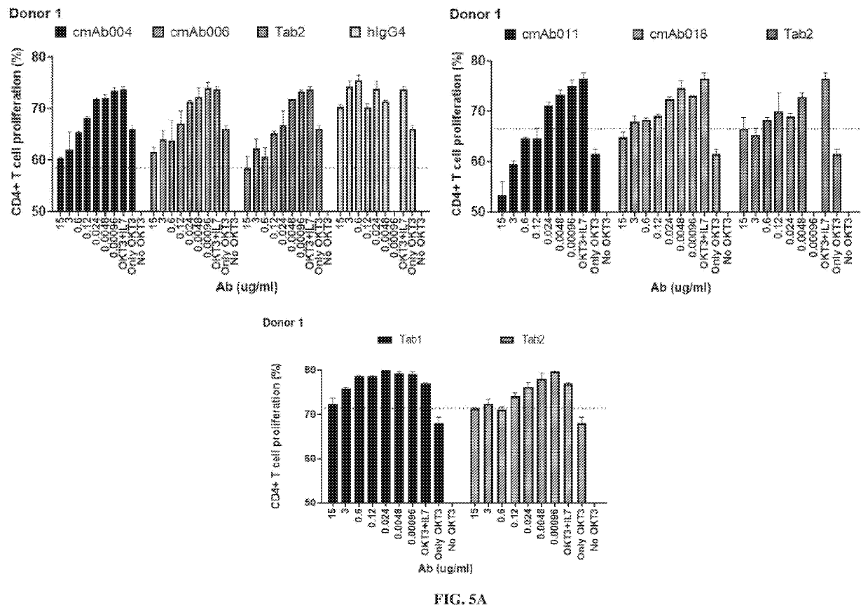

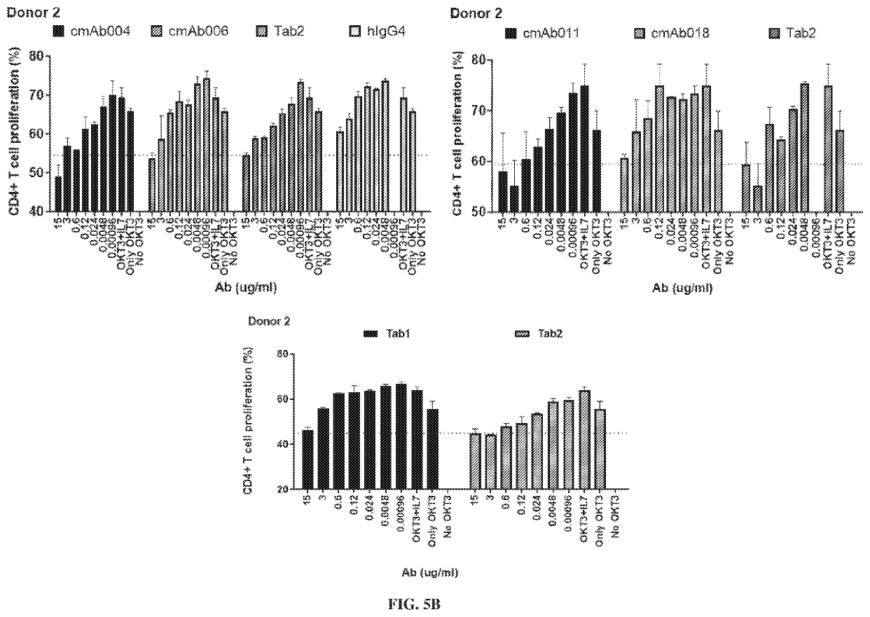

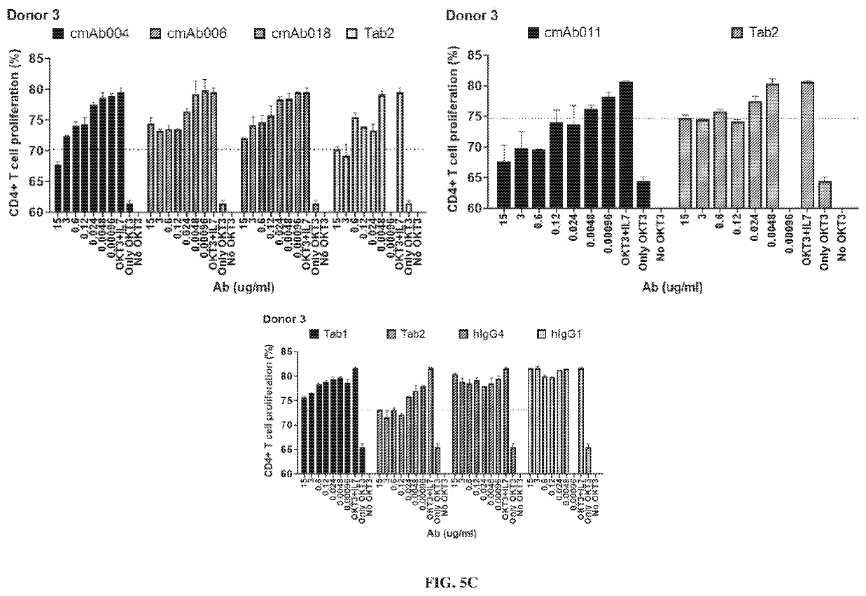

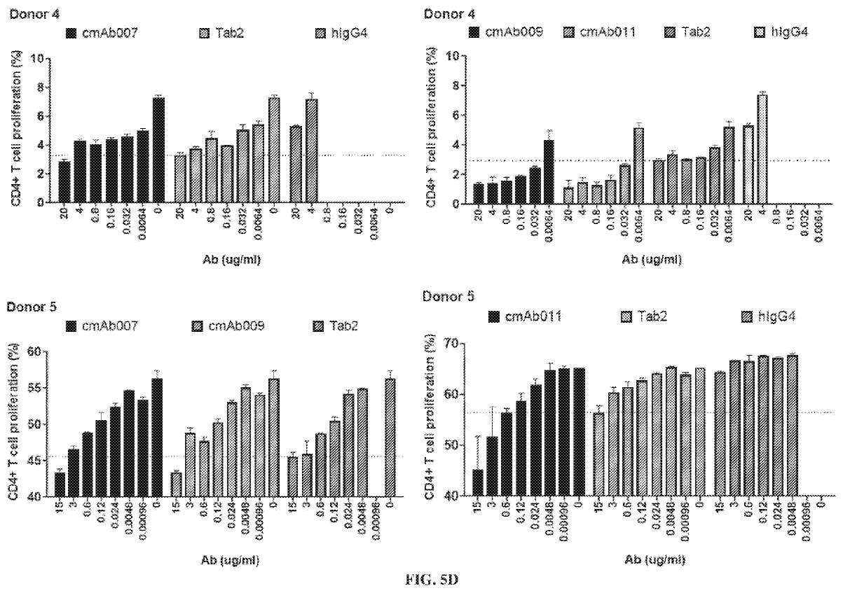

Results and conclusions: As shown in FIGS. 5A-5D, all candidate chimeric antibodies, including cmAb011, potently blocked IL-7-induced T cell proliferation in a dose-dependent manner. Unexpectedly, cmAb011 consistently showed greater blockage of IL-7-induced T cell proliferation than Tab1, Tab2, and other candidate chimeric antibodies did.

### Example 8: Humanization of cmAb011

Methods: Major risky hotspots, including unpaired cysteine residues, N-glycosylation site, and deamination site within the CDRs, were analyzed. No hotspot was found in CDRs of cmAb011. Based on VH/VL CDR canonical structures and homology model, the best human germline framework acceptors were selected. The human J-region was selected based on the best sequence homology. According to the 3D structure of homology modeling, residues within 5 Å distance of CDRs were selected as a potential backmutation sites because the exposed residues might be involved in antigen binding directly and the buried residues might be important for maintaining the CDR conformation. The VH/VL interfaces were analyzed and backmutations were introduced if issues were identified. The importance of individual backmutations was determined by in silico analysis and a panel of engineered antibodies containing different combinations of single backmutation was designed.

Sequences of the VLs and VHs of humanized 011 are provided in Table 3. Identifiers of the specific humanized antibody VL/VH combinations are provided in Table 4.

### Example 9: Binding of the Candidate Humanized Antibodies to IL-7Ra-Expressing Cells as Measured by Flow Cytometry

Methods: The binding affinities of the chimeric anti-IL-7Ra antibody (cmAb011) and the humanized anti-IL-7Ra antibodies (HuAb001-020) to human and cynomolgus IL-7Ra expressed on cell membrane were measured by flow cytometry, along with isotype hIgG4 antibody as negative control and reference anti-IL-7Ra antibodies Tab1 and Tab2, using similar method as described in Example 4.

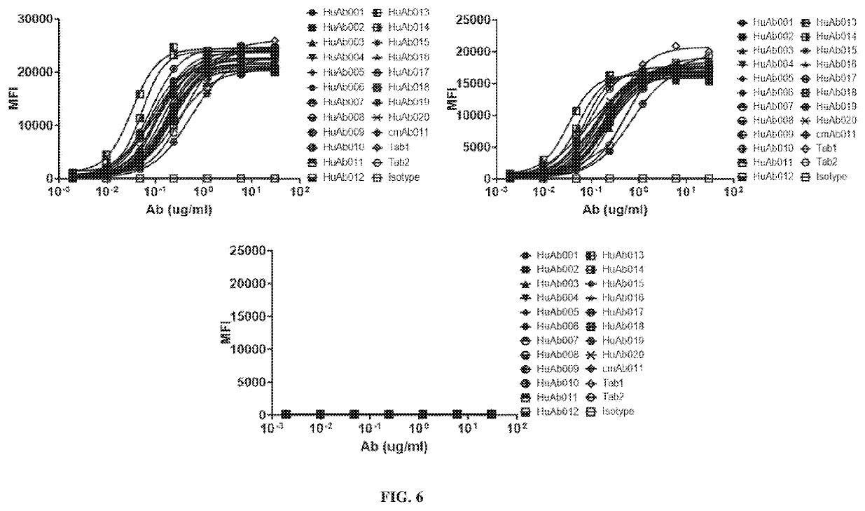

Results and conclusions: As shown by the concentration-MFI curves in FIG. 6 and Table 5, comparable to cmAb011, Tab1 and Tab2, all candidate humanized antibodies bound to CHOK1-human IL-7Ra (upper left) and CHOK1-cyno IL-7Ra (upper right) in a dose-dependent manner, but not to CHOK1-blank cells (lower). The EC₅₀ of the candidate humanized antibodies binding to CHOK1-human IL-7Ra ranged from 0.03265 μg/mL to 0.5359 μg/mL, and the EC₅₀ that to CHOK1-cyno IL-7Ra ranged from 0.03246 μg/mL to 0.6873 μg/mL.

TABLE 5
The EC₅₀ of humanized antibodies binding
to IL-7Ra measured by FACS

| | CHOK1-Human | | | CHOK1-Cyno | |
|---|---|---|---|---|---|
| | IL-7Ra cells | | | IL-7Ra cells | |
| Humanized | EC₅₀ | Top | Humanized | EC₅₀ | Top |
| antibody | (μg/mL) | (MFI) | antibody | (μg/mL) | (MFI) |
| HuAb001 | 0.1702 | 22312 | HuAb001 | 0.1674 | 16024 |
| HuAb002 | 0.1146 | 22643 | HuAb002 | 0.1263 | 15879 |
| HuAb003 | 0.2096 | 21745 | HuAb003 | 0.239 | 16057 |
| HuAb004 | 0.1996 | 21512 | HuAb004 | 0.2059 | 16151 |
| HuAb005 | 0.1883 | 22296 | HuAb005 | 0.2069 | 16870 |
| HuAb006 | 0.1775 | 21551 | HuAb006 | 0.1914 | 16699 |
| HuAb007 | 0.2088 | 21133 | HuAb007 | 0.2437 | 17145 |
| HuAb008 | 0.1714 | 20394 | HuAb008 | 0.2211 | 17627 |
| HuAb009 | 0.07194 | 20852 | HuAb009 | 0.07109 | 16559 |
| HuAb010 | 0.1523 | 20623 | HuAb010 | 0.1753 | 17471 |
| HuAb011 | 0.2563 | 20536 | HuAb011 | 0.2414 | 17736 |
| HuAb012 | 0.1585 | 20302 | HuAb012 | 0.1647 | 16984 |
| HuAb013 | 0.03265 | 24363 | HuAb013 | 0.03246 | 16332 |
| HuAb014 | 0.05285 | 23877 | HuAb014 | 0.05335 | 16661 |
| HuAb015 | 0.1579 | 24107 | HuAb015 | 0.1891 | 17423 |
| HuAb016 | 0.1134 | 23713 | HuAb016 | 0.1279 | 17082 |
| HuAb017 | 0.07781 | 24646 | HuAb017 | 0.08906 | 17721 |
| HuAb018 | 0.1053 | 24061 | HuAb018 | 0.09881 | 18400 |
| HuAb019 | 0.5359 | 22837 | HuAb019 | 0.6873 | 17990 |
| HuAb020 | 0.1146 | 22499 | HuAb020 | 0.1238 | 17589 |
| cmAb011 | 0.08415 | 22405 | cmAb011 | 0.1344 | 18364 |
| Tab1 | 0.3098 | 26032 | Tab1 | 0.2894 | 20759 |
| Tab2 | 0.3868 | 24336 | Tab2 | 0.5103 | 19115 |
| IgG4 | ~6.804 | 73 | IgG4 | ~32.23 | ~93.67 |

### Example 10: Blocking Activities of Humanized Antibodies on IL-7 Binding to Cell Membrane

Methods: The blocking activities of the anti-IL-7Ra chimeric antibody (cmAb011) and candidate humanized antibodies (HuAb001-009, 011, 013, 015) on IL-7 binding to human IL-7Ra expressed on cell membrane were measured by flow cytometry, along with isotype control antibody as negative control and reference anti-IL-7Ra antibodies Tab1 and Tab2, using methods similar to those described in Example 5.

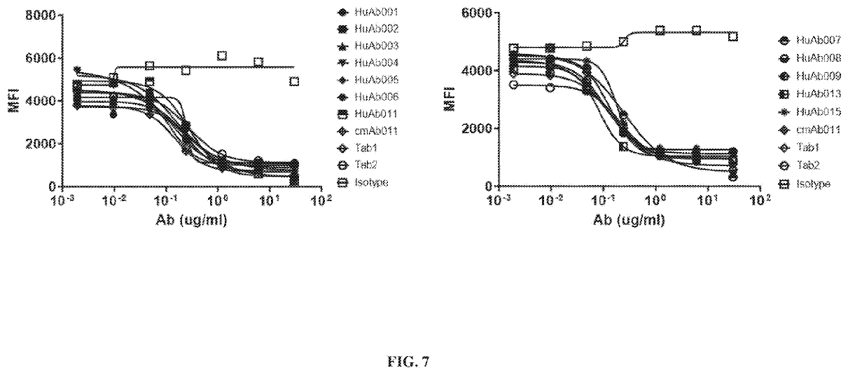

Results and conclusions: As shown by the concentration-MFI curves in FIG. 7, comparable to cmAb011, Tab1 and Tab2, all candidate humanized antibodies potently blocked IL-7 binding to the surface of CHOK1-human IL-7Ra cells in a dose-dependent manner.

### Example 11: Binding of the Humanized Antibodies to IL-7Ra as Measured by Biacore

Methods: The binding affinities of the anti-IL-7Ra chimeric antibody (cmAb011) and candidate humanized antibodies were further measured by surface plasmon resonance (SPR) technology with Biacore 8K, along with isotype control antibody as negative control and reference anti-IL-7Ra antibodies Tab1 and Tab2. The tested antibodies were immobilized on CM-5 chip (GE), captured on the sensor chip through Fc capture method, flow rate (10 μL/min), contact time (60 s). Human his-tag IL-7Ra protein (1:1 diluted for 8 doses from 200 nM) was used as the analyte, flow rate (30 μL/min), contact time (180 s), dissociation (400 s). Regeneration: both flow cells 1&2 path, injection 10 mM glycine-HCl, pH1.5, flow rate (30 μL/min), contact time (30 s). Regeneration: both flow cells 1&2 path, injection 10 mM glycine-HCl, pH1.5, flow rate (30 μL/min), contact time (30 s). KD value was calculated using Data Analysis evaluation software version 1.0 of Biacore T200 and a 1:1 binding model was applied for curve fitting.

Results and conclusions: As shown in Table 6, Biacore measurements demonstrated that all candidate humanized antibodies bound to IL-7Ra with high affinities, comparable to that of cmAb011, Tab1, and Tab2. The K_{D} of the candidate humanized antibodies ranged from 1.18E-10M to 2.14E-10M.

TABLE 6
The K_{D} of huAbs to IL-7Ra as measured by Biacore

| Antibody | Chi² (RU²) | ka (1/Ms) | kd (1/s) | K_{D} (M) |
|---|---|---|---|---|
| HuAb003 | 6.54E−02 | 1.61E+06 | 3.42E−04 | 2.12E−10 |
| HuAb004 | 1.35E−01 | 1.60E+06 | 2.90E−04 | 1.81E−10 |
| HuAb005 | 2.24E−01 | 1.11E+06 | 2.38E−04 | 2.14E−10 |
| HuAb008 | 1.35E−01 | 2.64E+06 | 3.10E−04 | 1.18E−10 |
| HuAb009 | 1.01E−01 | 1.42E+06 | 2.67E−04 | 1.88E−10 |
| HuAb013 | 1.96E−01 | 1.63E+06 | 2.92E−04 | 1.80E−10 |
| cmAb011 | 9.47E−02 | 1.64E+06 | 2.34E−04 | 1.43E−10 |
| Tab1 | 2.59E−01 | 1.15E+06 | 2.97E−04 | 2.58E−10 |
| Tab2 | 7.29E−02 | 6.56E+05 | 2.52E−04 | 3.85E−10 |

### Example 12: Blocking Activities of Candidate Humanized Antibodies on IL-7-Induced STAT5 Activation

Methods: The function of the chimeric antibody (cmAb011) and candidate anti-IL-7Ra humanized antibodies in blocking IL-7-induced STAT5 activation (as indicated by its phosphorylation, or pSTAT5) was measured in PBMCs by flow cytometry, along with isotype control antibody as negative control and reference anti-IL-7Ra antibodies Tab1 and Tab2, using methods similar to those described in Example 6.

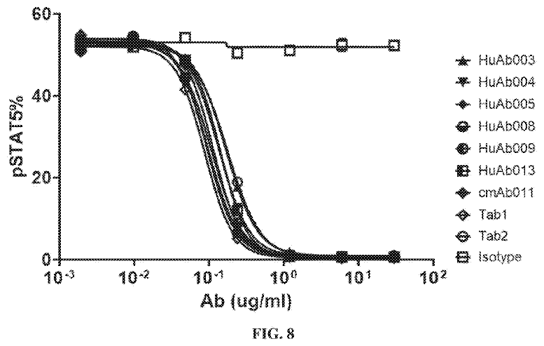

Results and conclusions: As shown by the concentration-pSTAT5% curves in FIG. 8, comparable to cmAb011, Tab1 and Tab2, all candidate humanized antibodies potently blocked IL-7-induced STAT5 activation in a dose-dependent manner.

### Example 13: Blocking Activities of Candidate Humanized Antibodies on IL-7-Induced T Cell Proliferation

Methods: The function of the chimeric anti-IL-7Ra antibody (cmAb011) and candidate anti-humanized IL-7Ra antibodies in blocking IL-7-induced T cell proliferation was measured using various donors' PBMCs by flow cytometry, along with hIgG1 and hIgG4 as negative controls and reference anti-IL-7Ra antibodies Tab1 and Tab2, using methods similar to those described in Example 7, except the OKT3 was used at 0.0025 μg/mL for studies on donor 4, 5, 6. The percentages of IL-7-induced proliferating CD4+ T cells were calculated using that of untreated “Only OKT3” group as the baseline.

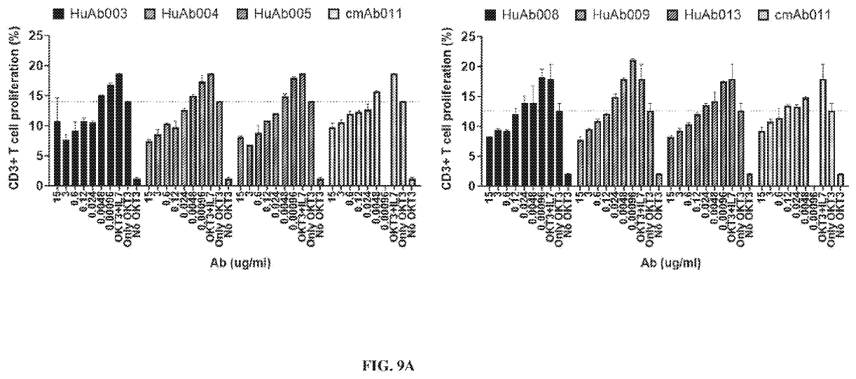

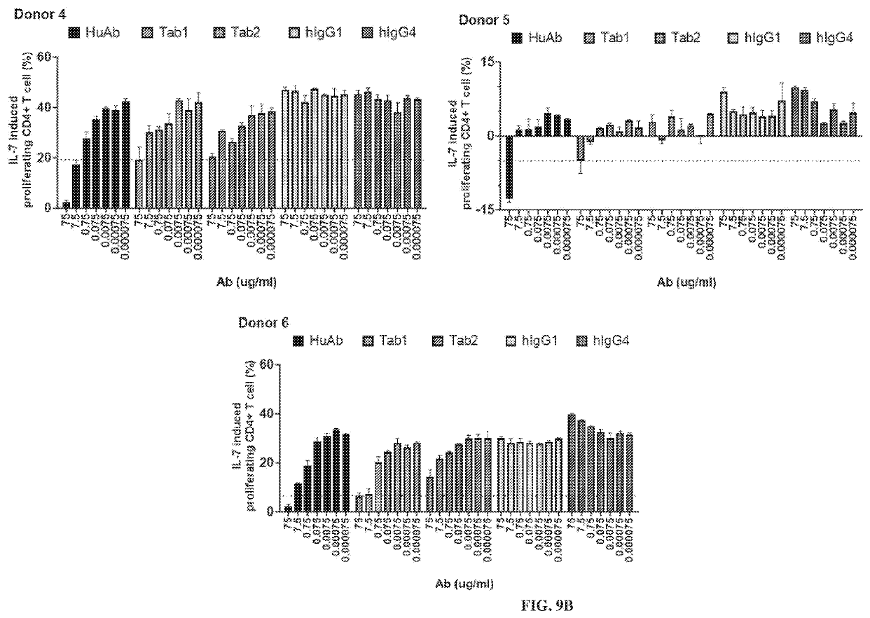

Results and conclusions: Representative results are shown in FIGS. 9A-9B. As shown in FIG. 9A, comparable to cmAb011, all candidate humanized antibodies potently blocked IL-7-induced T cell proliferation in a dose-dependent manner. Furthermore, as shown in FIG. 9B, a representative humanized antibody consistently showed greater blockage of IL-7-induced T cell proliferation than Tab1 and Tab2 did.

### Example 14: Protection from High Dose IL-7 Driven Colitis by Candidate Humanized Antibodies in Humanized Mice

Methods: 56 M-NSG mice were reconstituted with 5E6 human PBMC (5E6 in 200 μL) through the tail vein on the day-16. After 14 days of PBMC reconstitution (day-2), blood was collected for hCD3 detection. On day-1, body weight and the ratio of hCD3 to hCD45+mCD45 were comprehensively considered for grouping. The candidate humanized antibody (HuAb), isotype control, or reference antibody (Tab2) were administered on day-1, day 2, and day 5 by intraperitoneal injection at a dose of 5 mg/kg. TNBS modeling was conducted on day 0. Mice were fasted for 24 hours but provided with water. On the day of modeling, the mice were anesthetized with isoflurane, and 100 μL TNBS (2.5 mg, 50% EtOH) was delivered by inserting the needle (covered with a lubricated 3.5F soft silicon catheter) carefully into the mouse anus to a depth of 3-4 cm, and slowly pushing in the medication, avoiding damaging the intestinal wall during the process. An equal volume of 50% EtOH was delivered using the same procedure in the sham control. 1 μg hIL-7 was intravenously injected on day 7. During the study, weight and enteritis score were monitored daily. The differences between the HuAb group and the Tab2 group were calculated using Two Way ANOVA with data from day 7 and found to be statistic significant (*p<0.05, **p<0.01).

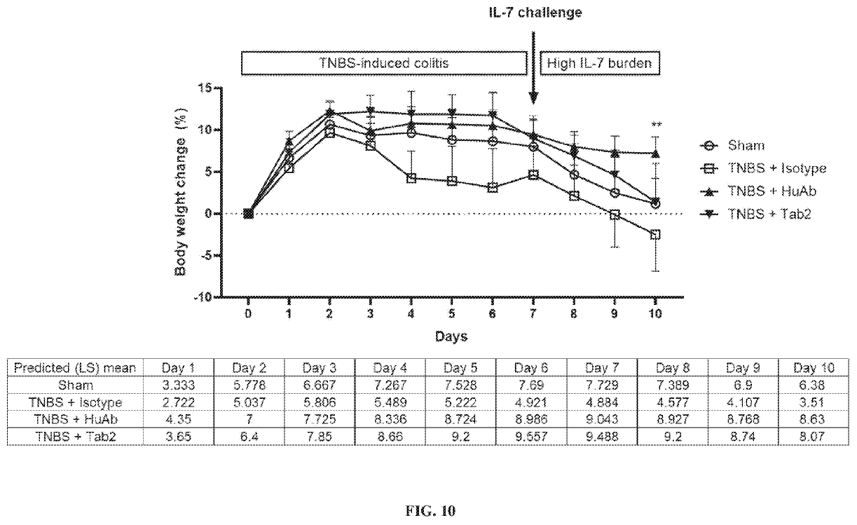

Results and conclusions: Body weight and the predicted Least Square (LS) mean are provided in FIG. 10. As shown, 0-2 days after modeling, the body weight in all groups still maintained an upward trend. 3-7 days after modeling, the body weight in isotype treated group decreased compared to that of the sham group, while the HuAb group and Tab2 group showed protection of body weight loss. On day 7 after TNBS modeling, a high dose of hIL-7 was injected intravenously, which resulted in weight loss in all groups except for the HuAb group. As such, the candidate humanized antibody demonstrated in vivo protection from high dose IL-7 driven mouse colitis, which was significantly better compared with Tab2.

### Example 15: Blocking Activities of the Candidate Humanized Antibodies on TSLP Induced TARC Secretion

Methods: The function of the candidate humanized antibody in blocking TSLP induced TARC secretion was measured in treated PBMCs supernatant by ELISA, along with hIgG1 and hIgG4 as negative controls (high doses only) and reference anti-IL-7Ra antibodies Tab1 and Tab2. The frozen human PBMCs were thawed rapidly at 37° C. PBMCs were resuspended with RPMI 1640 medium (Gibco) with 10% FBS (BIOSUN) and seeded into the plates (Corning) at a density of 5×10⁵ cells/well. 50 μL/well of diluted antibodies (final conc. ranging from 0.015 μg/mL to 150 μg/mL) and 50 μL of TSLP (final conc.: 40 ng/mL) were added into the plates, mixed, and incubated at 37° C. for 24 hours. The plates were centrifuged at 400×g for 5 minutes and 80 μL of supernatant was collected for TARC detection according to TARC Matched ELISA Antibody Pair Set Kit (SINO BIOLOGICAL) procedure.

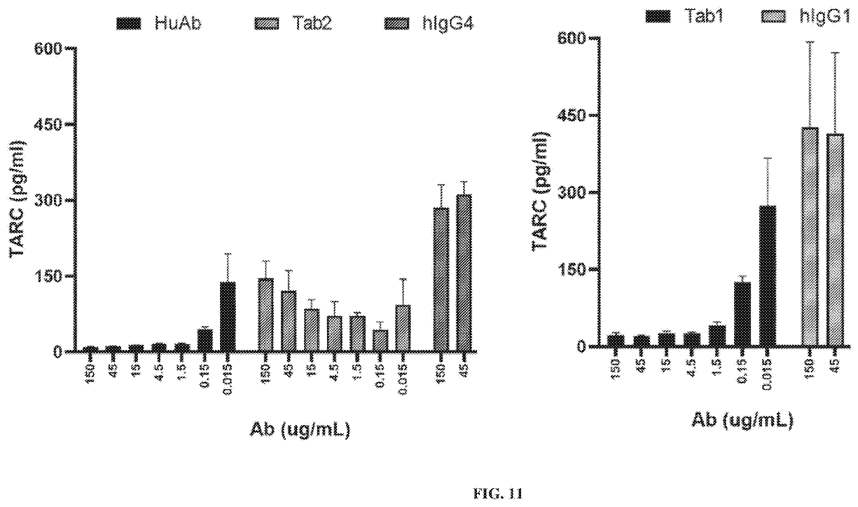

Results and conclusions: As shown in FIG. 11, the candidate humanized antibody HuAb potently blocked TSLP induced TARC secretion in a dose-dependent manner.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Unless the context indicates otherwise, it is specifically intended that the various features described herein can be used in any combination. Unless otherwise defined, all technical and scientific terms used herein have the same meaning as commonly understood by one of ordinary skill in the art to which this disclosure belongs. Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

What is claimed is:

1. An antibody or antigen-binding fragment thereof that specifically binds human IL-7Ra, comprising:
(1) as defined by Kabat,
(a) a light chain variable region (VL) comprising VL CDR1, VL CDR2, VL CDR3 having the amino acid sequences of SEQ ID NOs: 8, 9, and 10, respectively; or a variant thereof having up to about 5 amino acid substitutions, additions, and/or deletions in the VL CDRs; and/or
(b) a heavy chain variable region (VH) comprising VH CDR1, VH CDR2, VH CDR3 having the amino acid sequences of SEQ ID NOs: 12, 14 and 15, respectively; or a variant thereof having up to about 5 amino acid substitutions, additions, and/or deletions in the VH CDRs; or
(2) as defined by Chothia,
(a) a VL comprising VL CDR1, VL CDR2, VL CDR3 having the amino acid sequences of SEQ ID NOs: 8, 9, and 10, respectively; or a variant thereof having up to about 5 amino acid substitutions, additions, and/or deletions in the VL CDRs; and/or
(b) a VH comprising VH CDR1, VH CDR2, VH CDR3 having the amino acid sequences of SEQ ID NOs: 11, 13, and 15, respectively; or a variant thereof having up to about 5 amino acid substitutions, additions, and/or deletions in the VH CDRs.

2. The antibody or antigen-binding fragment of claim 1, comprising VL CDR1, VL CDR2, VL CDR3, VH CDR1, VH CDR2 and VH CDR3 having the amino acid sequences of SEQ ID NOs: 8, 9, 10, 12, 14 and 15, respectively, as defined by Kabat.

3. The antibody or antigen-binding fragment of claim 1, comprising VL CDR1, VL CDR2, VL CDR3, VH CDR1, VH CDR2 and VH CDR3 having the amino acid sequences of SEQ ID NOs: 8, 9, 10, 11, 13, and 15, respectively, as defined by Chothia.

4. An antibody or antigen-binding fragment thereof that specifically binds human IL-7Ra, comprising:
(a) a VL having at least 85%, at least 90%, at least 95%, at least 98%, or 100% sequence identity to the amino acid sequence of SEQ ID NO:18; and/or
(b) a VH having at least 85%, at least 90%, at least 95%, at least 98%, or 100% sequence identity to the amino acid sequence of SEQ ID NO:19.

5. The antibody or antigen-binding fragment of claim 4 comprising a VL and a VH having the amino acid sequences of SEQ ID NOs: 18 and 19, respectively.

6. An antibody or antigen-binding fragment thereof that specifically binds human IL-7Ra, comprising
(a) a VL comprising VL CDR1, VL CDR2, and VL CDR3 from a VL having the amino acid sequence of SEQ ID NO:18; and/or
(b) a VH comprising VH CDR1, VH CDR2, and VH CDR3 from a VH having the amino acid sequence of SEQ ID NO:19.

7. The antibody or antigen-binding fragment of any one of claims 1 to 6 that is a chimeric antibody or antigen-binding fragment, a humanized antibody or antigen-binding fragment, or a human antibody or antigen-binding fragment.

8. The antibody or antigen-binding fragment of claim 7 that is a humanized antibody or antigen-binding fragment.

9. The antibody or antigen-binding fragment of claim 8, comprising:
(a) a VL having at least 85%, at least 90%, at least 95%, at least 98%, or 100% sequence identity to an amino acid sequence selected from the group consisting of SEQ ID NOs: 20-23; and/or
(b) a VH having at least 85%, at least 90%, at least 95%, at least 98%, or 100% sequence identity to an amino acid sequence selected from the group consisting of SEQ ID NOs: 26-30.

10. The antibody or antigen-binding fragment of claim 9 comprising a VL and a VH having the amino acid sequences of (1) SEQ ID NOs: 20 and 26, respectively; (2) SEQ ID NOs: 20 and 27, respectively; (3) SEQ ID NOs: 20 and 28, respectively; (4) SEQ ID NOs: 20 and 29, respectively; (5) SEQ ID NOs: 20 and 30, respectively; (6) SEQ ID NOs: 21 and 26, respectively; (7) SEQ ID NOs: 21 and 27, respectively; (8) SEQ ID NOs: 21 and 28, respectively; (9) SEQ ID NOs: 21 and 29, respectively; (10) SEQ ID NOs: 21 and 30, respectively; (11) SEQ ID NOs: 22 and 26, respectively; (12) SEQ ID NOs: 22 and 27, respectively; (13) SEQ ID NOs: 22 and 28, respectively; (14) SEQ ID NOs: 22 and 29, respectively; (15) SEQ ID NOs: 22 and 30, respectively; (16) SEQ ID NOs: 23 and 26, respectively; (17) SEQ ID NOs: 23 and 27, respectively; (18) SEQ ID NOs: 23 and 28, respectively; (19) SEQ ID NOs: 23 and 29, respectively; or (20) SEQ ID NOs: 23 and 30, respectively.

11. The antibody or antigen-binding fragment of any one of claims 1 to 10 that is selected from the group consisting of a Fab, a Fab′, a F(ab′)₂, a Fv, a scFv, a (scFv)₂, a single domain antibody (sdAb), and a heavy chain antibody (HCAb).

12. The antibody or antigen-binding fragment of any one of claims 1 to 10 that is an IgG1 antibody, an IgG2 antibody, an IgG3 antibody, or an IgG4 antibody.

13. The antibody of claim 12 comprising a light chain constant region (CL) having at least 85% sequence identity to kappa CL (Cκ; SEQ ID NO:33).

14. The antibody of claim 12 comprising a light chain constant region (CL) having at least 85% sequence identity to lambda CL (Cλ; SEQ ID NO:34).

15. The antibody of claim 12 comprising a heavy chain constant region (CH) having at least 85% sequence identity to an amino acid sequence selected from the group consisting of SEQ ID NOs: 35-38.

16. The antibody of claim 12 that is an IgG4 antibody.

17. The antibody of claim 16, wherein the heavy chain constant region (CH) of the IgG4 antibody comprises a wildtype IgG4 CH, or comprises at least one amino acid mutation.

18. The antibody of claim 17, wherein the CH region of the IgG4 antibody has S228P substitution (SEQ ID NO:39).

19. The antibody of any one of claims 16 to 18, wherein the Fc region of the antibody is afucosylated.

20. An antibody or antigen-binding fragment thereof that competes with the antibody or antigen-binding fragment of any one of claims 1 to 19 for binding to human IL-7Ra.

21. The antibody or antigen-binding fragment of any one of claims 1 to 20 that is a bispecific antibody or a multispecific antibody.

22. The antibody or antigen-binding fragment of any one of claims 1 to 21 that is a monoclonal antibody or antigen-binding fragment.

23. The antibody or antigen-binding fragment of any one of claims 1 to 22, wherein the antibody or antigen-binding fragment:
(1) binds to human IL-7Ra with high affinity;
(2) blocks IL-7 binding to IL-7Ra;
(3) blocks IL-7-induced STAT5 phosphorylation;
(4) blocks IL-7-induced T cell proliferation;
(5) blocks TSLP induced TARC secretion; or
(6) ameliorates colitis; or any combination of (1)-(6).

24. An antibody or antigen-binding fragment thereof that specifically binds human IL-7Ra, wherein the antibody or antigen-binding fragment:
(1) binds to human IL-7Ra with high affinity;
(2) blocks IL-7 binding to IL-7Ra;
(3) blocks IL-7-induced STAT5 phosphorylation;
(4) blocks IL-7-induced T cell proliferation;
(5) blocks TSLP induced TARC secretion; or
(6) ameliorates colitis; or any combination of (1)-(6).

25. A polynucleotide encoding a polypeptide of the antibody or antigen-binding fragment of any one of claims 1 to 24.

26. A vector comprising the polynucleotide of claim 25.

27. A host cell comprising the polynucleotide of claim 25, or the vector of claim 26.

28. The host cell of claim 27, that (1) overexpresses N-acetylglucosaminyltransferase III (GnTIII), (2) lacks α-1,6-fucosyltransferase (FUT8), or (3) has a low fucose content, or any combination of (1)-(3).

29. A method of making an antibody or antigen-binding fragment thereof that specifically binds human IL-7Ra, comprising culturing the cell of claim 27 or 28 under conditions that allow expression of the antibody or antigen-binding fragment.

30. The method of claim 29 that comprises isolating the antibody or antigen-binding fragment from the culture.

31. A pharmaceutical composition comprising a therapeutically effective amount of the antibody or antigen-binding fragment of any one of claims 1 to 24, and a pharmaceutically acceptable carrier.

32. A method of reducing IL-7 signaling and/or TSLP signaling in a subject in need thereof, comprising administering to the subject a therapeutically effective amount of the antibody or antigen-binding fragment of any one of claims 1 to 24.

33. A method of reducing autoimmunity or inflammation in a subject in need thereof, comprising administering to the subject an effective amount of the antibody or antigen-binding fragment of any one of claims 1 to 24.

34. The method of any one of claims 32 to 33, wherein the subject has an autoimmune or inflammatory disease.

35. A method of treating an autoimmune or inflammatory disease associated with IL-7 and/or TSLP in a subject in need thereof, comprising administering to the subject a therapeutically effective amount of the antibody or antigen-binding fragment of any one of claims 1 to 24.

36. The method of claim 34 or 35, wherein the autoimmune or inflammatory disease is ulcerative colitis (UC).

37. The method of any one of claims 32 to 36, further comprising administering an additional therapy to the subject.

38. The method of any one of claims 32 to 37, wherein the subject is a human.

39. Use of the antibody or antigen-binding fragment of any one of claims 1 to 24 in blocking IL-7 binding to IL-7Ra.

40. Use of the antibody or antigen-binding fragment of any one of claims 1 to 24 for the preparation of a medicament for reducing IL-7 signaling and/or TSLP signaling.

41. Use of the antibody or antigen-binding fragment of any one of claims 1 to 24 in reducing autoimmunity or inflammation.

42. Use of the antibody or antigen-binding fragment of any one of claims 1 to 24 for the preparation of a medicament for reducing autoimmunity or inflammation.

43. Use of the antibody or antigen-binding fragment of any one of claims 1 to 24 in treating an autoimmune or inflammatory disease associated with IL-7 and/or TSLP.

44. Use of the antibody or antigen-binding fragment of any one of claims 1 to 24 for the preparation of a medicament for treating an autoimmune or inflammatory disease associated with IL-7 and/or TSLP.

45. The use of claim 43 or 44, wherein the autoimmune or inflammatory disease is UC.


SEQUENCE LISTING (46 sequences)

SEQ ID NO: 1 - protein, 459 aa, Homo sapiens
  REGION 1..459: note = Human IL-7Ra
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 2 - protein, 459 aa, Macaca fascicularis
  REGION 1..459: note = Cynomolgus IL-7Ra
MTILGTTFGMVFSLLQVVSGESGYAQNGDLEDAELDDYSFSCYSQLEVNGSQHSLTCAFEDPDVNTTNLEFEICGALVEVKCLSFRKLQEIYFIETKKFLLIGKSNICVKVGGKSLTCKKIDLTTIVKPEAPFDLSVIYREGANDFVVTFNTSHLQKKYVKVLMHDVAYRQEKDENKWMHVNLSSTKLTLLQRNLQPEAMYEIKVRSIPDHYFKGFWSEWSPSYYFRTPEINNSPGEMDPILLTISLLSFFSVALLVILACVLWKKRIKPIVWPSLPDHKKTLEHLCKKPRKNLNVSFNPESFLDCQIHRVDDIQARDEVEGFLQDTFPQQLEESKKQRLGGDVQSPSCPSEDVVITPESFERDSSLRCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 3-7 - skipped (5 entries)

SEQ ID NO: 8 - protein, 15 aa, synthetic construct
  REGION 1..15: note = VL CDR1 Kabat, Chothia
RTSESVSIHLLHLMH

SEQ ID NO: 9 - protein, 7 aa, synthetic construct
  REGION 1..7: note = VL CDR2 Kabat, Chothia
GASNLES

SEQ ID NO: 10 - protein, 9 aa, synthetic construct
  REGION 1..9: note = VL CDR3 Kabat, Chothia
QQSIKDPYT

SEQ ID NO: 11 - protein, 7 aa, synthetic construct
  REGION 1..7: note = VH CDR1 Chothia
GYRFTDY

SEQ ID NO: 12 - protein, 5 aa, synthetic construct
  REGION 1..5: note = VH CDR1 Kabat
DYELH

SEQ ID NO: 13 - protein, 6 aa, synthetic construct
  REGION 1..6: note = VH CDR2 Chothia
DPKTGG

SEQ ID NO: 14 - protein, 17 aa, synthetic construct
  REGION 1..17: note = VH CDR2 Kabat
GIDPKTGGTAYNQKFKG

SEQ ID NO: 15 - protein, 9 aa, synthetic construct
  REGION 1..9: note = VH CDR3 Kabat, Chothia
SGGRWYFDV

SEQ ID NO: 16-17 - skipped (2 entries)

SEQ ID NO: 18 - protein, 111 aa, synthetic construct
  REGION 1..111: note = cmAb011 VL
DIVLTQSPASLAVSLGQRATISCRTSESVSIHLLHLMHWYQQKPGQPPKLLIYGASNLESGVPARFSGSGSETDFTLNIHPVEEEDAATYFCQQSIKDPYTFGGGTKLEIK

SEQ ID NO: 19 - protein, 118 aa, synthetic construct
  REGION 1..118: note = cmAb011 VH
QVQLQQSGAELMRPGASVTLSCKTSGYRFTDYELHWVKQTPVRGLEWIGGIDPKTGGTAYNQKFKGKAILTSDRSSSTTYMELRSLASEDSAVYYCTRSGGRWYFDVWGTGTTVTVSS

SEQ ID NO: 20 - protein, 111 aa, synthetic construct
  REGION 1..111: note = humanized VL1
DIVLTQSPASLAVSPGQRATITCRTSESVSIHLLHLMHWYQQKPGQPPKLLIYGASNLESGVPARFSGSGSGTDFTLTINPVEAEDTANYYCQQSIKDPYTFGGGTKVEIK

SEQ ID NO: 21 - protein, 111 aa, synthetic construct
  REGION 1..111: note = humanized VL2
DIVLTQSPASLAVSPGQRATITCRTSESVSIHLLHLMHWYQQKPGQPPKLLIYGASNLESGVPARFSGSGSETDFTLTINPVEAEDTANYYCQQSIKDPYTFGGGTKVEIK

SEQ ID NO: 22 - protein, 111 aa, synthetic construct
  REGION 1..111: note = humanized VL3
DIVMTQSPDSLAVSLGERATINCRTSESVSIHLLHLMHWYQQKPGQPPKLLIYGASNLESGVPDRFSGSGSGTDFTLTISSLQAEDVAVYYCQQSIKDPYTFGGGTKVEIK

SEQ ID NO: 23 - protein, 111 aa, synthetic construct
  REGION 1..111: note = humanized VL4
DIVLTQSPDSLAVSLGERATINCRTSESVSIHLLHLMHWYQQKPGQPPKLLIYGASNLESGVPDRFSGSGSETDFTLTISSLQAEDVAVYYCQQSIKDPYTFGGGTKVEIK

SEQ ID NO: 24-25 - skipped (2 entries)

SEQ ID NO: 26 - protein, 118 aa, synthetic construct
  REGION 1..118: note = humanized VH1
QVQLVQSGAEVKKPGASVKVSCKASGYRFTDYELHWVRQAPGQGLEWMGGIDPKTGGTAYNQKFKGRVTMTRDTSTSTVYMELSSLRSEDTAVYYCARSGGRWYFDVWGQGTTVTVSS

SEQ ID NO: 27 - protein, 118 aa, synthetic construct
  REGION 1..118: note = humanized VH2
QVQLVQSGAEVKKPGASVKVSCKASGYRFTDYELHWVRQAPGQGLEWMGGIDPKTGGTAYNQKFKGRVTMTSDRSTSTVYMELSSLRSEDTAVYYCARSGGRWYFDVWGQGTTVTVSS

SEQ ID NO: 28 - protein, 118 aa, synthetic construct
  REGION 1..118: note = humanized VH3
QVQLVQSGAEVKKPGASVKVSCKASGYRFTDYELHWVKQAPGQGLEWIGGIDPKTGGTAYNQKFKGRVTMTSDRSTSTVYMELSSLRSEDTAVYYCARSGGRWYFDVWGQGTTVTVSS

SEQ ID NO: 29 - protein, 118 aa, synthetic construct
  REGION 1..118: note = humanized VH4
QVQLVQSGAEVKKPGASVKVSCKASGYRFTDYELHWVRQAPGQGLEWMGGIDPKTGGTAYNQKFKGKVTLTSDRSTSTVYMELSSLRSEDTAVYYCARSGGRWYFDVWGQGTTVTVSS

SEQ ID NO: 30 - protein, 118 aa, synthetic construct
  REGION 1..118: note = humanized VH5
QVQLVQSGAEVKKPGASVKVSCKASGYRFTDYELHWVKQAPGQGLEWIGGIDPKTGGTAYNQKFKGKVTLTSDRSTSTVYMELSSLRSEDTAVYYCARSGGRWYFDVWGQGTTVTVSS

SEQ ID NO: 31-32 - skipped (2 entries)

SEQ ID NO: 33 - protein, 107 aa, Homo sapiens [sequence omitted]

SEQ ID NO: 34 - protein, 106 aa, Homo sapiens
  REGION 1..106: note = IgG CL Lambda
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 35 - protein, 329 aa, Homo sapiens
  REGION 1..329: note = IgG1 CH
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 325 aa, Homo sapiens
  REGION 1..325: note = IgG2 CH
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 37 - protein, 376 aa, Homo sapiens
  REGION 1..376: note = IgG3 CH
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 38 - protein, 326 aa, Homo sapiens
  REGION 1..326: note = IgG4 CH
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 39 - protein, 327 aa, synthetic construct
  REGION 1..327: note = human IgG4 CH S228P
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 5 aa, synthetic construct
  REGION 1..5: note = linker
  REPEAT 1..5: note = (GGGGS)n, n=1, 2, 3, 4, or 5
GGGGS

SEQ ID NO: 41 - protein, 5 aa, synthetic construct
  REGION 1..5: note = linker
  REPEAT 1..5: note = (EAAAK)n, n=1, 2, 3, 4, or 5
EAAAK

SEQ ID NO: 42 - protein, 5 aa, synthetic construct
  REGION 1..5: note = linker
  REPEAT 1..5: note = (PA)nPAP, n=0, 1, 2, 3, or 4
PAPAP

SEQ ID NO: 43 - protein, 214 aa, synthetic construct
  REGION 1..214: note = OSE127 light chain
DIQMTQSPSSLSASVGDRVTITCRTSEDIYQGLAWYQQKPGKAPKLLLYSANTLHIGVPSRFSGSGSGTDYTLTISSLQPEDFATYYCQQYYDYPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 449 aa, synthetic construct
  REGION 1..449: note = OSE127 heavy chain
QVQLVESGGGLVKPGGSLRLSCAVSGFTLSDYYMAWIRQAPGKGLEWVSTISASGLRTYYPDSVKGRFTISRDNAKNSLYLQMNSLRAEDTAVYYCARPLSAHYGFNYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 45 - protein, 216 aa, synthetic construct
  REGION 1..216: note = ADX-914 light chain
AIQLTQSPSSLSASVGDRVTITCRASQGISSALAWYQQKPGKAPKLLIYDASSLESGVPSRFSGSGSGTDFTLTISSLQPEDFATYYCQQFNSYPLWITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 450 aa, synthetic construct
  REGION 1..450: note = ADX-914 heavy chain
EVQLVESGGGLVQPGRSLRLSCAASGFTFDDHAMHWVRQAPGKGLEWVSGISWNSRGIGYADSVKGRFTIFRDNAKNSLYLQMNSLRAEDTALYYCAKDEYSRGYYVLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG